(19) United States
(12) Patent Application Publication
(10) Pub. No.: US 2024/0218010 A1
(43) Pub. Date: Jul. 4, 2024

(54) Methods of Producing Sulfated Oligosaccharide Derivatives and Intermediates Thereof

(71) Applicant: PROGEN PG500 SERIES PTY LTD, Darra (AU)

(72) Inventors: Paul Newton HANDLEY, Darra (AU); Tomislav KAROLI, Darra (AU); Jessica Anne ROWLEY, Darra (AU); Helen FRANKS, Reading Brookshire (GB); Alexander WEYMOUTH-WILSON, Reading Berkshire (GB); Robert CLARKSON, Reading Berkshire (GB); Laura WALLIS, Reading Berkshire (GB); Aileen WHITE, Reading Berkshire (GB); Andrew TYRRELL, Reading Berkshire (GB); Helen GALE, Reading Berkshire (GB); Phillip GREENWOOD, Reading Berkshire (GB)

(21) Appl. No.: 18/548,660

(22) PCT Filed: Mar. 4, 2022
(86) PCT No.: PCT/AU2022/050183

§ 371 Date: Sep. 1, 2023

(30) Foreign Application Priority Data
Mar. 4, 2021 (AU) 2021900614

(51) Int. Cl. C07J 31/00; C07J 17/00

(52) U.S. Cl. CPC C07J 31/006; C07J 17/005

(57) ABSTRACT

The invention relates inter alia to methods of producing sulfated oligosaccharide derivatives and intermediates thereof. The sulfated oligosaccharide derivatives may be represented by the following formula (I):

[X]ₙY−ZR¹R²Formula (I)

wherein:
X and Y are any D- or L-hexose or pentose, wherein each hydroxyl group not involved in a glycosidic linkage is substituted by a group W, and Y has an anomeric carbon atom;
W is SO₃M, and M is any pharmaceutically acceptable cation;
n is an integer from 2 to 6;
Z is O, and is linked to the anomeric carbon atom of Y; and
R¹R² together form a lipophilic moiety.

### CROSS REFERENCE

The present application claims priority to Australian provisional application no. 2021900614, filed 4 Mar. 2021, which is incorporated herein by reference in its entirety.

### FIELD

The invention relates inter alia to methods of producing sulfated oligosaccharide derivatives and intermediates thereof.

### BACKGROUND

It will be clearly understood that, if a prior art publication is referred to herein, this reference does not constitute an admission that the publication forms part of the common general knowledge in the art in Australia or in any other country.

WO2009049370A1 discloses the synthesis, characterisation and biological testing of a class of sulfated oligosaccharides having a hydrophobic aglycon. This class of modified sulfated oligosaccharides has been shown to have promising anti-cancer, anti-inflammatory and anti-viral activity. In particular, compounds within this class are able to inhibit the protein heparanase and growth factors, such as FGF-1, FGF-2, and VEGF, thereby possessing potent anti-angiogenic and anti-inflammatory properties. In addition, these compounds exhibit promising anti-viral activity against herpes simplex virus-1 (HSV-1), HSV-2, respiratory syncytial virus (RSV), human immunodeficiency virus (HIV), Ross River virus, Dengue virus and SARS-CoV-2.

For example, one compound within this class, Compound 1 (PG545/pixatimod), is undergoing Phase 1 clinical trials in oncology, both as a single treatment and in combination with nivolumab. This compound has been shown to exert potent immunomodulatory properties leading to the activation of innate immunity. Compound 1 is a fully sulfated maltotetraoside with a cholestanol aglycon.

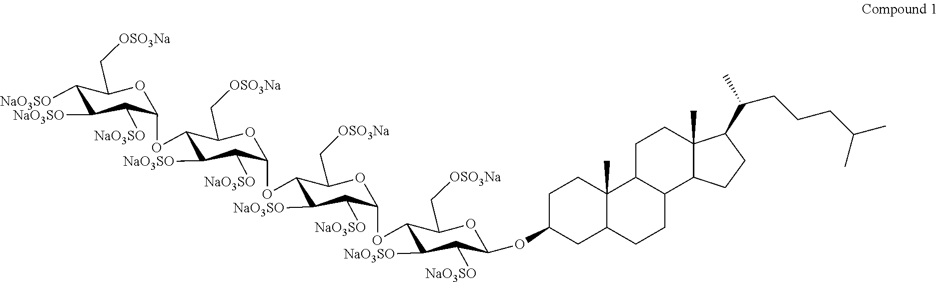

The synthesis of Compound 1 and other modified sulfated oligosaccharide compounds in the class includes a number of steps from their oligosaccharide starting materials, many of which require chromatographic separation steps which can be prohibitively expensive for synthesis on a commercial scale. In addition, many of the oligosaccharide starting materials are prohibitively expensive if purchased in a purified form, and are typically only available at a low cost as a mixture of oligosaccharides.

For example, the oligosaccharide starting material for the production of Compound 1 is maltotetraose, which is available in a pure form at a high cost, or at a low cost in a syrup comprising maltotetraose in >50% purity (w/w) on a dry weight basis. The syrup also includes various other oligosaccharide impurities (such as maltotriose and maltobiose) that react in a similar manner to maltotetraose, yielding impurities with similar properties to the intermediate and final products in the synthesis of Compound 1, and requiring expensive chromatographic separation steps which are not feasible for the commercial production of this compound.

### SUMMARY OF INVENTION

In various embodiments, the present invention is directed to a method for producing such sulfated oligosaccharide compounds, or to methods of producing intermediates for the production of such compounds, or to such intermediates, which may at least partially overcome at least one of the abovementioned disadvantages or provide the consumer with a useful or commercial choice. In one embodiment, the invention may at least partially provide new, more efficient methods for the synthesis of this class of modified sulfated oligosaccharide compounds, such as through using low cost starting materials and/or avoiding expensive chromatographic steps. In one embodiment, the invention may at least partially provide methods that are suitable for the synthesis of this class of modified sulfated oligosaccharide compounds on a commercial scale, optionally having a purity sufficient for administration to a mammal.

Disclosed herein are novel and efficient methods for the synthesis of sulfated oligosaccharide derivatives which may be suitable for use in production of such compounds on a commercial scale, optionally that avoid the need for chromatographic separation steps. Also disclosed herein are novel intermediate compounds formed in the synthesis of said sulfated oligosaccharide derivatives.

According to a first aspect there is provided a method of producing a compound of Formula (I),

[X]ₙY−ZR¹R² Formula (I)

wherein:
X and Y are any D- or L-hexose or pentose, wherein each hydroxyl group not involved in a glycosidic linkage is substituted by a group W, and Y has an anomeric carbon atom;
W is SO₃M, and M is any pharmaceutically acceptable cation;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH−R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
   the method comprising:
(A1) preparing a mixture comprising a compound of Formula (II), a reaction liquid, and a sulfur trioxide complex,

[X]ₙY—ZR¹R² Formula (II)

wherein:
n, Z, R¹ and R² are as defined in the compound of Formula (I), and
   X and Y are any D- or L-hexose or pentose, wherein each hydroxyl group not involved in a glycosidic linkage is substituted by a group W, Y has an anomeric carbon atom, and W is H;
(A2) separating a solid from the mixture; and
(A3) washing the solid with a dipolar aprotic wash solvent to produce the compound of Formula (I).

The following options may be used in conjunction with the first aspect, either individually or in any suitable combination.

In certain embodiments according to the first aspect, the reaction liquid is a dipolar aprotic solvent. The reaction liquid may comprise at least 70%, 80%, 90% or 95% dimethylformamide. The reaction liquid may be dimethylformamide. In certain embodiments of the first aspect, the reaction liquid comprises one or more solvents selected from the group consisting of DMF, HMPA, TPPA, DMI, DMPU, tetramethylurea, DMA, and NMP, or a combination thereof, preferably DMF.

In certain embodiments according to the first aspect, the reaction liquid and the dipolar aprotic wash solvent comprise the same solvent or solvents. Alternatively, in other embodiments, the reaction liquid and the dipolar aprotic wash solvent comprise a different solvent or solvents.

In certain embodiments according to the first aspect, the mass ratio of the reaction liquid to the sulfur trioxide complex in the mixture at step (A1) may be from about 1 to about 6, or from about 2 to about 5, about 2.5 to about 4.5, or about 3 to about 4. It may be, for example, about 1, 2, 2.5, 3, 3.1, 3.2, 3.3, 3.4, 3.5, 3.6, 3.7, 3.8, 3.9, 4, 4.5, 5, or 6.

In certain embodiments of the first aspect, the sulfur trioxide complex is a pyridine complex. In this case the sulfation reaction yields the compound of Formula (I) in its pyridinium salt form (i.e. where M is pyridinium).

In certain embodiments of the first aspect, the sulfur trioxide complex is used in excess. For example, from about 1.1 to about 6, or about 2 to about 5, about 2.5 to about 4, or about 1.1, 1.2, 1.5, 2, 2.5, 3, 4, 5, or 6 mole equivalents, preferably about 3 mole equivalents, of sulfur trioxide complex per hydroxyl group may be used in the sulfation reaction. The presence of excess sulfur trioxide complex in the reaction mixture may ensure complete sulfation of all hydroxyl groups, and further may influence the solubility of the compound of Formula (I) (in the reaction liquid) that is prepared by the sulfation reaction.

In certain embodiments according to the first aspect, the dipolar aprotic wash solvent comprises dimethylformamide. The dipolar aprotic wash solvent may comprise at least 70%, 80%, 90% or 95% dimethylformamide. The dipolar aprotic wash solvent may be dimethylformamide.

In certain embodiments of the first aspect, the sulfur trioxide complex is a pyridine, dioxane, trimethylamine, triethylamine, dimethylaniline, thioxane, 2-methylpyridine, quinoline, or DMF complex, or is a mixture thereof. In one embodiment, the sulfur trioxide complex comprises a pyridine complex. In a preferred embodiment, the sulfur trioxide complex is a pyridine complex.

In certain embodiments of the first aspect, the dipolar aprotic wash solvent comprises one or more solvents selected from the group consisting of DMF, HMPA, TPPA, DMI, DMPU, tetramethylurea, DMA, and NMP, or a combination thereof, preferably DMF.

The inventors of the present invention have surprisingly discovered that by precipitating and washing the crude reaction product comprising the compound of Formula (I) after step (A1) with a dipolar aprotic wash solvent, such as DMF, unwanted sulfated homologues comprising fewer monosaccharide units than the compound of Formula (I) (i.e. compounds having n or fewer monosaccharide units) may be removed from the crude reaction product. Additionally, or alternatively, the inventors have surprisingly found that unwanted incompletely sulfated products (i.e. where not all W are SO₃M, but instead some remain as H) may be removed from the crude reaction product using this precipitation and wash step. This may improve the purity of the compound of Formula (I) in the product or provide a purified product comprising the compound of Formula (I).

In certain embodiments of the first aspect, the method comprises one or more of the following steps to form the compound of Formula (I):
1. Mixing the compound of Formula (II) and a sulfur trioxide complex, optionally SO₃.Py, in a dipolar aprotic solvent (optionally selected from the group consisting of DMF, HMPA, TPPA, DMI, DMPU, tetramethylurea, DMA, and NMP, preferably DMF), optionally wherein the mass ratio of dipolar aprotic solvent to sulfur trioxide complex is from 1-6, 2-5, or 3-4; under an inert gas (optionally N₂), optionally from 5-40° C., 10-20° C., or 15-25° C. for 5-120 min, 10-60 min, or 25-40 min; then at 30-70° C., 35-60° C., or 40-45° C. for 4-48 h, 8-32 h, or 16-24 h;
2. Allowing a suspension to settle and removing the supernatant to form a first solid;
3. Slurrying the first solid with a dipolar aprotic solvent (optionally selected from the group consisting of DMF, HMPA, TPPA, DMI, DMPU, tetramethylurea, DMA, and NMP, preferably DMF), optionally with one or more modifiers (such as additional sulfur trioxide complex) and removing the supernatant to form a second solid;
4. Dissolving the second solid in water to form a solution, and adjusting the pH to 7-11, 7.5-10.5, or 8.0-10.0 using a base, optionally 10% w/w aq. NaOH, and maintaining the temperature below 40, 30, or 25° C.;
5. Adding an alcohol, optionally EtOH to the solution at 5-50° C., 10-40° C., 10-30° C., or 15-25° C. to form a suspension;
6. Filtering the suspension to form a filter cake, and washing the filter cake with an alcohol, optionally IPA;
7. Slurrying the filter cake with an alcohol, optionally IPA at 5-50° C., 10-40° C., 10-30° C., or 15-25° C. before filtering and washing with an alcohol (optionally IPA) to form a purified solid;
8. Dissolving the solid in an aqueous base (optionally NaHCO₃ in H₂O) to form a purified solution;
9. Performing membrane filtration (optionally ultrafiltration/diafiltration) on the purified solution (optionally using a 1 kDa, 1.5 kDa, or 2 kDa MWCO filter against a salt solution, optionally NaCl in H₂O, or more preferably an aqueous base, optionally NaHCO₃ in H₂O) to form a retentate;
10. Concentrating the retentate and adding it to an alcohol, optionally IPA to form a purified suspension; and
11. Filtering and drying the purified suspension to form the compound of Formula (I).

According to a second aspect there is provided a method of producing a compound of Formula (II),

[X]ₙY—ZR¹R² Formula (II)

wherein:
X and Y are any D- or L-hexose or pentose, wherein each hydroxyl group not involved in a glycosidic linkage is substituted by a group W, Y has an anomeric carbon atom, and W is H;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
   the method comprising:
(B1) mixing a compound of Formula (III) and a deprotecting agent to form the compound of Formula (II),

[X]ₙY−ZR¹R² Formula (III)
wherein:
   n, Z, R¹ and R² are as defined in the compound of Formula (II), and
   X and Y are any D- or L-hexose or pentose, wherein each hydroxyl group not involved in a glycosidic linkage is substituted by a group W, Y has an anomeric carbon atom, and W is a protecting group;
(B2) performing a membrane filtration to separate the compound of Formula (II) from one or more impurity;
   wherein the membrane filtration uses a membrane with a pore size which is at least twice the molecular weight of the compound of Formula (II).

The following options may be used in conjunction with the second aspect, either individually or in any suitable combination.

In certain embodiments of the second aspect, the membrane filtration uses a membrane with a pore size which is at least five times the molecular weight of the compound of Formula (II).

A person of skill in the art will understand that step (B1) is a deprotection step, in that the protecting group of the compound of Formula (III) is removed during the step. A person of skill in the art will understand that the protecting group may be any hydroxyl protecting group, such as those known in the art and, for example, described in Chapter 2 of Protecting Groups, by Philip J. Kocienski, Thieme, 2000, and further said protecting group may be removed by any conditions known in the art, such as using procedures described in the before referenced chapter.

In certain embodiments of the second aspect, step (B1) is performed using a base, preferably NaOMe in MeOH, or NaOH in MeOH.

In certain embodiments of the second aspect, the membrane filtration of step (B2) is an ultrafiltration.

In certain embodiments of the second aspect, the membrane filtration of step (B2) is a diafiltration, wherein water is added to dilute the retentate as it becomes more concentrated during the filtration process.

In certain embodiments of the second aspect, the membrane filtration of step (B2) uses a cellulose membrane or a polyethersulfone membrane, preferably a regenerated cellulose membrane.

In certain embodiments of the second aspect, the membrane filtration of step (B2) uses a membrane with a pore size or molecular weight cut off (MWCO) which is at least about two times the molecular weight of the compound of Formula (II), or at least about 5, 10, 15, 20, 25, or 30 times the molecular weight of the compound of Formula (II).

In certain embodiments of the second aspect, the membrane filtration of step (B2) uses a membrane with a pore size or molecular weight cut off (MWCO) of from about 2 kDa to about 50 kDa, about 5 kDa to about 40 kDa, about 10 kDa to about 40 kDa, about 20 kDa to about 30 kDa, or about 25 kDa to about 35 kDa. It may be, for example, about 2, 5, 10, 11, 12, 15, 20, 25, 26, 27, 28, 29, 30, 31, 32, 35, 40, or 50 kDa.

The inventors of the present invention have surprising found that the polyol compound of Formula (II) is strongly retained by a membrane having a pore size that is larger than the molecular weight of the compound of Formula (II), allowing low-molecular weight contaminants to pass through and be removed.

Without being bound by theory, the amphiphilic nature of the polyol may cause it to form large micellar structures in aqueous solution that enable it to be retained by the large pore-size membrane.

One benefit of ultrafiltration as a purification technique is that the purification is dependent on size, rather than on the chemical nature of each impurity. Typically, only molecules which form part of the micellar structure are retained by the membrane, and all other components below the molecular weight cut-off are removed, whether they be solvents, or inorganic or organic impurities. In particular, the inventors have surprisingly found that the membrane filtration can remove unreacted hemiacetal by-products (i.e. compounds of Formula II where R¹ is a bond, and R² is H) that would otherwise be difficult to separate from the desired product.

In certain embodiments of the second aspect, the method comprises one or more of the following steps to produce the compound of Formula (II):
1. Contacting a compound of Formula (III) in a polar aprotic solvent, optionally THF, and an alcohol, optionally MeOH, with 10, 20, 25, 30, 35, or 40% w/w of an alkoxide or hydroxide in an alcohol, optionally NaOMe in MeOH, or NaOH in MeOH, under an inert atmosphere, preferably under N₂, to form a reaction mixture;
2. Stirring said mixture at 30-80, 40-70, or 50-60° C. for 1-8, 2-7, 2-5, or 3-3.5 h, before cooling the mixture to 0-40, 5-30, 10-25, or 15-25° C. and stirring for a further 8-48, 12-32, or 16-24 h;
3. Adjusting the pH of the mixture to 4-9, 5-8, or 6-8 with an acid, preferably acetic acid such as glacial AcOH;
4. Concentrating the mixture, optionally under vacuum at 20-60, 30-50, 40-50, 40, 45, or 50° C.;
5. Adding the concentrated mixture to a polar aprotic solvent, optionally EtOAc at 0-40, 5-30, 10-25, or 15-25° C. to form a precipitate;
6. Filtering the precipitate to form a filter cake, and washing said filter cake with a polar aprotic solvent, optionally EtOAc;
7. Drying the filter cake to form a crude mixture;
8. Adding the crude mixture to water to form a solution;
9. Filtering the solution, optionally using an ultrafiltration/diafiltration with a 5, 10, 15, 20, 25, or 30 kDa MWCO membrane to form a product solution;
10. Concentrating the product solution and adding a polar aprotic solvent, optionally acetonitrile to precipitate a product;
11. Filtering and washing the product with a polar aprotic solvent, optionally acetonitrile/EtOAc, and then a polar aprotic solvent, optionally EtOAc, to form a solid;
12. Slurrying the solid in a polar aprotic solvent, optionally EtOAc, filtering and washing said solid with a polar aprotic solvent, optionally EtOAc; and
13. Drying the solid to form a compound of Formula (II).

In certain embodiments, the method of the first aspect can follow the method of the second aspect, and any one or more of the features of the method of the first aspect may therefore be applied to the method of the second aspect.

In certain embodiments, the method of the second aspect can precede the method of the first aspect, and any one or more of the features of the method of the second aspect may therefore be applied to the method of the first aspect.

According to a third aspect there is provided a method of producing a compound of Formula (III),

[X]ₙY−ZR¹R²Formula (III)

wherein:
X and Y are any D- or L-hexose or pentose, wherein each hydroxyl group not involved in a glycosidic linkage is substituted by a group W, Y has an anomeric carbon atom, and W is a protecting group;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—
R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
   the method comprising:
(C₁) reacting a compound of Formula (IV), with a compound of Formula (V) to form a compound of Formula (III);

[X]ₙY−R³ Formula (IV)

wherein:
X, Y and n are as defined in the compound of Formula (III), and
R³ is an optionally substituted thioaryl group, and is linked to the anomeric carbon atom of Y;

HOR¹R² Formula (V)

wherein:
R¹ and R² are as defined in the compound of Formula (III).

The following options may be used in conjunction with the third aspect, either individually or in any suitable combination.

Step (C₁) is a glycosylation reaction. A person of skill in the art will understand that a variety of glycosylation conditions may be employed for the glycosylation reaction, using a variety of reagents in a variety of solvents. A person of skill in the art will understand that the reagents, conditions and solvents will depend upon the structure of the protected oligosaccharide derivative, and in particular the specific leaving group used at R³ for the compound of Formula (IV), as well as the structure of the compound of Formula (V) to be glycosylated in the reaction. For example, if the leaving group is an optionally substituted thioaryl species, the glycosylation may be performed with an oxidizing agent, such as NIS, along with a Lewis acid, such as TMSOTf, in a halogenated solvent, such as dichloromethane, or in an ethereal solvent, such as TBME.

The inventors of the present invention have surprising found that by using optionally substituted thioaryl aglycon containing protected sugars (i.e. compounds of Formula (IV)), the glycosylation step (i.e. step (C₁)) has a number of advantages, such as one or more of the following:
(1) the compound of Formula (IV) may be formed in a single synthetic step from a suitable protected sugar (e.g. a perbenzoylated or peracetylated sugar), without having to selectively deprotect the anomeric hydroxyl group of the protected sugar;
(2) the anomeric selectivity of the glycosylation reaction may be high; and
(3) the stability of the compound of Formula (IV) may be higher compared with the stability of other leaving groups (such as trichloroacetimidate, and halogens).

In certain embodiments of the third aspect, step (C₁) comprises reacting the compound of Formula (V) with the glycosyl donor of Formula (IV) in the presence of NIS and TMSOTf. In certain embodiments, step (C₁) may be performed in the presence of a solvent, preferably a halogenated solvent, most preferably dichloromethane.

In certain embodiments of the third aspect, the glycosylation reaction is performed at a reduced temperature, such that the anomeric configuration of the product (being a compound of Formula (III)), can be controlled to maximise formation of the β-anomer. Accordingly, in certain embodiments of the third aspect, step (C₁) is performed at a temperature of below about 15° C., preferably below about 5° C. Alternatively, in cases where the α-anomer is preferred, step (C₁) may be performed at higher temperatures, for example, above about 15° C.

In certain embodiments of the third aspect, the method comprises one or more of the following steps to produce the compound of Formula (III):
1. Stirring a solution of the compound of Formula (IV) and the compound of Formula (V) in a halogenated solvent, optionally DCM under an inert gas, optionally N₂, for 30-60 min;
2. Cooling the reaction mixture to 0, 2.5, 5, 7.5, 10, 12.5, or 15° C.±5° C. and adding an oxidizing agent, optionally NIS, followed by a Lewis acid, optionally TMSOTf, maintaining the reaction temperature at <0, 5, 10, or 15° C. during addition;
3. Stirring the reaction mixture at 0, 2.5, 5, 7.5, or 10° C.±5° C. for 1, 1.5, 2, 2.5, or 3 h±15 min;
4. Adding a solution of an aqueous base and a reducing agent, optionally 5, 10, or 15% aq. KOH and 2, 5, or 10% aq. Na₂S₂O₃, to the reaction mixture;
5. Separating the aqueous phase, and washing the organic phase sequentially with an aqueous base and a reducing agent, optionally 5, 10, or 15% aq. KOH and 2, 5, or 10% aq. Na₂S₂O₃ solution, optionally followed by washing with an aqueous base, optionally 5% aq. NaHCO₃, and optionally followed by washing with an aqueous salt solution, optionally 5, 10, or 15% aq. NaCl;
6. Adding a polar aprotic solvent, optionally EtOAc to the organic phase to form a solution, and then concentrating the solution;
7. Adding the concentrated solution to a non-polar solvent, optionally heptane at 0-40, 5-35, 10-30, or 15-25° C. to form a precipitate; and
8. Filtering the precipitate, and washing it with a non-polar solvent, optionally heptane, before drying to form the compound of Formula (III).

In certain embodiments of the second or third aspect, the protecting group is an acetate or benzoate group.

In certain embodiments of the first, second or third aspect, R¹ is a bond, and R² is selected from the group consisting of optionally substituted steroidyl, optionally substituted C₁-C₁₀alkyl-NHCO—C₁-C₂₆ alkyl, and optionally substituted C₁-C₁₀ alkyl-CONH—C₁-C₂₆ alkyl; wherein said optional substituents are selected from the group consisting of C₁-C₁₂ alkyl, C₂-C₁₂ alkenyl, C₂-C₁₂ alkynyl, and halogen (or C₁-C₄ alkyl, C₂-C₄ alkenyl, C₂-C₄ alkynyl, and halogen).

In certain embodiments of the first, second or third aspect, R¹ is a bond, and R² is cholestanyl or propyl stearamide.

In certain embodiments of the third aspect, R³ is an optionally substituted thiotolyl. In another embodiment, R³ is thiotolyl. In a further embodiment, R³ is p-thiocresyl.

In certain embodiments, the method of the second and/or first aspect can follow the method of the third aspect, and any one or more of the features of the method of the second and/or first aspect may therefore be applied to the method of the third aspect.

In certain embodiments, the method of the third aspect can precede the method of the second and/or first aspect, and any one or more of the features of the method of the third aspect may therefore be applied to the method of the second and/or first aspect.

According to a fourth aspect there is provided a method of producing a compound of Formula (IV),

[X]ₙY−R³ Formula (IV)

wherein:
X and Y are any D- or L-hexose or pentose, wherein each hydroxyl group not involved in a glycosidic linkage is substituted by a group W, Y has an anomeric carbon atom, and W is a protecting group;
n is an integer from 2 to 6;
Z is O, and is linked to the anomeric carbon atom of Y; and
R³ is an optionally substituted thioaryl group, and is linked to the anomeric carbon atom of Y;
   the method comprising:
(D1) reacting a compound of Formula (VI), with an optionally substituted thioaryl compound to form a compound of Formula (IV);

[X]ₙY−V Formula(VI)

wherein:
X, Y and n are as defined in the compound of Formula (IV), and
V is W, and is linked to the anomeric carbon atom of Y.

The following options may be used in conjunction with the fourth aspect, either individually or in any suitable combination.

In certain embodiments of the fourth aspect, R³ is an optionally substituted thiotolyl.

In certain embodiments of the fourth aspect, R³ is thiotolyl.

In certain embodiments of the fourth aspect, R³ is p-thiocresyl.

In certain embodiments of the fourth aspect, step (D1) comprises reacting the compound of Formula (VI) with an optionally substituted thioaryl compound and a halogenated boron compound, such as BF₃. Optionally, the reactivity of the halogenated boron compound may be modulated by adding it in the form of a complex with another compound. For example, if a rapid reaction time is desired, the halogenated boron compound may be added as a complex with acetic acid. Alternatively, and more preferably, a less reactive ether complex, such as the diethyl etherate BF₃:OEt₂, may be used to slow down the reaction and minimise side-reactions. The mole ratio of optionally substituted thioaryl compound: halogenated boron compound may be from 1:1 to 1:1.6; or from 1:1 to 1:1.4; or from 1:1 to 1:1.3; or from 1:1.1 to 1:1.3; or about 1:1.2. The inventors have found that if too much halogenated boron compound is present in this step then this can be problematic.

In certain embodiments of the fourth aspect, step (D1) comprises reacting the compound of Formula (VI) with p-thiocresol and BF₃.

In certain embodiments of the fourth aspect, step (D1) is performed with a mole ratio of optionally substituted thioaryl compound: compound of Formula (IV) of from 1:1 to 1.2:1; or from 1.01:1 to 1.15:1; or from 1.01:1 to 1.1:1; or from 1.05:1 to 1.1:1; or about 1.09:1. The inventors have found that while a slight excess of thioaryl compound may be required to drive the reaction to completion, using greater relative amounts of thioaryl compound may be disadvantageous, since the thioaryl compound may need to be removed to prevent it from hindering the glycosylation reaction.

In certain embodiments of the fourth aspect, step (D1) is performed in a halogenated solvent, preferably dichloromethane. The method may comprise washing the solution, on completion of the reaction, with an alkali metal or alkaline earth hydroxide, such as NaOH or KOH. The inventors have found that a weaker base, such as NaHCO₃, may not be sufficient to completely remove the excess thioaryl compound by itself. The inventors have found that NaOH or KOH are sufficiently strong to deprotonate the thioaryl compound and allow it to be washed away, while reducing or preventing unwanted hydrolysis of the ester protecting groups (i.e. W in Formula IV)

In certain embodiments of the fourth aspect, the method further comprises a step of contacting the compound of Formula (IV) with an aqueous alcohol solution after step (D1), preferably a mixture of isopropanol and water, to precipitate said glycosyl donor in a solid form suitable for isolation.

In certain embodiments of the fourth aspect, the compound of Formula (VI) is prepared by reacting a mixture of oligosaccharides (especially maltooligosaccharides) (wherein said mixture may optionally comprise from 50% w/w to 95 w/w of a tetrasaccharide on a dry weight basis) with an acyl halide, acyl anhydride, aroyl halide or aroyl anhydride to thereby form a compound of Formula (VI).

In certain embodiments of the fourth aspect, the method comprises one or more of the following steps to form the compound of Formula (IV):
1. Dissolving the compound of Formula (VI) and thiocresol in dichloromethane and adjusting the temperature of the resultant solution to 35° C.;
2. Adding BF₃·OEt₂ while maintaining the reaction at <40° C. during the addition;
3. Stirring the mixture at 35° C.±5° C.;
4. Cooling the mixture to 20° C.±5° C. and quenching the mixture with Et₃N in EtOAc, maintaining the reaction temperature at <40° C. during the quenching;
5. Washing an organic phase of the mixture with the following: 10% aq. NaCl, then 10% aq. KOH/5% aq. Na₂S₂O₃, followed by 10% aq. NaCl;
6. Concentrating the organic phase by vacuum distillation;
7. Adding water and IPA to the reactor and adjusting the temperature to 15° C. 5° C.;
8. Adding the organic phase to the reactor to form a suspension;
9. Stirring the suspension at 15° C.±5° C. for 1 hour±30 minutes;
10. Removing the suspension from the reactor and filtering it to form a filter cake; washing the filter cake with IPA;
11. Adding heptane to the reactor and adjusting the temperature to 15° C.±5° C.; adding the filter cake to the reactor to form a suspension, and stirring the suspension for 30 minutes±15 minutes;
12. Removing the suspension from the reactor and filtering it prior to washing said suspension with heptane to form a solid; and
13. Drying the solid to thereby form the compound of Formula (IV).

In certain embodiments according to the first, second, third or fourth aspect, with respect to compounds of Formulas (I), (II), (III), (IV) and (VI), X and Y are monosaccharide units independently selected from the group consisting of glucose, mannose, altrose, allose, talose, galactose, idose, gulose, ribose, arabinose, xylose and lyxose, wherein each hydroxyl group of X and Y not involved in a glycosidic linkage is substituted by W.

In certain embodiments according to the first, second, third or fourth aspect, with respect to compounds of Formulas (I), (II), (III), (IV) and (VI), X and Y are glucose, wherein each hydroxyl group of X and Y not involved in a glycosidic linkage is substituted by W.

In certain embodiments according to the first, second, third or fourth aspect, with respect to compounds of Formulas (I), (II), (III), (IV) and (VI), n is 2 to 6, or from 2 to 4, or 2 or 3, or 3.

In certain embodiments according to the first, second, third or fourth aspect, with respect to compounds of Formulas (I), (II), (III), (IV) and (VI), each adjacent hexose or pentose in [X]ₙY is connected with a 1,4 glycosidic linkage.

In certain embodiments according to the first, second, third or fourth aspect, with respect to compounds of Formulas (I), (II), (III), (IV) and (VI), each adjacent hexose or pentose in [X]ₙY is connected with an α-1,4 glycosidic linkage.

In certain embodiments according to the first, second, third or fourth aspect, with respect to compounds of Formulas (I), (II), (III), (IV) or (VI), X and Y are glucose, wherein each hydroxyl group of X and Y not involved in a glycosidic linkage is substituted by W, and each adjacent glucose in [X]ₙY is connected with a 1,4 glycosidic linkage.

In certain embodiments according to the first, second, third or fourth aspect, with respect to compounds of Formulas (I), (II), (III), (IV) and (VI), [X]ₙY is selected from the group consisting of maltotriose, maltotetraose, maltopentaose, and maltohexaose, wherein each hydroxyl group of X and Y not involved in a glycosidic linkage is substituted by W.

In certain embodiments according to the first, second, third or fourth aspect, with respect to compounds of Formulas (I), (II), (III), (IV) and (VI), [X]ₙY is maltotetraose, wherein each hydroxyl group of X and Y not involved in a glycosidic linkage is substituted by W.

In certain embodiments of the first, second, third or fourth aspect, X and Y are glucose monosaccharide units.

In certain embodiments of the first, second, third or fourth aspect, X and Y are glucose monosaccharide units linked together with α-1,4 glycosidic linkages.

In certain embodiments of the first, second, third or fourth aspect, X and Y are mannose monosaccharide units.

In certain embodiments of the first, second, third or fourth aspect, n is 3 or 4.

In certain embodiments of the first, second, third or fourth aspect, n is 3.

In certain embodiments of the second, third or fourth aspect, with respect to compounds of Formulas (III), (IV), and (VI), the protecting group is an acyl or aroyl group.

In certain embodiments of the second, third or fourth aspect, with respect to compounds of Formulas (III), (IV), and (VI), the protecting group is an acetate or benzoate group.

In certain embodiments of the second, third or fourth aspect, with respect to compounds of Formulas (III), (IV), and (VI), the protecting group is a group selected from the table below, wherein X in the table indicates an oxygen atom from a sugar hydroxyl group (i.e. the protecting groups are attached as ester derivatives):

| Ester | Abbreviation | Structure |
|---|---|---|
| Acetyl | Ac | 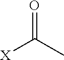 |
| Benzoyl | Bz | 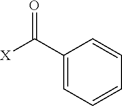 |
| p-nitrobenzoyl | p NO₂Bz | 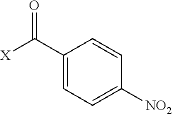 |
| p-bromobenzoyl | p BrBz | 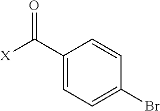 |
| p-methylbenzoyl | p MeBz | 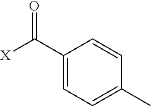 |
| biphenyl-4-carbonyl | p PhBz | 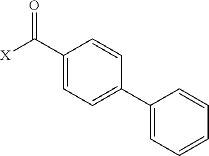 |
| 2-naphthoyl | NAP | 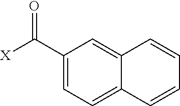 |

In certain embodiments, the method of the third, second and/or first aspect can follow the method of the fourth aspect, and any one or more of the features of the method of the third, second and/or first aspect may therefore be applied to the method of the fourth aspect.

In certain embodiments, the method of the fourth aspect can precede the method of the third, second and/or first aspect, and any one or more of the features of the method of the fourth aspect may therefore be applied to the method of the third, second and/or first aspect.

The overall synthetic scheme to produce compounds of Formula (I) from an oligosaccharide starting material according to the methods of one or more aspects of the present invention may be summarised according to the scheme depicted in FIG. 1.

A person of skill in the art will understand that the methods according to the fourth, third, second, and first aspects may be performed sequentially, optionally with one or more additional steps, in order to produce compounds of Formula (I) from a suitable oligosaccharide starting material.

In one embodiment, the method according to the first aspect, second, third, or fourth aspect may form a component of a method of the synthesis of a compound of Formula (I) from a suitable oligosaccharide starting material.

A suitable oligosaccharide starting material may comprise a natural or synthetic oligosaccharide. It may comprise, for example, one or more cellulose-derived oligosaccharides, such as cellohexaose, cellopentaose, cellotetraose, cellotriose, or a combination thereof. It may comprise, for example, one or more phosphomannan-derived oligosaccharides, such as phosphomannohexaose, phosphomannopentaose, phosphomannotetraose, phosphomannotriose, mannohexaose, mannopentaose, mannotetraose, mannotriose, or a combination thereof. It may comprise, for example, maltotriose, maltotetraose, maltopentaose, maltohexaose or a combination thereof. In certain embodiments it may comprise maltotetraose.

According to a fifth aspect, there is provided a method of producing a compound of Formula (VIII),

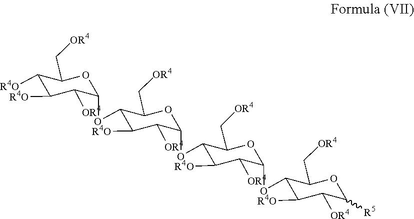

R⁴ is SO₃M, and M is any pharmaceutically acceptable cation;
R⁵ is OR¹R²;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—
R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
   the method comprising:

(E1) providing a starting material comprising a mixture of maltooligosaccharides, said starting material comprising from 50% w/w to 95% w/w of maltotetraose on a dry weight basis;

(E2) reacting the starting material with an acyl halide, acyl anhydride, aroyl halide, or aroyl anhydride to form a compound of Formula (VIII),

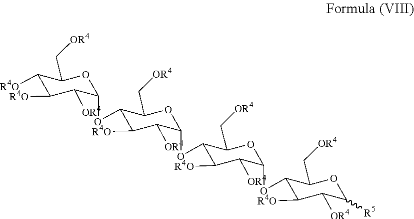

wherein:
R⁴ is an acyl or aroyl group, and
R⁵ is O-acyl or O-aroyl;

(E3) converting the compound of Formula (VIII) to a glycosyl donor of Formula (IX),

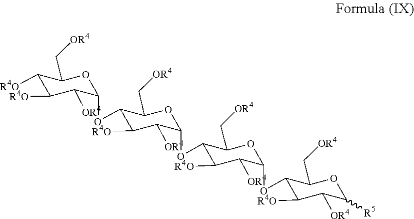

wherein:
R⁴ is as defined in the compound of Formula (VIII), and
R⁵ is a leaving group;

(E4) glycosylating a compound of Formula (X) with the glycosyl donor of Formula (IX) to form a compound of Formula (XI),

HOR¹R² Formula (X)

wherein R¹ and R² are as defined in the compound of Formula (VII),

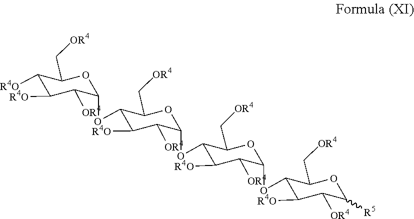

wherein:
R⁴ is as defined in the compound of Formula (VIII), and
R⁵ is as defined in the compound of Formula (VII);

(E5) removing acyl or aroyl protecting groups from the compound of Formula (XI) to form a first mixture comprising a compound of Formula (XII);

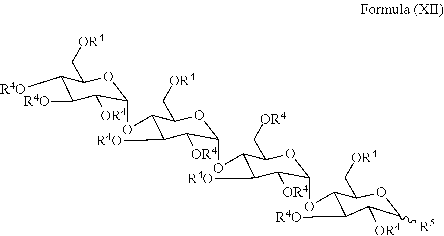

wherein:
R⁴ is OH;
R⁵ is as defined in the compound of Formula (VII);

(E6) subjecting the first mixture to membrane filtration to form a first purified composition comprising a compound of Formula (XII);

(E7) reacting the first purified composition with a sulfur trioxide complex in a reaction liquid to form a second mixture comprising a compound of Formula (VII); and

(E8) washing the second mixture with a dipolar aprotic wash solvent to form a second purified composition comprising a compound of Formula (VII).

The following options may be used in conjunction with the fifth aspect, either individually or in any suitable combination.

The inventors of the present invention have surprisingly found that by using the combination of steps set forth according to the fifth aspect, it is possible to produce a purified compound of Formula (VII) which is sufficiently pure for administration in a clinical trial setting, from a starting G4 syrup which comprises as low as only 50% maltotetraose (w/w on a dry weight basis) without the need for cost prohibitive chromatography steps. Accordingly, in certain embodiments of the fifth aspect, the method does not include a chromatography step.

In certain embodiments of the fifth aspect, the method is able to produce a compound of Formula (VII) having a purity of about 90% or more, about 95% or more, about 97% or more, about 98% or more, about 99% or more, about 99.5% or more, about 99.7% or more, or about 99.9% or more, from a starting G4 syrup having from about 50 to about 95% maltotetraose (w/w on a dry weight basis) without a chromatography step. In certain embodiments, the method is able to produce a compound of Formula (VII) having a purity of about 90% or more, about 95% or more, about 97% or more, or about 98% or more, from a G4 syrup having about 50% maltotetraose (w/w on a dry weight basis) without a chromatography step.

In certain embodiments of the fifth aspect, R¹ is a bond, and R² is selected from the group consisting of optionally substituted steroidyl, optionally substituted C₁-C₁₀ alkyl-NHCO—C₁-C₂₆ alkyl, and optionally substituted C₁-C₁₀ alkyl-CONH—C₁-C₂₆ alkyl; wherein said optional substituents are selected from the group consisting of C₁-C₁₂ alkyl, C₂-C₁₂ alkenyl, C₂-C₁₂ alkynyl, and halogen (or C₁-C₄ alkyl, C₂-C₄ alkenyl, C₂-C₄ alkynyl, and halogen).

In certain embodiments of the fifth aspect, R¹ is a bond, and R² is cholestanyl or propyl stearamide.

In certain embodiments of the fifth aspect, with respect to compounds of Formulas (VIII), (IX), and (XI), R⁴ is an acetate or benzoate group.

In certain embodiments of the fifth aspect, with respect to compounds of Formulas (VIII), (IX), and (XI), R⁴ is a group selected from the table below, wherein X in the table indicates an oxygen atom from a sugar hydroxyl group (i.e. the protecting groups are attached as ester derivatives):

| Ester | Abbreviation | Structure |
|---|---|---|
| Acetyl | Ac | 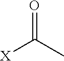 |
| Benzoyl | Bz | 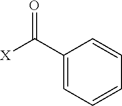 |
| p-nitrobenzoyl | p NO₂Bz | 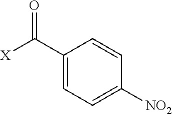 |
| p-bromobenzoyl | p BrBz | 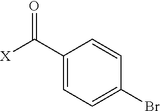 |
| p-methylbenzoyl | p MeBz | 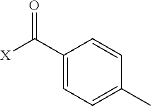 |
| biphenyl-4-carbonyl | p PhBz | 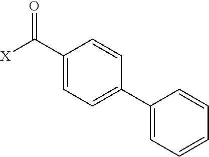 |
| 2-naphthoyl | NAP | 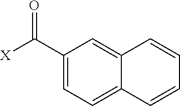 |

In certain embodiments of the fifth aspect, the starting material may comprise moisture (e.g. >5%, >10%, >15%, or >20% water), in which case the method may further comprise a step of drying the starting material prior to step (E2). The drying may be by any standard method known in the art, such as lyophilising, spray-drying, precipitation, e.g. acetone precipitation, or distillation, e.g. azeotropic distillation. In the case where the moisture is removed by azeotropic distillation, the azeotropic distillation may include distillation of a pyridine-water azeotrope. The starting material may be dried to <2%, <1.5%, or <1% moisture as determined by Karl Fischer titration prior to step (E2)

In certain embodiments of the fifth aspect, the starting material may comprise from about 50% w/w to about 90% w/w of maltotetraose on a dry weight basis, or it may comprise from about 50% w/w to about 90% w/w, about 50% w/w to about 80% w/w, about 50% w/w to about 70% w/w, about 50% w/w to about 60% w/w, about 60% w/w to about 90% w/w, about 70% w/w to about 90% w/w, or about 40% w/w to about 60% w/w of maltotetraose on a dry weight basis. It may comprise about 40, 45, 50, 55, 60, 65, 70, 80, or 90% w/w maltotetraose on a dry weight basis.

In certain embodiments of the fifth aspect, the maltotetraose starting material also contains one or more undesired oligosaccharides selected from the group consisting of maltotriose, maltose and glucose. The inventors of the present invention have surprisingly found that the combination of steps set forth according to the fifth aspect is particularly useful for removing impurities derived from these undesired oligosaccharides.

In certain embodiments of the fifth aspect, step (E2) is performed at a temperature of 100° C. or more.

In certain embodiments of the fifth aspect, the method further comprises a step of precipitating the compound of Formula (VIII). The precipitation may be performed by adding a strong solvent to the compound of Formula (VIII) after step (E2), followed by the addition of an anti-solvent. Suitable strong solvents include ethyl acetate, ethers especially THF, and aromatics such as toluene or pyridine. Suitable anti-solvents include water, alcohols, preferably isopropanol, and aliphatic hydrocarbons, such as heptane.

In certain embodiments of the fifth aspect, step (E2) comprises reacting the starting material with acetyl chloride, acetic anhydride, or benzoyl chloride, preferably benzoyl chloride.

In certain embodiments of the fifth aspect, step (E2) comprises reacting the starting material in the presence of a base, such as an acetate salt (e.g. sodium acetate) or pyridine; preferably pyridine.

In certain embodiments of the fifth aspect, the method comprises a step of adding an alcohol, preferably isopropanol, to the compound of Formula (VIII) to form a precipitate after step (E2).

In certain embodiments of the fifth aspect, the method comprises one or more of the following steps to form the compound of Formula (VIII):
1. Drying a solution of G4 syrup (comprising maltotetraose at ˜50, 60, 70, 80, 90, or 95% (w/w) by dry weight), optionally by azeotropic distillation in a polar aprotic solvent, optionally pyridine, to a moisture content of <2, 1.5, 1.3, 1, 0.5, or 0.1% (w/w) as determined by Karl Fischer (KF) titration;
2. Adding additional polar aprotic solvent, optionally pyridine, to the dried G4 syrup in a reaction vessel and heating the resultant solution to 60, 70, 80, 90, 100, or 110° C.;
3. Adding an aroyl chloride, optionally benzoyl chloride to the solution to form a reaction mixture, maintaining the reaction temperature at 80-130, 90-120, or 100-110° C.;
4. Stirring the reaction mixture, optionally under an inert gas, optionally nitrogen or argon, at 80-130, 90-120, 100-115, or 105-115° C.;
5. Cooling the reaction mixture to 50, 60, 70, 80, 90, or 100° C., then quenching the mixture by adding H₂O or an alcohol, optionally WA; maintaining the reaction mixture at <80, 90, 100, 110, or 120° C. during the quench;
6. Cooling the reaction mixture to 5, 10, 15, 20, 25, 30, 35, or 40° C. and adding a polar aprotic solvent, optionally ethyl acetate (EtOAc), to the reaction mixture;
7. Adding a salt solution, optionally brine (5, 10, or 15% aq. NaCl) to the reaction mixture to form a biphasic mixture, then separating a first aqueous phase from an organic phase;
8. Adding an acid, optionally 1, 2, 3 or 4M aq. HCl to the organic phase; then separating a second aqueous phase from the organic phase; repeating the acid, optionally 1, 2, 3 or 4M aq. HCl washes until the pH of the aqueous phase was <3, 2.5, 2, or 1.5;
9. Adding a salt solution, optionally brine (5, 10, or 15% aq. NaCl) to the organic phase and draining off the aqueous phase;
10. Concentrated the organic reaction mixture to approximately 4-5 volumes by distilling the polar aprotic solvent, optionally ethyl acetate (EtOAc), to form a crude solution;
11. Cooling the crude solution;
12. Adding an alcohol, optionally isopropyl alcohol (IPA), to a reactor and adjusting the temperature of the alcohol to 25, 20, 15, 10, or 5° C.;
13. Adding the crude solution to the reactor; and stirring the resulting suspension at 5, 10, 15, 20, or 25° C. for 10-120, 20-100, 25-80, or 30-60 minutes;
14. Filtering the suspension, optionally via vacuum filtration optionally through a filter cloth, to form a filter cake;
15. Washing the filter cake with an alcohol, optionally IPA, and then a non-polar solvent, optionally TBME-Heptane; and
16. Collecting and drying the resultant solid compound of Formula (VIII).

In certain embodiments of the fifth aspect, the leaving group is selected from the group consisting of OH, halide, optionally substituted acylimidate, optionally substituted arylimidate, optionally substituted C₁-C₈ heteroalkyl, optionally substituted C₂-C₈ heteroalkenyl, optionally substituted thioaryl, and optionally substituted heterocyclyl.

In certain embodiments of the fifth aspect, the leaving group is selected from the groups listed in the following table, wherein Glu indicates the reducing end anomeric carbon of a suitably protected maltooligosaccharide:

| Substituent | Abbreviation | Structure |
|---|---|---|
| Bromide | Br | Glu-Br |
| Fluoride | Fl | Glu-F |
| Hemiacetal | OH | Glu-OH |
| 1-Propanethiol | SPr | 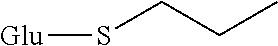 |
| 3-Chloro- benzenethiol | SPhCl | 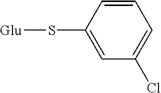 |
| p-Thiocresyl | Stol | 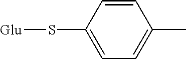 |
| Trichloroacetimidate | TCA | 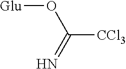 |
| N-Phenyltrifluoro- acetimidate | NPhTFA | 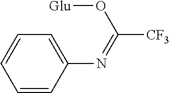 |
| 2-Mercapto- thiazoline | SThz | 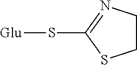 |

In certain embodiments of the fifth aspect, the leaving group is an optionally substituted thiotolyl.

In certain embodiments of the fifth aspect, the leaving group is thiotolyl.

In certain embodiments of the fifth aspect, the leaving group is p-thiocresyl.

In certain embodiments of the fifth aspect, step (E3) comprises reacting the compound of Formula (VIII) with an optionally substituted thioaryl compound (or arylthiol) and halogenated boron compound, such as BF₃ or a complex of BF₃ with another compound.

In certain embodiments of the fifth aspect, step (E3) comprises reacting the compound of Formula (VIII) with p-thiocresol and BF₃, optionally BF₃ diethyletherate.

In certain embodiments of the fifth aspect, step (E3) is performed in a halogenated solvent, preferably dichloromethane.

In certain embodiments of the fifth aspect, the method further comprises a step of contacting the glycosyl donor of Formula (IX) with an aqueous alcohol solution after step (E3), preferably a mixture of isopropanol and water, to precipitate said glycosyl donor.

In certain embodiments of the fifth aspect, step (E3) comprises one or more of the following steps to form the glycosyl donor:
1. Dissolving the compound of Formula (VIII) and an optionally substituted arylthiol, optionally thiocresol, in a halogenated solvent, optionally dichloromethane, and adjusting the temperature of the resultant solution to 20-50, 30-40, 30, 35, or 40° C.;
2. Adding a Lewis acid, optionally BF₃·OEt₂, while maintaining the reaction at <30, 35, 40, 45, or 50° C. during the addition;
3. Stirring the mixture at 20, 25, 30, 35, 45, or 50° C.±5° C.;
4. Cooling the mixture to 5, 10, 15, 20, or 25° C.±5° C. and quenching the mixture with a base, optionally Et₃N, in a polar aprotic solvent, optionally EtOAc, maintaining the reaction temperature at <20, 25, 30, 35, 40, 45, or 50° C. during the quenching;
5. Washing an organic phase of the mixture with the following: an aqueous salt solution, optionally 5, 10 or 15% aq. NaCl, then an aqueous basic solution comprising a reducing agent, optionally 5, 10, or 15% aq. KOH and 2.5, 5, 7.5 or 10% aq. Na₂S₂O₃, followed by an aqueous salt solution, optionally 5, 10 or 15% aq. NaCl;
6. Concentrating the organic phase, optionally by vacuum distillation;
7. Adding water and an alcohol, optionally IPA to the reactor and adjusting the temperature to 5, 10, 15, 20, 25, or 30° C. 5° C.;
8. Adding the organic phase to the reactor to form a suspension;
9. Stirring the suspension at 5, 10, 15, 20, 25, or 30° C.±5° C. for 0.5, 1, 1.5, 2, or 2.5 hour±30 minutes;
10. Removing the suspension from the reactor and filtering it to form a filter cake; washing the filter cake with an alcohol, optionally IPA;
11. Adding a non-polar solvent, optionally heptane, to the reactor and adjusting the temperature to 5, 10, 15, 20, or 25° C.±5° C.; adding the filter cake to the reactor to form a suspension, and stirring the suspension for 10, 20, 30, 40, 50, or 60 minutes 15 minutes;
12. Removing the suspension from the reactor and filtering it prior to washing said suspension with a non-polar solvent, optionally heptane to form a solid; and
13. Drying the solid to thereby form the glycosyl donor of Formula (IX).

Step (E4) is a glycosylation reaction. A person of skill in the art will understand that a variety of glycosylation conditions may be employed for the glycosylation reaction, using a variety of reagents in a variety of solvents. A person of skill in the art will understand that the reagents, conditions and solvents will depend upon the structure of the protected oligosaccharide derivative, and in particular the specific leaving group used at R⁵ for the compound of Formula (IX), as well as the structure of the compound of Formula (X) to be glycosylated in the reaction. For example, if the leaving group is a bromide, the glycosylation may be performed with ZnF₂ in toluene, acetonitrile, or TBME; if the leaving group is a fluoride, the glycosylation may be performed with TMSOTf in dichloromethane, acetonitrile, or an ethereal solvent such as diethyl ether; and if the leaving group is an optionally substituted thioaryl species, the glycosylation may be performed with NIS or another oxidizing agent, optionally also TMSOTf or another Lewis acidic promoter, in a halogenated solvent, such as dichloromethane, or an ethereal solvent such as THF or 2-methyl THF.

In certain embodiments of the fifth aspect, step (E4) comprises reacting the compound of Formula (X) with the glycosyl donor of Formula (IX) in the presence of NIS and TMSOTf. In certain embodiments, step (E4) may be performed in the presence of a solvent, preferably a halogenated solvent, most preferably dichloromethane.

In certain embodiments of the fifth aspect, the method further comprises a step of contacting the compound of Formula (XI) with an aliphatic solution after step (E4), preferably heptane, to precipitate said compound of Formula (XI).

In certain embodiments of the fifth aspect, the glycosylation reaction is performed at a reduced temperature, such that the anomeric configuration of the product (being a compound of Formula (XI)), can be controlled to maximise formation of the β-anomer. Accordingly, in certain embodiments of the fifth aspect, step (E4) is performed at a temperature of below about 15° C., preferably below about 5° C. Alternatively, in cases where the α-anomer is preferred, step (E4) may be performed at higher temperatures, for example, above about 15° C.

In certain embodiments of the fifth aspect, the method comprises one or more of the following steps to produce the compound of Formula (XI):
1. Stirring a solution of the compound of Formula (IX) and the compound of Formula (X) in a halogenated solvent, optionally DCM under N₂ for 30-60 min;
2. Cooling the reaction mixture to 0, 2.5, 5, 7.5, 10, 12.5, or 15° C.±5° C. and adding an oxidizing agent, optionally NIS, followed by a Lewis acid, optionally TMSOTf, maintaining the reaction temperature at <0, 5, 10, or 15° C. during addition;
3. Stirring the reaction mixture at 0, 2.5, 5, 7.5, or 10° C.±5° C. for 1, 1.5, 2, 2.5, or 3 h±15 min;
4. Adding a solution of an aqueous base and a reducing agent, optionally 5, 10, or 15% aq. KOH and 2, 5, or 10% aq. Na₂S₂O₃, to the reaction mixture;
5. Separating the aqueous phase, and washing the organic phase sequentially with an aqueous base and a reducing agent, optionally 5, 10, or 15% aq. KOH and 2, 5, or 10% aq. Na₂S₂O₃ solution, optionally followed by washing with an aqueous base, optionally 5% aq. NaHCO₃, and optionally followed by washing with an aqueous salt solution, optionally 5, 10, or 15% aq. NaCl;
6. Adding a polar aprotic solvent, optionally EtOAc to the organic phase to form a solution, and then concentrating the solution;
7. Adding the concentrated solution to a non-polar solvent, optionally heptane at 0-40, 5-35, 10-30, or 15-25° C. to form a precipitate; and
8. Filtering the precipitate, and washing it with a non-polar solvent, optionally heptane, before drying to form the compound of Formula (XI).

A person of skill in the art will understand that step (E5) is a deprotection step, in that the acyl or aroyl protecting groups of the compound of Formula (XI) are removed during the step. A person of skill in the art will understand that the protecting group may be removed using any conditions known in the art for the removal of such protecting groups, such as conditions, for example, described in Chapter 2 of Protecting Groups, by Philip J. Kocienski, Thieme, 2000.

In certain embodiments of the fifth aspect, step (E5) is performed using a base, preferably NaOMe in MeOH, or NaOH in MeOH.

In certain embodiments of the fifth aspect, the method further comprises a step of contacting the compound of Formula (XII) with a polar aprotic solvent after step (E5), preferably ethyl acetate, to precipitate said compound of Formula (XII).

In certain embodiments of the fifth aspect, the membrane filtration of step (E6) is an ultrafiltration/diafiltration.

In certain embodiments of the fifth aspect, the membrane filtration of step (E6) uses a cellulose membrane or a polyethersulfone membrane, preferably a regenerated cellulose membrane.

In certain embodiments of the fifth aspect, the membrane filtration of step (E6) uses a membrane with a pore size or molecular weight cut off (MWCO) which is at least about two times the molecular weight of the compound of Formula (XII), or at least about 5, 10, 15, 20, 25, or 30 times the molecular weight of the compound of Formula (XII).

In certain embodiments of the fifth aspect, the membrane filtration of step (E6) uses a membrane with a pore size or molecular weight cut off (MWCO) of from about 2 kDa to about 50 kDa, about 5 kDa to about 40 kDa, about 10 kDa to about 40 kDa, about 20 kDa to about 30 kDa, or about 25 kDa to about 35 kDa. It may be, for example, about 2, 5, 10, 11, 12, 15, 20, 25, 26, 27, 28, 29, 30, 31, 32, 35, 40, or 50 kDa.

The inventors of the present invention have surprising found that the polyol compound of Formula (XII) is strongly retained by a membrane having a pore size that is larger than the molecular weight of the compound of Formula (XII), allowing low-molecular weight contaminants to pass through and be removed.

Without being bound by theory, the amphiphilic nature of the polyol may cause it to form large micellular structures in aqueous solution that enable it to be retained by the larger pore size membrane.

One benefit of ultrafiltration as a purification technique is that the purification is dependent on size, rather than on the chemical nature of each impurity. Typically, only molecules which form part of the micellar structure are retained by the membrane, and all other components below the molecular weight cut-off are removed, whether they be solvents, or inorganic or organic impurities. In particular, the inventors have surprisingly found that the membrane filtration can remove unreacted hemiacetal by-products that would otherwise be difficult to separate from the desired product.

In certain embodiments of the fifth aspect, the method comprises one or more of the following steps to produce the compound of Formula (XII):
1. Contacting a compound of Formula (XI) in a polar aprotic solvent, optionally THF, and an alcohol, optionally MeOH, with 10, 20, 25, 30, 35, or 40% w/w of an alkoxide or hydroxide in an alcohol, optionally NaOMe in MeOH, or NaOH in MeOH, under an inert atmosphere, preferably under N₂, to form a reaction mixture;
2. Stirring said mixture at 30-80, 40-70, or 50-60° C. for 1-8, 2-7, 2-5, or 3-3.5 h, before cooling the mixture to 0-40, 5-30, 10-25, or 15-25° C. and stirring for a further 8-48, 12-32, or 16-24 h;
3. Adjusting the pH of the mixture to 4-9, 5-8, or 6-8 with an acid, preferably glacial AcOH;
4. Concentrating the mixture, optionally under vacuum at 20-60, 30-50, 40-50, 40, 45, or 50° C.;
5. Adding the concentrated mixture to a polar aprotic solvent, optionally EtOAc at 0-40, 5-30, 10-25, or 15-25° C. to form a precipitate;
6. Filtering the precipitate to form a filter cake, and washing said filter cake with a polar aprotic solvent, optionally EtOAc;
7. Drying the filter cake to form a crude mixture;
8. Adding the crude mixture to water to form a solution;
9. Filtering the solution, optionally using an ultrafiltration/diafiltration with a 5, 10, 15, 20, 25, or 30 kDa MWCO membrane to form a product solution;
10. Concentrating the product solution and adding a polar aprotic solvent, optionally acetonitrile to precipitate a product;
11. Filtering and washing the product with a polar aprotic solvent, optionally acetonitrile/EtOAc, and then a polar aprotic solvent, optionally EtOAc, to form a solid;
12. Slurrying the solid in a polar aprotic solvent, optionally EtOAc, filtering and washing said solid with a polar aprotic solvent, optionally EtOAc; and
13. Drying the solid to form a compound of Formula (XII).

The sulfur trioxide complex may act as a sulfating agent, which is capable of sulfating the sugar hydroxyl groups. A person of skill in the art will understand that the choice of the specific sulfur trioxide complex may be determined by the effect of the complexing agent on the solubility properties of the compound of Formula (VII) that is prepared by the sulfation reaction. In particular, some complexing agents may give rise to solubility properties that can be exploited so as to enable improvement in the ease of purification of the sulfated product. For example, a sulfur trioxide complex, such as sulfur trioxide pyridine, may be selected so as to form a salt form of a compound of Formula (VII), such as a pyridinium salt form, having reduced solubility in the reaction liquid as compared with the starting materials of the reaction, such that the compound of Formula (VII) forms a precipitate in the reaction mixture after its formation, or upon cooling of the reaction mixture.

In certain embodiments of the fifth aspect, the sulfur trioxide complex is a pyridine complex. In this case the sulfation reaction yields the compound of Formula (VII) in its pyridinium salt form (i.e. where M is pyridinium).

In certain embodiments of the fifth aspect, the sulfur trioxide complex is used in excess. For example, from about 1.1 to about 6, or about 2 to about 5, about 2.5 to about 4, or about 1.1, 1.2, 1.5, 2, 2.5, 3, 4, 5, or 6 mole equivalents, preferably about 3 mole equivalents, of sulfur trioxide complex per hydroxyl group may be used in the sulfation reaction. The presence of excess sulfur trioxide complex in the reaction mixture may ensure complete sulfation of all hydroxyl groups, and further may influence the solubility of the compound of Formula (VII) (in the reaction liquid) that is prepared by the sulfation reaction.

In certain embodiments according to the fifth aspect, the reaction liquid is a dipolar aprotic solvent. The reaction liquid may comprise at least 70%, 80%, 90% or 95% dimethylformamide. The reaction liquid may be dimethylformamide. In certain embodiments of the fifth aspect, the reaction liquid comprises one or more solvents selected from the group consisting of DMF, HMPA, TPPA, DMI, DMPU, tetramethylurea, DMA, and NMP, or a combination thereof, preferably DMF.

In certain embodiments according to the fifth aspect, the reaction liquid and the dipolar aprotic wash solvent comprise the same solvent or solvents. Alternatively, in other embodiments, the reaction liquid and the dipolar aprotic wash solvent comprise a different solvent or solvents.

In certain embodiments according to the fifth aspect, the mass ratio of the reaction liquid to the sulfur trioxide complex in the second mixture at step (E7) may be from about 1 to about 6, or from about 2 to about 5, about 2.5 to about 4.5, or about 3 to about 4. It may be, for example, about 1, 2, 2.5, 3, 3.1, 3.2, 3.3, 3.4, 3.5, 3.6, 3.7, 3.8, 3.9, 4, 4.5, 5, or 6.

In certain embodiments of the fifth aspect, the dipolar aprotic wash solvent comprises one or more solvents selected from the group consisting of DMF, HMPA, TPPA, DMI, DMPU, tetramethylurea, DMA, and NMP or a combination thereof, preferably DMF.

In certain embodiments, of the fifth aspect, the quantity of reaction liquid used is insufficient for complete solubility of the compound of Formula (VII) that is prepared by sulfation, resulting in precipitation of the crude product during the course of the sulfation reaction. For example, in the case where the reaction liquid is DMF, a mass of DMF that is about 2, 2.5, 3, 3.5, or 4 times the mass of the sulfur trioxide pyridine complex used may be used. The mass of the reaction liquid may depend on the solubility properties of the compound of Formula (VII) that is prepared by the sulfation reaction. For example, an amount of reaction liquid which is insufficient to completely solubilise the compound of Formula (VII) as it forms may be used in the reaction, such that the product (i.e. compound of Formula (VII)) forms a precipitate.

The inventors of the present invention have surprising discovered that by precipitating and washing the crude reaction product comprising the compound of Formula (VII), optionally in its pyridinium salt form, after step (E7) with a dipolar aprotic wash solvent, unwanted sulfated by-products comprising fewer monosaccharide units than the compound of Formula (VII) (i.e. having one, two or three monosaccharide units) may be removed from the crude reaction product, thereby providing a purified product comprising the compound of Formula (VII).

In certain embodiments of the fifth aspect, the method further comprises a step of converting the purified product comprising the compound of Formula (VII) to a different salt form, for example a pharmaceutically acceptable salt form.

In certain embodiments of the fifth aspect, the method further comprises a step of converting the purified product comprising the compound of Formula (VII) in its pyridinium salt form to the compound of Formula (VII) in its sodium salt form, by basification with aqueous sodium hydroxide followed by re-precipitation in a suitable solid form. In certain embodiments, the re-precipitation of purified product comprising the compound of Formula (VII) in its sodium salt form is performed by adding an alcohol, optionally ethanol, optionally also adding an additional sodium salt, optionally NaCl.

The inventors of the present invention have determined that successful re-precipitation of the purified compound of Formula (VII) may depend on balancing the quantity of residual dipolar aprotic solvent remaining from the wash step, against the quantity of water, alcohol and salt added, to give a finely divided solid suitable for isolation, for example by vacuum filtration.

In certain embodiments of the fifth aspect, the method comprises one or more of the following steps to form the compound of Formula (VII):
1. Mixing the compound of Formula (XII) and a sulfur trioxide complex, optionally SO₃.Py (optionally in a quantity of around 3 mole equivalents per hydroxyl group), in a dipolar aprotic solvent, optionally selected from DMF, HIVIPA, TPPA, DMI, DMPU, tetramethylurea, DMA, and NMP or a mixture thereof, preferably DMF (optionally in a quantity of around 3.5 times the mass of SO₃.Py added), under an inert gas, optionally N₂, optionally from 5-40° C., 10-20° C., or 15-25° C. for 5-120 min, 10-60 min, or 25-40 min; then at 30-70° C., 35-60° C., or 40-45° C. for 4-48 h, 8-32 h, or 16-24 h;
2. Allowing a suspension to settle and removing the supernatant to form a first solid;
3. Slurrying the first solid with a dipolar aprotic solvent, optionally selected from DMF, HMPA, TPPA, DMI, DMPU, tetramethylurea, DMA, and NMP or a mixture thereof, preferably DMF and removing or at least partially removing the supernatant to form a second solid; optionally leaving a controlled amount of supernatant to remain with the second solid, optionally around 5 weights;
4. Dissolving the second solid in water to form a solution, and adjusting the pH to 7-11, 7.5-10.5, or 8.0-10.0 using a base, optionally 10% w/w aq. NaOH, and maintaining the temperature below 40, 30, or 25° C.;
5. Adding an alcohol, optionally EtOH to the solution, and optionally adding a salt, optionally solid NaCl to the solution, at 5-50° C., 10-40° C., 10-30° C., or 15-25° C. to form a suspension;
6. Filtering the suspension to form a filter cake, and washing the filter cake with an alcohol, optionally IPA;
7. Slurrying the filter cake with an alcohol, optionally IPA at 5-50° C., 10-40° C., 10-30° C., or 15-25° C. before filtering and washing with an alcohol, optionally IPA to form a purified solid;
8. Dissolving the solid in water or an aqueous base, optionally NaHCO₃ in H₂O to form a purified solution;
9. Performing membrane filtration, optionally ultrafiltration/diafiltration on the purified solution, optionally using a 1 kDa, 1.5 kDa, or 2 kDa MWCO filter optionally against a salt solution, optionally NaCl in H₂O, and optionally an aqueous base, optionally NaHCO₃ in H₂O to form a retentate;
10. Concentrating the retentate and adding it to an alcohol, optionally IPA to form a purified suspension; and
11. Filtering and drying the purified suspension to form the compound of Formula (VII).

According to a sixth aspect, there is provided a compound of Formula (VII) produced according to the method of the fifth aspect.

According to a seventh aspect, there is provided a compound of Formula (I) produced according to the method of the first aspect.

According to an eighth aspect, there is provided a compound of Formula (XIII), or a salt thereof

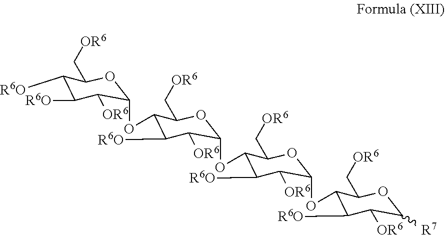

wherein:
R⁶ is an acyl or aroyl group; and
R⁷ is an optionally substituted thioaryl group.

The following options may be used in conjunction with the eighth aspect, either individually or in any suitable combination.

In a specific embodiment of the eighth aspect, R⁶ is benzoyl, and R⁷ is thiotolyl.

According to a ninth aspect there is provided use of the compound or salt thereof of the eighth aspect in the manufacture of a compound of Formula (VII);

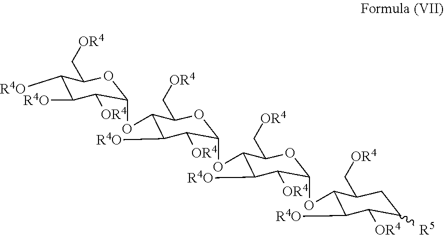

wherein:
R⁴ is SO₃M, and M is any pharmaceutically acceptable cation;
R⁵ is OR¹R²;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—
R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen; R¹⁰ is selected from the group consisting of C₁-C₆ alkyl, C₂-C₆ alkenyl1, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond.

The following options may be used in conjunction with the ninth aspect, either individually or in any suitable combination.

In certain embodiments of the first, fifth or ninth aspect, with respect to the compounds of Formulas (I) and (VII), M is potassium, ammonium, pyridinium, or sodium, preferably sodium.

In certain embodiments of the first, second, third, fifth or ninth aspect, with respect to the compounds of Formulas (I), (II), (III), (V), (VII), (XI), (X) and (XII), R¹ is C₁-C₆ alkyl, C₂-C₆ alkenyl, C₁-C₆ heteroalkyl, C₂-C₆ heteroalkenyl, or is a bond.

In certain embodiments of the first, second, third, fifth or ninth aspect, with respect to the compounds of Formulas (I), (II), (III), (V), (VII), (XI), (X) and (XII), R¹ is a bond.

In certain embodiments of the first, second, third, fifth or ninth aspect, with respect to the compounds of Formulas (I), (II), (III), (V), (VII), (XI), (X) and (XII), R² is selected from the group consisting of steroidyl optionally substituted by C₁-C₁₂ alkyl, C₁-C₃₆ alkyl, C₂-C₃₆ alkenyl, C₄-C₃₆ cycloalkyl, aryl, C₄-C₃₆ cycloalkenyl, R⁸—CONH—R⁹, R⁸—NHCO—R⁹, R⁸—CSNH—R⁹, R⁸—NHCS—R⁹, R⁸—CO—R⁹, C₁-C₃₆ heteroalkyl, C₂-C₃₆ heteroalkenyl, C₂-C₃₆ heteroalkynyl, and heteroaryl.

In certain embodiments of the first, second, third, fifth or ninth aspect, with respect to the compounds of Formulas (I), (II), (III), (V), (VII), (XI), (X) and (XII), R² is selected from the group consisting of steroidyl optionally substituted by C₁-C₁₂ alkyl, and R⁸—CONH—R⁹, R⁸—NHCO—R⁹, R⁸—CSNH—R⁹, R⁸—NHCS—R⁹, R⁸—CO—R⁹, C₁-C₃₆ heteroalkyl, C₂-C₃₆ heteroalkenyl, C₂-C₃₆ heteroalkynyl, and heteroaryl.

In certain embodiments of the first, second, third, fifth or ninth aspect, with respect to the compounds of Formulas (I), (II), (III), (V), (VII), (XI), (X) and (XII), R² is selected from the group consisting of optionally substituted steroidyl, optionally substituted C₁-C₁₀ alkyl-NHCO—C₁-C₂₆ alkyl, and optionally substituted C₁-C₁₀ alkyl-CONH—C₁-C₂₆ alkyl; wherein said optional substituents are selected from the group consisting of C₁-C₁₂ alkyl, C₂-C₁₂ alkenyl, C₂-C₁₂ alkynyl, and halogen.

In certain embodiments of the first, second, third, fifth or ninth aspect, with respect to the compounds of Formulas (I), (II), (III), (V), (VII), (XI), (X) and (XII), R¹ is a bond, and R² is selected from the group consisting of optionally substituted steroidyl, optionally substituted C₁-C₁₀ alkyl-NHCO—C₁-C₂₆ alkyl, and optionally substituted C₁-C₁₀ alkyl-CONH—C₁-C₂₆ alkyl; wherein said optional substituents are selected from the group consisting of C₁₂-C₄ alkyl, C₂-C₁₂ alkenyl, C₂-C₁₂ alkynyl, and halogen.

In certain embodiments of the first, second, third, fifth or ninth aspect, with respect to the compounds of Formulas (I), (II), (III), (V), (VII), (XI), (X) and (XII), R¹ is a bond, and R² comprises four fused carbocyclic rings, C₁-C₁₀ alkyl-CONH—C₁-C₂₆ alkyl, or C₁-C₁₀ alkyl-NHCO—C₁-C₂₆ alkyl.

In certain embodiments of the first, second, third, fifth or ninth aspect, with respect to the compounds of Formulas (I), (II), (III), (V), (VII), (XI), (X) and (XII), R¹ is a bond, and R² is selected from the group consisting of cholestanyl, and propylstearamide.

In certain embodiments of the first, second, third, fifth or ninth aspect, with respect to the compounds of Formulas (I), (II), (III), (V), (VII), (XI), (X) and (XII), R⁸ is selected from the group consisting of C₁-C₃₆ alkyl, C₂-C₃₆ alkenyl, C₁-C₃₆ heteroalkyl, and C₂-C₃₆ heteroalkenyl.

In certain embodiments of the first, second, third, fifth or ninth aspect, with respect to the compounds of Formulas (I), (II), (III), (V), (VII), (XI), (X) and (XII), R⁸ is C₁-C₃₆ alkyl.

In certain embodiments of the first, second, third, fifth or ninth aspect, with respect to the compounds of Formulas (I), (II), (III), (V), (VII), (XI), (X) and (XII), R⁸ is C₂-C₆ alkyl.

In certain embodiments of the first, second, third, fifth or ninth aspect, with respect to the compounds of Formulas (I), (II), (III), (V), (VII), (XI), (X) and (XII), R⁹ is selected from the group consisting of C₁-C₃₆ alkyl, C₂-C₃₆ alkenyl, C₁-C₃₆ heteroalkyl, and C₂-C₃₆ heteroalkenyl.

In certain embodiments of the first, second, third, fifth or ninth aspect, with respect to the compounds of Formulas (I), (II), (III), (V), (VII), (XI), (X) and (XII), R⁹ is C₁₀-C₂₆ alkyl.

In certain embodiments of the first, second, third, fifth or ninth aspect, with respect to the compounds of Formulas (I), (II), (III), (V), (VII), (XI), (X) and (XII), R⁹ is C₁₄-C₂₂ alkyl.

Any of the features described herein can be combined in any combination with any one or more of the other features described herein within the scope of the invention.

According to a tenth aspect, there is provided a compound of Formula (XIV), or a salt thereof

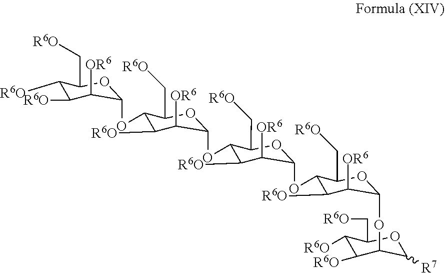

wherein:
R⁶ is an acyl or aroyl group; and
R⁷ is an optionally substituted thioaryl group.

The following options may be used in conjunction with the tenth aspect, either individually or in any suitable combination.

In a specific embodiment of the tenth aspect, R⁶ is acetyl, and R⁷ is thiotolyl.

Any of the features described herein can be combined in any combination with any one or more of the other features described herein within the scope of the invention.

### BRIEF DESCRIPTION OF DRAWINGS

FIG. 1: An example overall synthetic scheme to produce a compound of Formula (I).

FIG. 2: Representative micrograph of an example perbenzoylated intermediate compound in the synthesis of Compound 1. The compound is in the form of glassy amorphous beads, which is highly suitable for isolation by vacuum filtration.

FIG. 3: NMR spectra of M5AcSTo1: (A)¹H NMR spectrum; (B) and (C): HSQC NMR spectrum.

FIG. 4: NMR spectra of M5AcChol: (A)¹H NMR spectrum; (B), (C) and (D): HSQC NMR spectrum.

FIG. 5: NMR spectra of M5OHChol: (A)¹H NMR spectrum; (B) COSY NMR spectrum, and (C): HSQC NMR spectrum.

FIG. 6: NMR spectra of Compound 5: (A)¹H NMR spectrum; (B) COSY NMR spectrum, and (C): HSQC NMR spectrum.

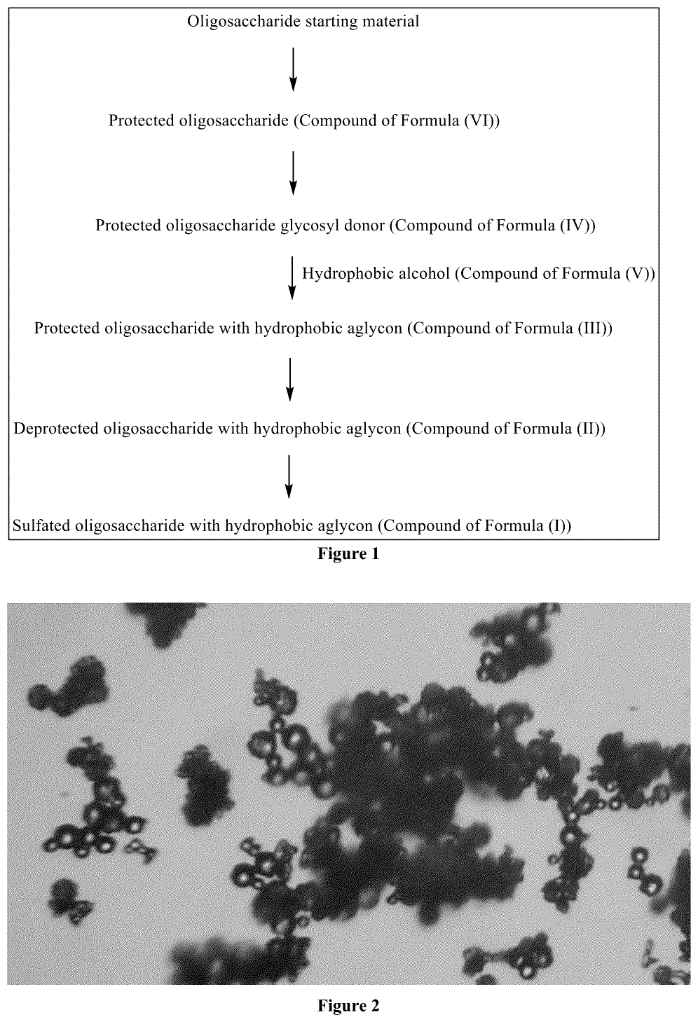

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### Abbreviations

AcOH—acetic acid; BzCl—benzoyl chloride; CE—capillary electrophoresis; DCM—dichloromethane; DMA— N,N-dimethylacetamide; DMAP— N,N-dimethyl aminopyridine; DMF N,N-dimethylformamide; DMI—1,3-dimethyl-2-imidazolidinone; DMSO—dimethyl sulfoxide; DMPU—N,N′-dimethylpropyleneurea; EtOAc—ethyl acetate; EtOH—ethanol; HMPA-hexamethyl phosphoramide; HSQC—Heteronuclear Single Quantum Coherence; IMS—industrial methylated spirits (ethanol with about 5% methanol); MeCN—acetonitrile; MeOH—methanol; MWCO—molecular weight cut-off, NaOMe—sodium methoxide; NIS— N-iodosuccinimide; NMP—N-methyl-2-pyrrolidone; py—pyridine; SFCA—surfactant-free cellulose acetate; STP—sulfur trioxide pyridine complex; TCA—trichloroacetimidate; THF—tetrahydrofuran; TBME—methyl tertiary butyl ether; TMSOTf—TMS triflate, or trimethylsilyl trifluoromethanesulfonate; TPPA—tripyrrolidinophosphoric acid; UF—ultrafiltration.

### Definitions

As used herein, the term “about”, is relative to the actual value stated, as will be appreciated by those of skill in the art, and allows for approximations, inaccuracies and limits of measurement under the relevant circumstances. Depending on context, it may allow a variation from the stated value of ±10%, +5%, +2%, +1%, +0.5%, +0.2%, +0.1%, +0.05%, +0.02%, or 0.01%.

As used herein, the term “comprising” indicates the presence of the specified integer(s), but allows for the possibility of other integers, unspecified. This term does not imply any particular proportion of the specified integers. Variations of the word “comprising”, such as “comprise” and “comprises”, have correspondingly similar meanings.

Unless defined otherwise, all technical and scientific terms used herein have the same meaning as would be commonly understood by those of ordinary skill in the art to which this invention belongs.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment.

Furthermore, the particular features, structures, or characteristics may be combined in any suitable manner in one or more combinations.

As used herein, the term “aroyl”, means a group selected from the group consisting of CO-aryl, and CO-heteroaryl, each of which may be optionally substituted with one or more groups independently selected from C₁₋₆ alkyl, C₁₋₆ alkenyl, nitro, cyano, NHR14, N(R¹⁴)₂, NHCOR¹⁴, CF₃, aryl, heteroaryl, and halogen; wherein R¹⁴ is independently selected from the group consisting of C₁-C₆ alkyl, C₂-C₆ alkenyl, C₁-C₆ heteroalkyl, and C₂-C₆ heteroalkenyl.

As used herein, the term “acyl”, means a group selected from the group consisting of CO-alkyl, CO-alkenyl, CO-heteroalkyl, and CO-heteroalkenyl, each of which may be optionally substituted with one or more groups independently selected from C₁₋₆ alkyl, C₂-6 alkenyl, and halogen.

The term “alkyl” refers to a straight-chain or branched alkyl substituent containing from, for example, 1 to about 36 carbon atoms. Examples of suitable alkyl groups include, but are not limited to, methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl and the like. The number of carbons referred to relates to the carbon backbone and carbon branching but does not include carbon atoms belonging to any substituents, for example the carbon atoms of an alkoxy substituent branching off the main carbon chain.

The term “alkenyl” refers to a straight-chain or branched alkenyl substituent containing from, for example, 2 to about 36 carbon atoms. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, undecenyl, dodecenyl and the like. The number of carbons referred to relates to the carbon backbone and carbon branching but does not include carbon atoms belonging to any substituents, for example the carbon atoms of an alkoxy substituent branching off the main carbon chain.

The term “alkynyl” refers to a straight-chain or branched alkynyl substituent containing from, for example, 2 to about 36 carbon atoms. Examples of suitable alkynyl groups include, but are not limited to, ethynyl, propynyl, butynyl, butadienyl, pentynyl, hexynyl, heptynyl, octynyl, nonynyl, decynyl, undecynyl, dodecynyl and the like. The number of carbons referred to relates to the carbon backbone and carbon branching but does not include carbon atoms belonging to any substituents, for example the carbon atoms of an alkoxy substituent branching off the main carbon chain.

The term “cycloalkyl” refers to a saturated non-aromatic cyclic hydrocarbon. The cycloalkyl ring may include a specified number of carbon atoms. For example, a 3 to 8 membered cycloalkyl group includes 3, 4, 5, 6, 7 or 8 carbon atoms. The cycloalkyl group may be monocyclic, bicyclic or tricyclic. When more than one ring is present the rings are fused together (for example, a bicyclic ring is fused if two atoms are common to both rings). Non-limiting examples may include cyclopropyl, cyclobutyl, cyclopentyl, cyclohexyl and the like.

The term “cycloalkenyl” or “cycloalkene” refers to a cyclic hydrocarbon having at least one double bond, which is not aromatic. The cycloalkenyl ring may include a specified number of carbon atoms. The cycloalkenyl group may be monocyclic, bicyclic or tricyclic. When more than one ring is present the rings are fused together (for example, a bicyclic ring is fused if two atoms are common to both rings). For example, a 5 membered cycloalkenyl group includes 5 carbon atoms. Non-limiting examples may include cyclopentenyl and cyclopenta-1,3-dienyl.

The term “cycloalkynyl” or “cycloalkyne” refers to a cyclic hydrocarbon having at least one triple bond, which is not aromatic. The cycloalkynyl ring may include a specified number of carbon atoms. The cycloalkynyl group may be monocyclic, bicyclic or tricyclic. When more than one ring is present the rings are fused together (for example, a bicyclic ring is fused if two atoms are common to both rings). For example, a 5 membered cycloalkynyl group includes 5 carbon atoms. Non-limiting examples may include cyclopentynyl.

The term “aryl” refers to an aromatic carbocyclic substituent, as commonly understood in the art. It is understood that the term aryl applies to cyclic substituents in which at least one ring is planar and comprises 4π+2 πr electrons, according to Hückel's Rule. Aryl groups may be monocyclic, bicyclic or tricyclic. Examples of aryl groups include, but are not limited to, phenyl, naphthyl and 1,2,3,4-tetrahydronaphthyl. An aryl group may be monocyclic, bicyclic or tricyclic, provided that at least one ring is aromatic. When more than one ring is present the rings are fused together (for example, a bicyclic ring is fused if two atoms are common to both rings).

The term “heteroalkyl” refers to a straight-chain or branched alkyl substituent in which one or more carbon atoms have been replaced by heteroatoms independently selected from N, S and O. It may contain from, for example, 1 to about 36 carbon atoms. For example, between 1 and 4 carbon atoms may be replaced by heteroatoms independently selected from N, S and O. Examples of suitable heteroalkyl groups include, but are not limited to, methoxy, ethoxy, propyloxy, isopropyloxy, and the like. The number of carbons referred to relates to the carbon backbone and carbon branching but does not include carbon atoms belonging to any substituents, for example the carbon atoms of an alkoxy substituent branching off the main carbon chain.

The term “heteroalkenyl” refers to a straight-chain or branched alkenyl substituent in which one or more carbon atoms have been replaced by heteroatoms independently selected from N, S and O. It may contain from, for example, 2 to about 36 carbon atoms. For example, between 1 and 4 carbon atoms may be replaced by heteroatoms independently selected from N, S and O. The number of carbons referred to relates to the carbon backbone and carbon branching but does not include carbon atoms belonging to any substituents, for example the carbon atoms of an alkoxy substituent branching off the main carbon chain.

The term “heteroalkynyl” refers to a straight-chain or branched alkynyl substituent in which one or more carbon atoms have been replaced by heteroatoms independently selected from N, S and O. It may contain from, for example, 2 to about 36 carbon atoms. For example, between 1 and 4 carbon atoms may be replaced by heteroatoms independently selected from N, S and O. The number of carbons referred to relates to the carbon backbone and carbon branching but does not include carbon atoms belonging to any substituents, for example the carbon atoms of an alkoxy substituent branching off the main carbon chain.

The term “heterocyclic” or “heterocyclyl” as used herein, refers to a cycloalkyl or cycloalkenyl group in which one or more carbon atoms have been replaced by heteroatoms independently selected from N, S and O. For example, between 1 and 4 carbon atoms in each ring may be replaced by heteroatoms independently selected from N, S and O. The heterocyclyl group may be monocyclic, bicyclic or tricyclic in which at least one ring includes a heteroatom. When more than one ring is present the rings may be fused together (for example, a bicyclic ring is fused if two atoms are common to both rings). Each of the rings of a heterocyclyl group may include, for example, between 5 and 7 atoms. Examples of heterocyclyl groups include tetrahydrofuranyl, tetrahydrothiophenyl, pyrrolidinyl, pyrrolinyl, dithiolyl, 1,3-dioxanyl, dioxinyl, piperidinyl, piperazinyl, morpholinyl, thiomorpholinyl, pyranyl, 1,4-dithiane, and decahydroisoquinoline. In one embodiment, heterocyclyl may be optionally substituted by ═O.

The term “heteroaryl”, as used herein, refers to a monocyclic, bicyclic or tricyclic ring of up to 7 atoms in each ring, wherein at least one ring is aromatic and said at least one ring contains from 1 to 4 heteroatoms selected from the group consisting of O, N and S. When more than one ring is present the rings are fused together (for example, a bicyclic ring is fused if two atoms are common to both rings). Consideration must be provided to tautomers of heteroatom containing ring systems containing carbonyl groups, for example, when determining if a ring is a heterocyclyl or heteroaryl ring. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having four heteroatoms (e.g., tetrazoles); 6-membered heteroaryls with one heteroatom (e.g., pyridine, quinoline, isoquinoline); 6-membered heteroaryls with two heteroatoms (e.g., pyridazines, cinnolines, phthalazines, pyrazines, pyrimidines, quinazolines, quinoxalinone, quinazolinone); 6-membered heteroaryls with three heteroatoms (e.g., 1,3,5-triazine); and 6-membered heteroaryls with four heteroatoms. Examples of heteroaryl include thiophene, benzothiophene, benzofuran, benzimidazole, benzoxazole, benzothiazole, benzisothiazole, furan, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, and phenoxazine. Further exemplary heteroaryl groups may include, for example, indoline or 2,3-dihydrobenzofuran. In one embodiment, heteroaryl may be optionally substituted by ═O.

The term “terpenoid”, or “terpenoidyl” as used herein, refers to an organic chemical derived from one or more isoprene units. Steroids are a sub-class of terpenoid.

The term “steroid”, or “steroidyl” as used herein, refers to a carbocyclic moiety comprising a backbone having four fused rings having the following arrangement:

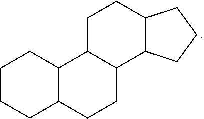 [SMILES: C1CCC2C(C1)CCC1C3CCCC3CCC21]

The rings may be saturated carbocycles, or they may comprise one or more double bonds. One or more of their ring carbon atoms may be substituted with one or more R¹⁵ groups, wherein R¹⁵ is selected from the group consisting of═O, ═S, OH, SH, NH₂, C₁-C₁₂ alkyl, C₂-C₁₂ alkenyl, C₂-C₁₂ alkynyl, aryl, C₁-C₁₂ heteroalkyl, C₂-C₁₂ heteroalkenyl, C₂-C₁₂ heteroalkynyl, and heteroaryl. A steroid or steroidyl group may be bonded to another group through one or more substituents attached to its backbone. For example, cholesterol/cholesteryl or cholestanol/cholestanyl may be connected to another moiety through its OH substituent group.

Whenever a range of the number of atoms in a structure is indicated (e.g., a C₁-C₁₂, C₁-C₆ alkyl, etc.), it is specifically contemplated that any sub-range or individual number of carbon atoms falling within the indicated range also can be used. Thus, for instance, the recitation of a range of 1-12 carbon atoms (e.g., C₁-C₁₂), 1-6 carbon atoms (e.g., C₁-C₆) as used with respect to any chemical group (e.g., alkyl, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

As used herein, “halo” refers to a halogen atom, especially F, C₁ or Br; more especially F or C₁; most especially F.

As used herein, the term “optionally substituted” means that any number of hydrogen atoms on the optionally substituted group are replaced with another moiety. Unless defined otherwise, said moiety is independently selected from the group consisting of C₁-C₁₂ alkyl (or C₁-C₆ alkyl), C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹²—CONH—R¹³, R¹²—NHCO—R¹³, R¹²-CSNH—R¹³, R¹²—NHCS—R¹³, R¹²—CO—R¹³, =0, ═S, cyano, CF₃, and halogen;
wherein:
   R¹² is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
   R¹³ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and hydrogen.

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### EXAMPLES

### Synthesis of Compound 1

Overall synthesis

The synthesis of Compound 1 was performed according to the five-stage synthesis outlined in Scheme 1. Each stage of the synthesis is discussed in further detail in the following sections.

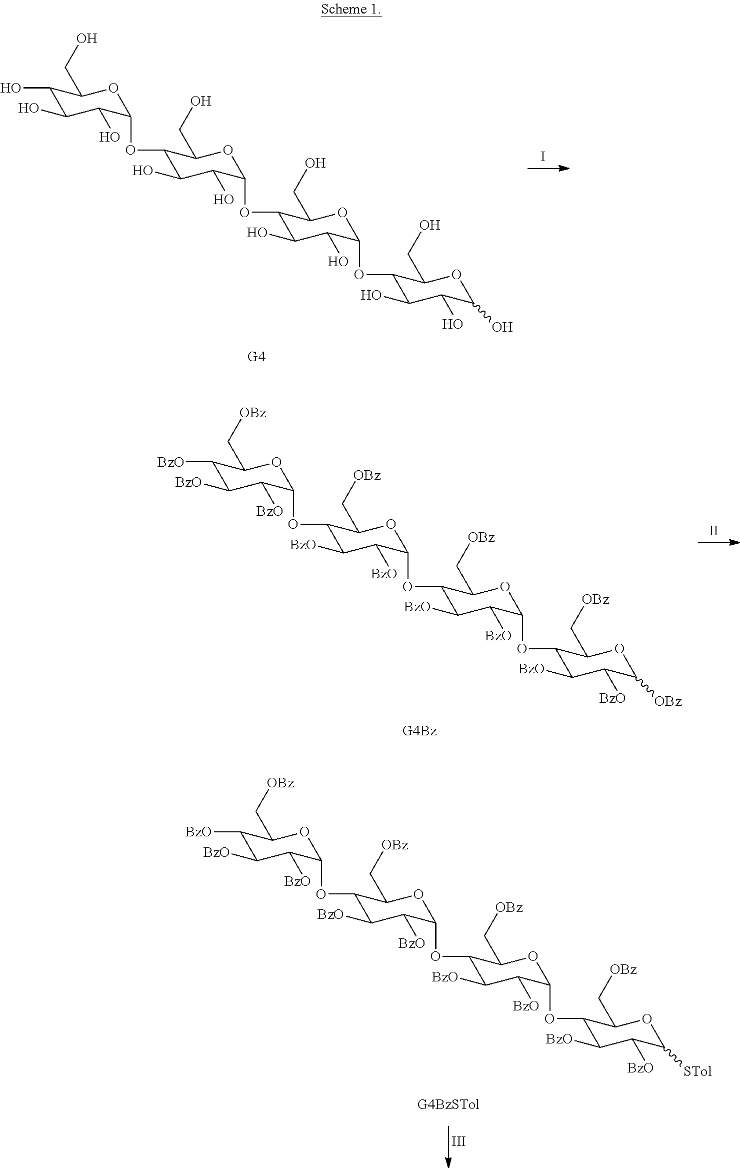

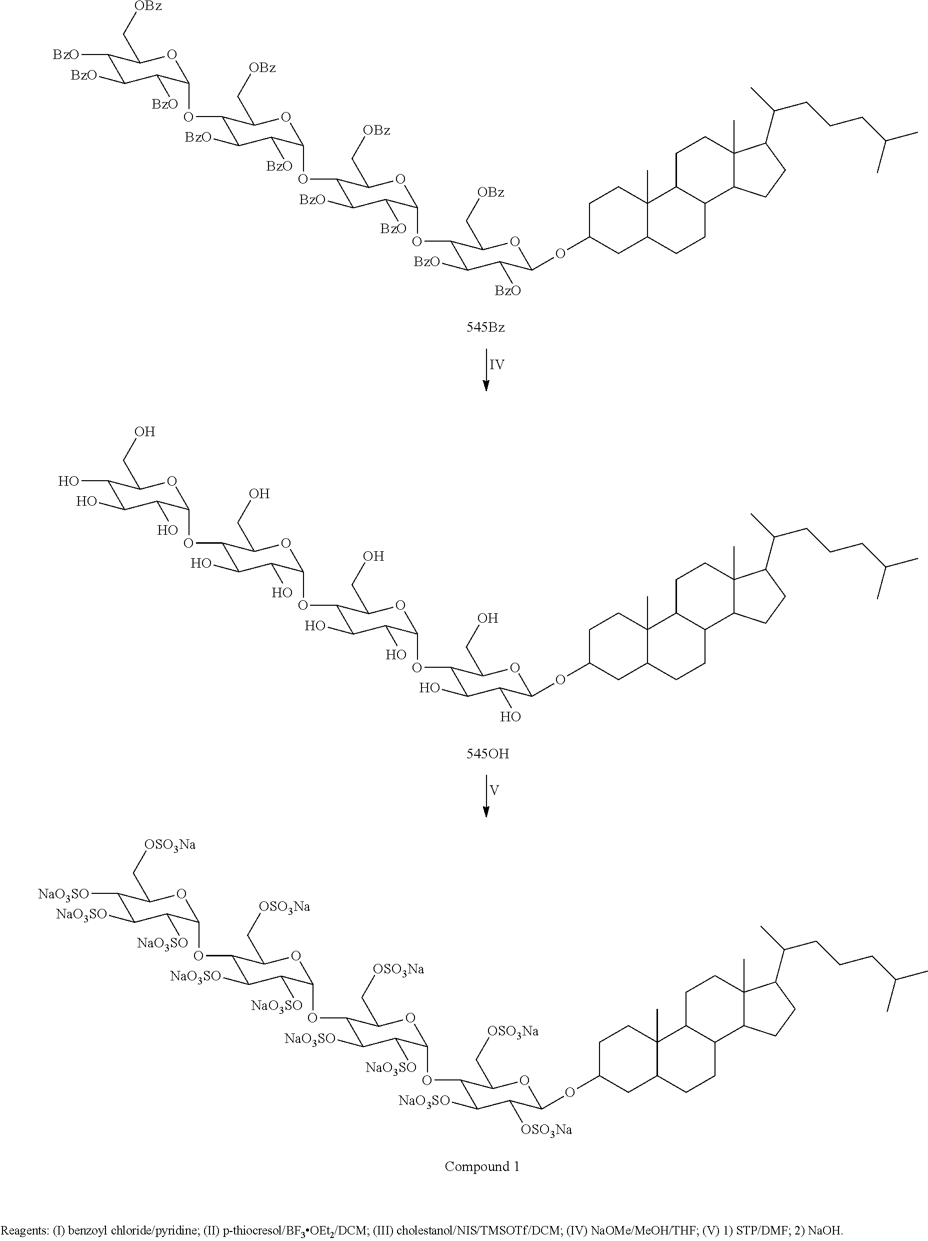

### Stage 1: Synthesis of G4Bz

The synthesis of G4Bz from G4 is outlined in Scheme 2 below. Briefly, the hydroxyl groups of G4 (maltotetraose) were benzoylated using benzoyl chloride in pyridine.

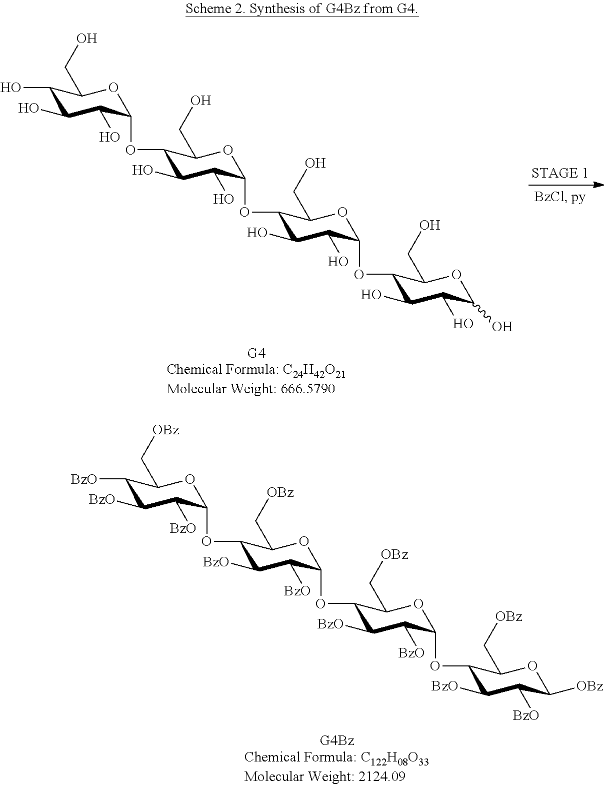

### Materials

| | | |
|---|---|---|
| Reagents | Quantity (wts.) | Quantity (g) |

| | | | | |
|---|---|---|---|---|
| G4 syrup (~50% w/w by | 1 | wt. | 200 | g |
| dry weight) | | | | |

| | | |
|---|---|---|
| Pyridine | 5.84 wts. + 2.0 wts | 1168 g + 400 g |

| | | | | |
|---|---|---|---|---|
| Benzoyl chloride | 2.66 | wts. | 532 | g |

| | | |
|---|---|---|
| 2-Propanol | 11.88 wts. + 2.0 wts | 2376 g + 400 g |

| | | | | |
|---|---|---|---|---|
| Ethyl acetate | 7 | wts. | 1400 | g |
| Water | 1.0 | wts. | 200 | g |
| 10% aq. NaCl | 2 × 5.0 | wts | 2 × 1000 | g |
| 2M aq. HCl | 3 × 5.0 | wts | 3 × 1000 | g |
| TBME-Heptane (9:1 v/v) | 2.0 | wts | 400 | g |

### Procedure

1. A solution of G4 syrup (comprising maltotetraose at ˜50% (w/w) by dry weight) in pyridine (200 g in 1168 g) in a reaction vessel was azeotropically dried by distilling ca. 3.0 wts. of solvent at ˜ 100° C./250 mbar (˜600 mL); the solution was dried to a moisture content of <133% (w/w) as determined by Karl Fischer (KF) titration.

2. Pyridine (400 g) was charged into the reaction vessel and the solution was heated to 90° C.

3. Benzoyl chloride (532 g) was added slowly, maintaining the reaction at 100-110° C.

4. The reaction was stirred under Argon at 105-115° C. for 16-20 h.

5. The reaction was cooled to 90° C., then quenched by the addition of H₂O (200 g); maintaining the reaction mixture at <110° C. during the quench.

6. The reaction was cooled to 30° C. and ethyl acetate (EtOAc) (1400 g) was charged into the reaction vessel.

7. Brine (10% aq. NaCl, 1000 g) was charged into the reaction vessel and the phases mixed for >5 minutes. The aqueous phase was separated and drained.

8. 2M aq. HCl (1000 g) was charged into the reaction vessel and the phases were mixed for >5 minutes. The aqueous phase was separated and drained. The 2M aq. HCl (1000 g) washes were repeated until the pH of the aqueous phase was <2.

9. Brine (10% aq. NaCl, 1000 g) was charged into the reaction vessel and the phases were mixed for >5 minutes. The aqueous phase was separated and drained.

10. The reaction mixture was concentrated to approximately 4-5 volumes by distilling EtOAc at ˜ 70° C./200 mbar.

11. The crude solution of G4Bz in EtOAc was cooled to 20° C. and drained into a bottle (labelled “Crude G4Bz in EtOAc”). The crude solution was <5 wts. EtOAc to ensure that the material did not form a gummy aggregate but not too concentrated as it would tend to form more granular solids during precipitation.

12. Isopropyl alcohol (WA) (11.88 wts, 2376 g) was charged to the reactor and cooled to 15° C.

13. “Crude G4Bz in EtOAc” was charged slowly to the reactor using a peri-pump. The G4Bz solution was charged such that it easily dissipated into the WA and did not form clumps. The resulting suspension was stirred at 15° C. for 30-60 minutes.

14. The suspension was drained portion-wise from the reactor and filtered via vacuum filtration through a filter cloth.

15. The filter cake was washed with 1×2.0 wts of IPA (400 g) and 1×2.0 wts of (methyl tert-butyl ether) TBME-Heptane (1:9 w/w, 400 g).

16. The solids were transferred into a tared flask and dried to a constant mass by rotary evaporation.

### Step 1: Drying of Maltotetraose Syrups

Maltotetraose is available as either a high-purity solid, or as various oligosaccharide mixtures in the form of syrups containing ˜25% water. Whether a >70% purity (on dried basis) syrup is used, or the >50% syrup as used in the present example, the water must be removed as it interferes with the addition of the protecting groups in the next step.

The syrups may be dried by lyophilization. Other methods of drying such as spray drying or acetone precipitation may be used. The pyridine azeotrope method was successfully employed on scale-up (˜2 kg of >50% syrup) for production of API (active pharmaceutical ingredient) for clinical trial use. The residual water after the pyridine azeotrope step was measured using Karl Fischer titration. Experiments on spiking with known quantities of water established that >2% residual water leads to incomplete benzoylation (Step 4). Residual water at 1.35% gave a complete reaction.

### Step 2: Charging Additional Pyridine

Pyridine was added back in the reaction vessel after the azeotropic distillation so that the benzoylation reaction was performed in about 5 weights of pyridine in total. This prevented precipitation of solid during the benzoylation that otherwise could clump up and interfere with the stirring.

### Steps 2-4: Benzoylation Conditions

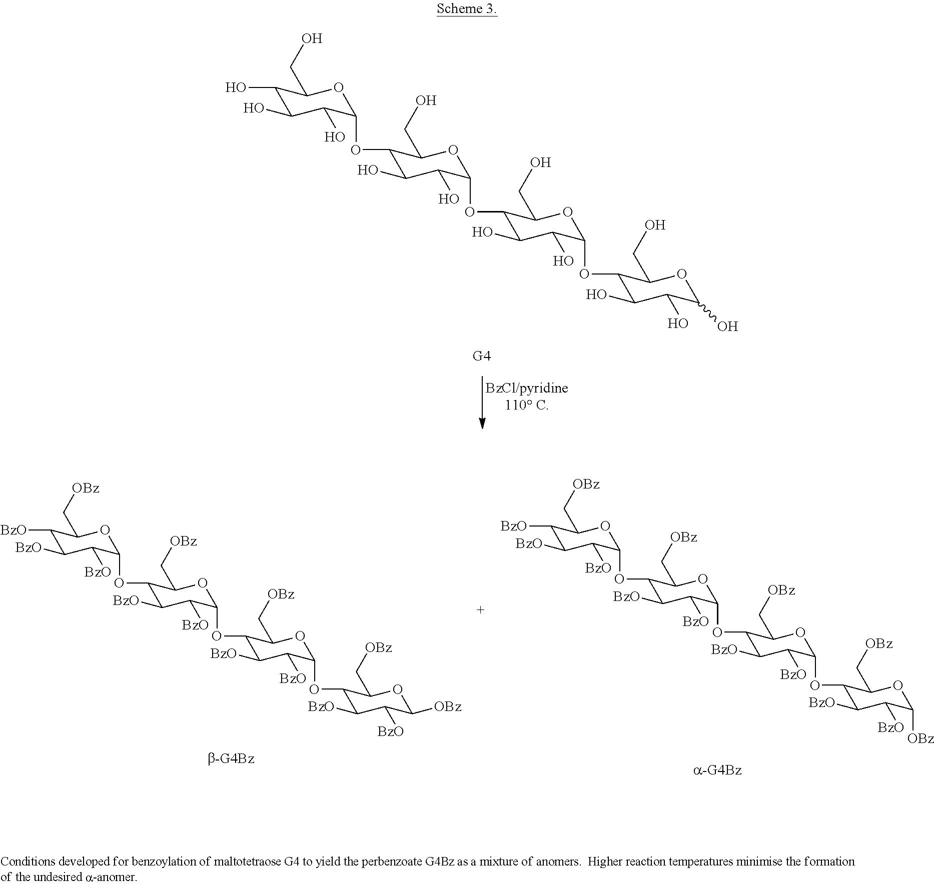

Benzoylation of alcohols is known in the art. However, the α/β-selectivity of the protection chemistry (shown in Scheme 3) as distinct from the α/β-selectivity of the glycosylation step, is important, because only the β-anomer of perbenzoate G4Bz is observed to react in the next chemical step and therefore the unreacted α-anomer is an undesired impurity. Formation of the undesired α-anomer may be minimized by performing the reaction at elevated temperature (typically greater than 100° C.).

### Step 5: Quench

The unreacted benzoyl chloride was quenched at the end of the reaction. This prevented further uncontrolled/undesired reaction during workup, and prevents exposure of workers to this hazardous compound. The reaction was quenched with water so that the benzoic acid by-product could be removed in the aqueous workup. Quenching with IPA also worked, with the resulting isopropyl benzoate by-product removed in the precipitation/wash steps.

### Steps 7-9: Acid Wash

The isolation of the product G4Bz from the reaction mixture was based on washing to remove pyridine residues, followed by precipitation using a mixture of solvents. Suitable conditions were those that controlled the level of residual pyridine or other bases, and water, in the product, since these can interfere with the subsequent step, while leaving the product as a filterable solid for easy isolation and handling. Levels of pyridine in the product were measured using HPLC analysis, or by UV/Visible spectroscopy (e.g. at a wavelength of 290 nm). Residual water was quantified e.g. by Karl Fischer titration.

An initial brine wash (Step 7) was used to reduce the number of acid washes required to remove all of the pyridine, from ˜5 to ˜3 washes, and prevented the formation of emulsions where aqueous and organic layers were difficult to separate. The final brine wash (Step 9) reduced residual HCl which is corrosive.

Continuing acid-washes until the pH of the aqueous layer remains under 2 yields product (after final drying) with a preferred pyridine content of <1% by HPLC. Higher amounts can be tolerated in the following Stage e.g. <5% pyridine by HPLC, but require that additional BF₃ is added to compensate.

### Steps 10-13: Precipitation Conditions

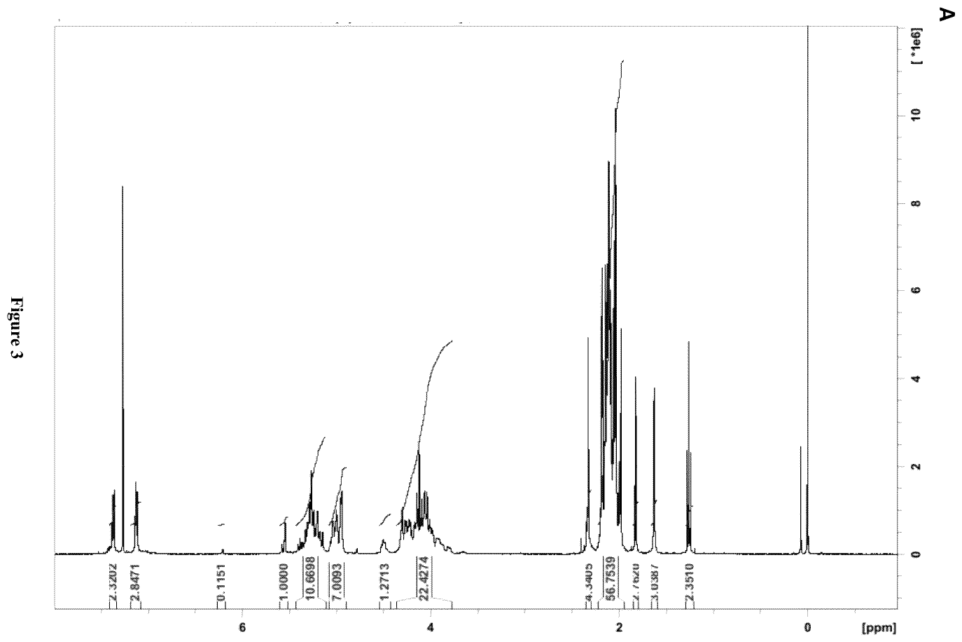

The perbenzoylated products throughout the example syntheses of Compound 1 and of Compound 4 could be precipitated in a range of forms from a viscous oil to a glassy solid. Under suitable conditions, the products can be reliably precipitated in the form of glassy, amorphous beads (see, e.g., FIG. 2) which are highly suitable for isolation by vacuum filtration. In general, suitable precipitation conditions begin with a solution of the intermediate in a strong solvent, which is added with vigorous stirring to an anti-solvent (“reverse addition”). Most alcohols are suitable as an anti-solvent, with isopropanol most preferred for reasons of efficacy, safety and cost; aliphatic hydrocarbons such as heptane are also suitable. Suitable strong solvents include ethyl acetate, ethers especially THF, and aromatics such as toluene or pyridine. Dichloromethane should be avoided, since even small quantities interfered with the precipitation and resulted in the formation of an oily product.

The steps above demonstrate removal of pyridine by an acid-wash, followed by precipitation incorporating only minimal quantities of water as retained in the organic layer after washing. This method is preferred since it minimises the amounts of both residual pyridine and residual water that must be removed from the final product by drying (Step 16). Alternatively, bulk pyridine may be removed in the precipitation process. In this case, the presence of large quantities of pyridine in the strong solvent required large quantities of water in the anti-solvent for correct formation of the filterable solid. The resulting filter cake contained appreciable quantities of both pyridine and water, requiring extended drying time (days). The acid wash procedure, while adding repeated wash steps, yielded a precipitated product that was much more easily dried, and thus is more efficient overall.

### Step 14: Isolation by Filtration

On kilogram scale, the filter cakes former in the filtration step were noted to be compressible solids. This meant that too high a pressure differential during filtration, whether by application of vacuum below the filter plate or air/nitrogen above, caused the cakes to compress and greatly slow the flow rate of the filtrate. Ideally, the bulk of the mother liquor was allowed to proceed under gravity at atmospheric pressure, then application of pressure was used for the final deliquoring/drying step.

### Stage 2: Synthesis of G4BzSTol

The synthesis of G4BzSTol from G4Bz is outlined in Scheme 4 below. Briefly, the anomeric group of G4Bz is thiolated using para-thiocresol and BF₃·OEt₂ in dichloromethane.

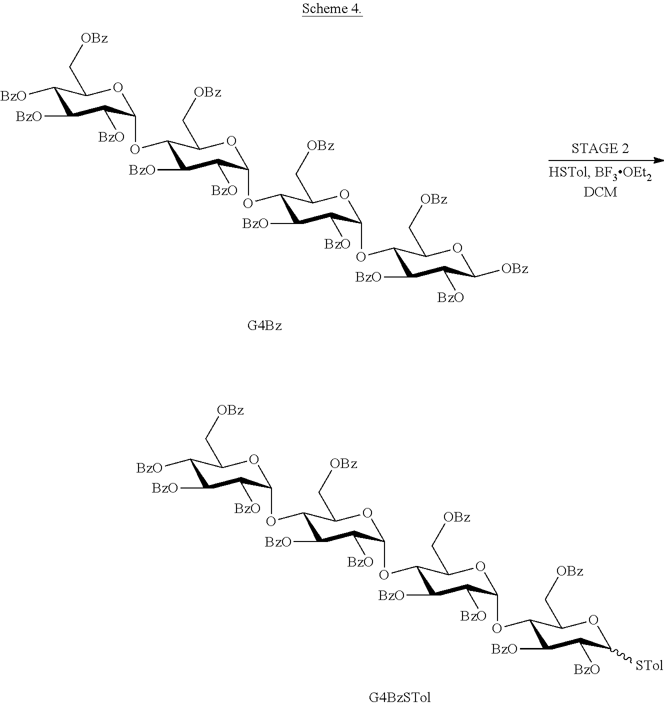

### Materials

| Reagents | Quantity (wts.) | | Quantity (g) | |
|---|---|---|---|---|
| G4Bz | 1 | wt. | 275 | g |
| Thiocresol | 0.09 | wt. | 24.75 | g |
| DCM | 2.91 | wts. | 800.25 | g |
| BF₃•Et₂O | 0.12 | wt. | 34 | g |
| Et₃N | 0.10 | wt. | 26.62 | g |
| Ethyl acetate | 5.82 | wts. | 1600 | g |
| 10% aq. KOH/5% aq. Na₂S₂O₃ | 2 × 5.0 | wts. | 2 × 1375 | g |
| 10% aq. NaCl | 2 × 5.0 | wts. | 2 × 1375 | g |
| Water | 4.0 | wts. | 1100 | g |
| Propanol | 7.02 | wts. | 1939 | g |
| Heptane | 4.72 | wts. | 1298 | g |

### Procedure

1. G4Bz (1 wt.) and thiocresol (0.09 wt.) were charged to the reactor and dissolved in DCM (2.65 wts). The solution was then warmed to 35° C.

2. BF₃·OEt₂ was charged to the reactor slowly, maintaining the reaction at <

40° C. during the addition. A line rinse was performed with DCM (0.26 wts).

3. The reaction was stirred at 35° C.±5° C. and monitored by HPLC for reaction completion (NMT (not more than) 1.0% G4Bz).

4. The reaction was cooled to 20° C.±5° C. and quenched with the addition of Et₃N (0.10 wts.) in EtOAc (5.82 wts.), maintaining the reaction temperature at <40° C. during the quench.

5. The organic phase was washed with the following: 1×5 wts. 10% aq. NaCl, 2 ×5 wts. 10% aq. KOH/5% aq. Na₂S₂O₃, 1×5 wts. 10% aq. NaCl.

6. The aqueous phases were separated and drained from the reactor. The organic phase was concentrated to 2.5 volumes by vacuum distillation and then drained from the reactor.

7. Water (4 wts.) and WA (7 wts.) were charged to the reactor and cooled to 15° C. 5° C.

8. The organic phase was charged slowly into the reactor, ensuring that it dissipated quickly into the WA—water solution upon contact.

9. The suspension was stirred at 15° C.±5° C. for 1 hour±30 minutes.

10. The suspension was drained from the reactor and filtered through a filter cloth (105 μm PP(polypropylene)) via vacuum filtration. The filter cake was washed with WA (2 wts.).

11. Heptane (4.72 wts.) was charged to the reactor and cooled to 15° C.±5° C. The filter cake from step 10 was charged to the reactor, and the suspension was stirred for 30 minutes±15 minutes.

12. The suspension was drained from the reactor and filtered through a filter cloth (105 μm PP) via vacuum filtration. The filter cake was washed with heptane (2 wts.).

13. The solids were transferred to a tared evaporating flask and dried to a constant mass.

### Step 2: Conditions for Formation of the Thioglycoside

Significant optimization of the conditions for formation of thioglycoside G4BzSTol was performed, and the above process is considered suitable for scale-up. Boron trifluoride was used as a Lewis acid catalyst, although a skilled worker would understand that this may be substituted with other promoters.

Somewhat surprisingly, the presence of ethereal solvents has a significant effect on reaction rate. The use of boron trifluoride diethyl ether complex in THE solvent completely inhibits the reaction, while the same complex inDCM solvent is successful. Use of boron trifluoride acetic acid complex in DCM solution, where no ethers are present, gives a large enhancement of both the desired reaction as well as side reactions, such that the starting material G4Bz is consumed within a few minutes, and impurities begin to accumulate. Accordingly, it may be possible to modulate the reactivity by the addition of ethers.

The use of triethylamine as a Lewis base to quench the reaction on completion neutralised the Lewis acidic boron trifluoride and enabled the reaction mixture to be held overnight if required, before the workup was commenced, without further reaction leading to the formation of impurities.

### Step 1: Stoichiometry of Thiocresol

Although a larger excess of thiocresol helped ensure that the reaction was driven to completion, this excess reagent needed to be purged during the reaction workup or else it would interfere with the glycosylation reaction (Stage 3). Charging the reactor with 0.09 weights of thiocresol resulted in an easier workup compared with using a larger excess (e.g. 0.11 weights) while still giving complete conversion (<1% starting material G4Bz remaining by HPLC).

Step 2: Stoichiometry of BF₃·OEt₂

An excess of boron trifluoride helped to drive the reaction to completion. However, too much BF₃·OEt₂ resulted in problems with the aqueous workup. Quenching boron trifluoride in water can yield metaboric acid which is only slightly soluble in water, interfering with the separation of aqueous and organic phases in the extraction.

The conditions chosen represents a slight excess of BF₃ with respect to thiocresol (e.g. 0.2 molar equivalents excess). A larger excess (e.g. 0.8 molar equivalents in excess) was problematic.

### Step 5: Wash Procedure

The initial NaCl wash removed BF₃·OEt₂ as a suitable water soluble boron derivative. If significant amounts of boron were present in the subsequent KOH wash, the formation of troublesome metaboric acid seemed to be favoured.

The wash with 10% NaOH was important to remove excess thiocresol, as otherwise the subsequent glycosylation stage did not go to completion. A weaker base (sodium bicarbonate) did not completely remove the thiocresol residues by itself. Thiosulfate was added as a reducing agent to clean up oxidised forms of thiocresol (which was important for the subsequent glycosylation stage where free iodine was present as an oxidising agent, but also was included in this step also).

### Steps 11-12: Drying Procedure

Residual water used in the precipitation step was removed to prevent it from quenching the TMSOTf promoter in the subsequent glycosylation reaction (Stage 3). Slurrying the precipitated product in heptane (Step 11) yielded a slightly different, more powdery form that could more easily be dried (Step 12). Note that simply washing the filter cake on the filter with heptane was not sufficient, and the slurrying procedure was required to properly alter the solid form.

Stage 3: Synthesis of545Bz

The synthesis of 545Bz from G4BzSTol is outlined in Scheme 5 below. Briefly, cholestanol is glycosylated using G4BzSTol, NIS and TMSOTf in dichloromethane.

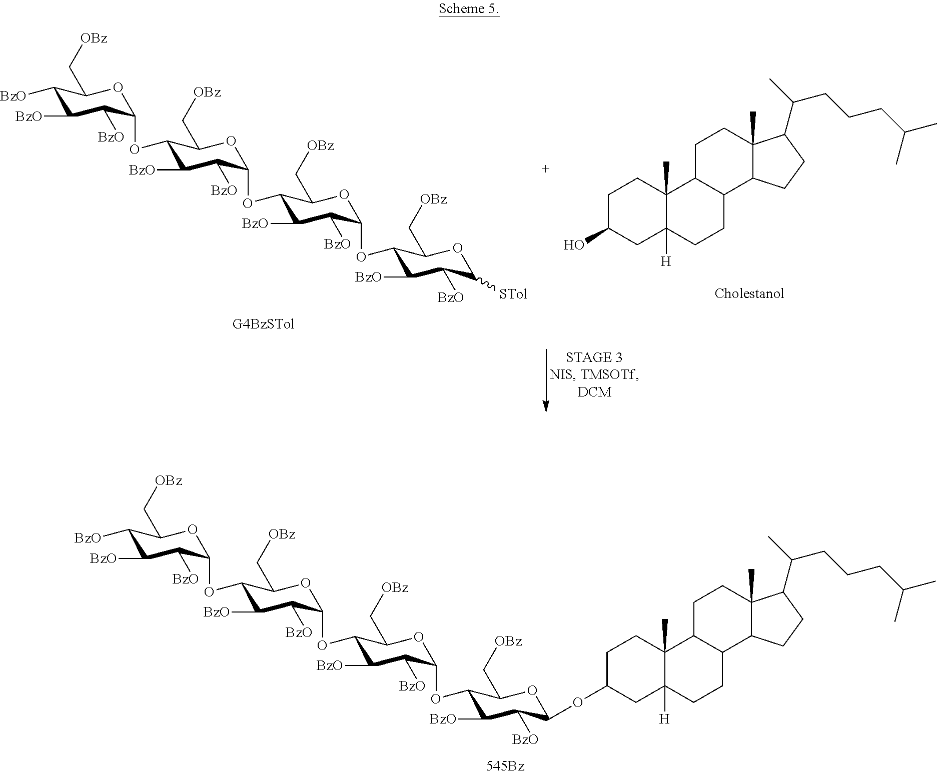

### Materials

| Reagents | Quantity (wts.) | | Quantity (g) | |
|---|---|---|---|---|
| G4BzSTol | 1 | wt. | 250 | g |
| 5α-cholestan-3β-ol | 0.20 | wt. | 50.27 | g |
| DCM | 7.96 wts. + 0.10 wt. | | 1990 g + 25 g | |
| NIS | 0.21 | wt. | 52.91 | g |
| TMSOTf | 0.07 | wt. | 16.25 | g |
| 10% aq. KOH/5% aq. Na₂S₂O₃ | 2 × 5.0 | wts. | 2 × 1250 | g |
| 5% aq. NaHCO₃ | 1 × 5.0 | wts. | 1 × 1250 | g |
| 10% aq. NaCl | 1 × 5.0 | wts. | 1 × 1250 | g |
| EtOAc | 2.70 | wts. | 675 | g |

| | | | |
|---|---|---|---|
| Heptane | 13.70 | wts. | 3425 |

### Procedure

1. A solution of G4BzSTol (1.0 wts) and 5α-cholestan-3β-ol (0.20 wts) in DCM (7.96 wts) was stirred under N₂ for 30-60 min.

2. The mixture was cooled to 5° C.±5° C. and NIS (0.21 wts.) was added. TMSOTf (0.07 wts) was added slowly, maintaining the reaction temperature at <10° C. during addition, followed by a DCM line rinse (0.10 wts).

3. The mixture was stirred at 5° C.±5° C. for 2 h±15 min, until HPLC confirmed that G4BzSTol was NMT 4.0%.

4. The reaction was quenched by adding a solution of 10% aq. KOH/5% aq. Na₂S₂O₃ (5 wts.), maintaining the reaction temperature at <20° C. during addition.

5. The phases were separated, and the organic phase was washed with the following: 1×5 wts. 10% aq. KOH/5% aq. Na₂S₂O₃ solution, 1×5% aq. NaHCO₃, 1×10% aq. NaCl.

6. A solvent swap into EtOAc was performed by adding EtOAc (2.7 wts) then concentrating the solution down to 2.5 volumes by vacuum distillation at 45° C.

7. The solution of 545Bz in EtOAc was added slowly to heptane (13.7 wts) at 15-25° C. to precipitate the product.

8. The product was filtered off, washed with heptane (2 wts.) and dried to constant mass by rotary evaporation at 45° C.

### Steps 1-2: Reaction Stoichiometry

An excess of cholestanol was used, otherwise the reaction did not go to completion and the hemiacetal side-product G4BzOH was produced. A slight excess of NIS was also required, otherwise the conversion would stall and unreacted thioglycoside G4BzSTol remain. The TMSOTf promoter was used catalytically (i.e. sub-stoichiometrically). The amount of solvent DCM was important; at Step 1 the mixture forms a gel-like suspension that can be difficult to stir. Adding more DCM allows efficient stirring, but surprisingly once the TMSOTf is added the suspension dissolved anyway (possibly the hydroxyl group of the cholestanol was converted to a TMS ether).

### Steps 2-3: Reaction Temperature

The reaction conditions employed required careful optimisation to ensure the reaction went to completion. The conditions used maximized the formation of the desired β-anomer β545Bz (Scheme 6), including conducting the reaction at ice temperature and monitoring the reaction by HPLC. The desired β-anomer β545Bz appears to be the initially formed kinetic product, which rearranges to the thermodynamically favoured α545Bz upon prolonged reaction.

Lower temperatures favoured the formation of the correct β-anomeric configuration, 5° C. (chilled water) was convenient for large-scale manufacture.

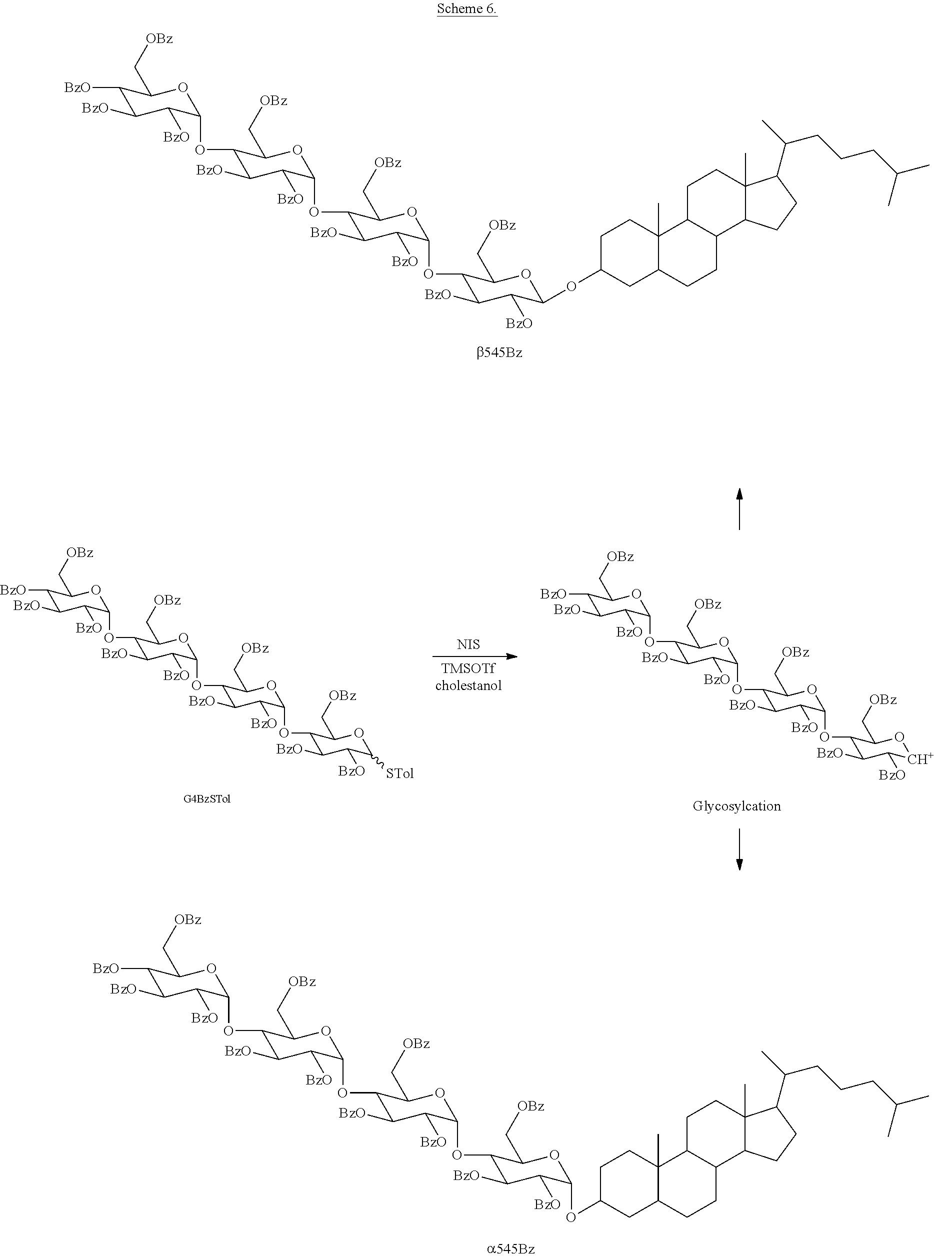

### Step 4-5: Quench Wash Solutions

Thiosulfate was used to remove free iodine (from the reaction of NIS) by reduction to water-soluble iodide, and also to reduce any disulfide-linked thiocresol dimers back to the corresponding thiol, so that they could be removed by washing with KOH base.

### Step 6-7: Precipitation

Small amounts of DCM interfered with formation of the easily-filterable solid form, and accordingly DCM was replaced with EtOAc. Addition into the correct quantity of non-solvent (heptane here, but IPA would also work) resulted in product precipitation. Small amounts of water seem to be necessary for formation of the solid form, so no drying of the EtOAc solution was performed.

### Step 8: Heptane Slurry

Once correctly precipitated, the solid product was treated by slurrying it with heptane in order to convert it to a finer, more powdery solid for easier drying.

### Stage 4: Synthesis of 5450H

Global deprotection of perbenzoate 545Bz was accomplished using standard Zemplen transesterification with methanol, catalysed by methoxide as shown in Scheme 7. The deprotection chemistry has a major effect on the properties of the molecule: perbenzoate 545Bz is insoluble in water, while polyol 545OH is soluble in water but poorly soluble in almost all organic solvents. Purification or manipulation of polyol 545OH therefore is easiest in aqueous solution, but the water must then be removed as it interferes with the final sulfation step.

Previously disclosed workups for this reaction included quenching the methoxide catalyst using resin-supported acid, then filtering and evaporating the reaction mixture to dryness. However, both the use of resins and evaporation to dryness may be impractical on scale-up as they can lead to cleaning issues and safety issues, respectively. For example, resin beads are typically insoluble in standard cleaning solvents and as such can be difficult to remove from reactor vessels, filtration equipment and pipes. Further, the use of THF, which can be contaminated with peroxides, may result in an explosion if this solvent is evaporated to dryness.

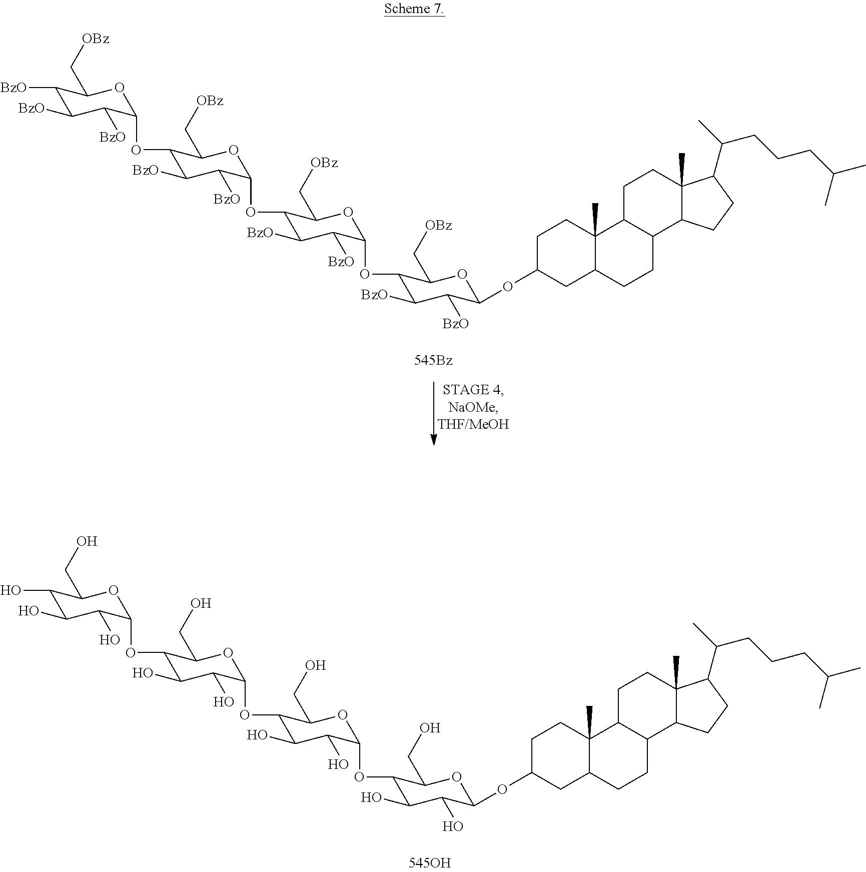

### Materials

| Reagents | Quantities (wts.) | | Quantities (g) | |
|---|---|---|---|---|
| 545Bz | 1 | wt | 200 | g |
| THF | 1.78 wts (2 vol) | | 356 | g |
| MeOH | 3.16 wts (4 vol) | | 632 | g |
| 25% NaOMe in MeOH | 0.31 | wts | 62.0 | g |

| | | | |
|---|---|---|---|
| AcOH | As req. | ~19 | g |

| | | |
|---|---|---|
| EtOAc | 9.02 wts + 2 × 0.9 wts | 1804 g + 2 × 180 g |

### Procedure

1. A suspension of 545Bz in THE (1.78 wts) and MeOH (3.16 wts) was treated with 25% w/w NaOMe in MeOH (0.31 wts) under N₂.

2. The mixture was stirred at 50-60° C. for 3-3.5 h then cooled to 15-25° C. and stirred for a further 16-24 h.

3. The reaction pH was adjusted to 6-8 by the addition of glacial AcOH.

4. The mixture was distilled under vacuum at 45° C. to 2.0 volumes.

5. The mixture was added slowly to EtOAc (9.02 wts) at 15-25° C. to precipitate the product.

6. The suspension was filtered, and the filter cake was washed with EtOAc (2×0.9 wts).

7. The product was dried to a constant mass at 45° C. under vacuum to give crude product (60% 545OH by mass).

Purification by ultrafiltration and precipitation

8. Crude 545OH (1 wt calculated from active mass) in water (20 wts) was filtered through a 0.22 μm filter and charged to the retentate tank.

9. Ultrafiltration/diafiltration in water was performed using a 30 kDa MWCO filter (160 wts, 8 diavolumes).

10. The product solution was concentrated to 8.0 volumes then slowly added to acetonitrile (7.37 wts. with respect to (wrt) the aqueous solution; 59 wts. wrt the contained 5450H).

11. The precipitated product was isolated using a filter under N₂ and was washed with acetonitrile (3.14 wts)/EtOAc (3.61 wts), and then EtOAc (3.61 wts).

12. The solid was slurried in EtOAc (3.16 wts) and then filtered. The filter cake was washed with EtOAc (3.16 wts×2).

13. The product was dried to constant mass at 45° C. under vacuum.

### Step 1: Solvent Ratio

The polarity and solubility of the product changes significantly during the debenzoylation reaction (when compared with the starting material). The perbenzoate 545Bz is soluble in THE (2 volumes) but insoluble in MeOH, while the polyol 545OH is slightly soluble in MeOH, but more soluble when THE is added. The ratio of THF:MeOH is therefore important to balance solubility of both starting material and product, and maintain adequate stirring during reaction.

### Step 5: Precipitation Washing

Although the polyol 545OH was poorly soluble in most organic solvents, the form it takes on completion of debenzoylation could be converted to a solid by precipitation with MeCN, optionally mixed with EtOAc. This solid form was suitable for isolation by filtration under inert atmosphere, but was hygroscopic and not suitable for storage. Slurry washing with EtOAc converted the solid to a non-hygroscopic form that could be more easily handled.

### Purification by Diafiltration Ultrafiltration

The inventors of the present invention have surprising found that polyol 545OH is strongly retained by a 30 kDa MWCO regenerated cellulose membrane, allowing low-molecular weight contaminants to pass through and be removed. As the molecular weight of 545OH is only 1037.23 Da, such strong retention by a large pore-size membrane was unexpected.

Without being bound by theory, the amphiphilic nature of the polyol 545OH may cause it to form large micellular structures in aqueous solution that enable it to be retained by the 30 kDa MWCO membrane.

### Removal of Non-Coupled Oligosaccharides and Low-Molecular Weight Impurities

The major benefit of ultrafiltration as a purification technique is that the purification is dependent on size, rather than on the chemical nature of each impurity. Only molecules which form part of the micellar structure are retained by the membrane, and all other components below the molecular weight cut-off are removed, whether they be solvents, or inorganic or organic impurities. In particular, the α-anomer of perbenzoate G4Bz is chemically unreactive, and is carried through the glycosylation reaction unchanged. This impurity is converted back to maltotetraose G4 in the global deprotection (Scheme 8), and is efficiently removed by ultrafiltration as it lacks a cholestanol group, and hence is not incorporated into micelles.

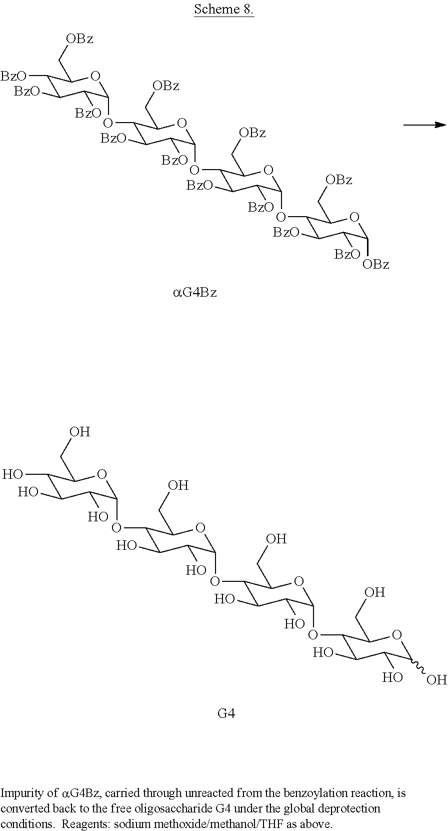

### Stage 5: Synthesis of Compound 1

The synthesis of Compound 1 uses a combination of sulfur trioxide pyridine complex (˜3 eq. per hydroxyl group) in DMF solution as outlined in Scheme 9.

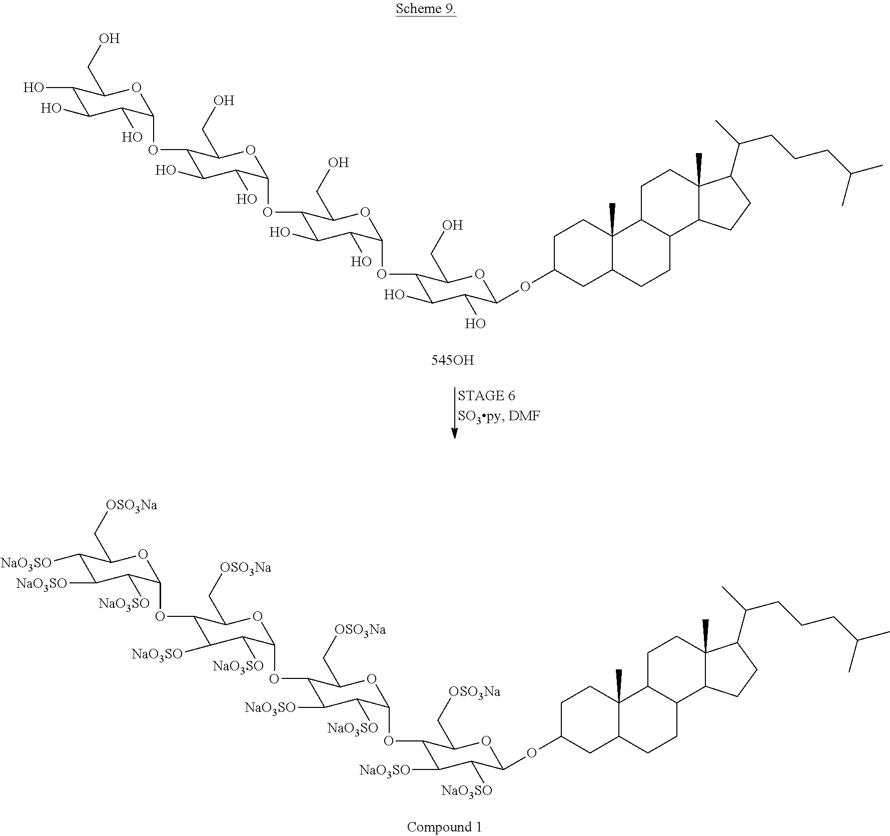

### Materials

| Reagents | Quantities (wts.) | |
|---|---|---|
| 545OH | 1 | eq. |
| SO₃•py | 5.37 | wts |
| DMF | 18.9 wts + 3 × 4.72 wts | |
| 10% NaOH | As req. | |
| EtOH | 4.00 | wts |

| | |
|---|---|
| IPA | 2.00 wts + 6 wts + 2 wts |

### Procedure

1. 545OH (1 wt.) and SO₃.Py (5.37 wts) in DMF (18.9 wts) was stirred under N₂ at 15-25° C. for 35-30 min then at 40-45° C. for 16-24 h.

2. The suspension was allowed to settle, and the supernatant was decanted and discarded.

3. The solid was slurried three times in DMF (3×4.72 wts), decanting the supernatant to waste.

4. Chilled water (2.0 wts.) was added and the mixture was slowly warmed until the product dissolved. The pH was adjusted to 8.0-10.0 using 10% w/w aq. NaOH, maintaining the temperature below 25° C.

5. EtOH (4.00 wts) was added at 15-25° C. and the suspension was aged overnight at 5° C.±5° C.

6. The suspension was filtered, and the filter cake was washed with WA (2wts).

7. The product was re-slurried in WA (6 wts) at 15-25° C. then filtered again. The filter cake was washed with WA (2 wts.)

8. The solid was dissolved in 1.39 w/w NaHCO₃ in H₂O (18.0 wts), filtered through a 0.22 μm SFCA filter and charged to a UF retentate tank.

9. Ultrafiltration/diafiltration was performed over a 2 kDa MWCO filter against 1.39% w/w NaHCO₃ in H₂O.

10. The retentate was concentrated to ca. 5 volumes and charged slowly to cooled WA (20 wts.).

11. The suspension was aged overnight, filtered over Grade 1 filter paper, and the filter cake was dried to a constant mass.

### Step 2: Precipitation of the Crude Compound 1 Product

The previously disclosed sulfation procedure for Compound 1 in WO2009049370A1 was unsuitable for scale-up, as it required evaporation of the DMF solvent to dryness to yield the crude product. However, during an initial scale-up of Compound 1 synthesis, the crude product was noted to precipitate as a finely divided solid, facilitating the slurry purification discussed above. This precipitation was generally more reliable when a certain minimum quantity of polyol 545OH was employed, for example 20 g or more. A further precipitation step was then carried out after basification, which removed impurities including residual pyridine. The use of precipitation rather than evaporation of high-boiling DMF solvent enabled efficient manufacture of Compound 1 on a large scale.

### Steps 2-3: Extractive Removal of Low-Molecular Weight Impurities During Workup

A major issue in the synthesis of Compound 1 is the removal of the undesired homologous oligosaccharide impurities, present in the original maltotetraose starting material. As purified maltotetraose G4 is expensive, the cost effective use of impure G4 syrups without chromatographic purification is a key improvement in the previously disclosed process to make Compound 1. Unable to remove ester-protected derivatives of G2 (Compound 2) and G3 (Compound 3) by crystallization or precipitation during the synthesis, the inventors of the present invention surprisingly discovered that the impurities could be carried through and removed after the final sulfation reaction.

The pyridinium salts of trisaccharide impurity Compound 3 and disaccharide impurity Compound 2 were surprisingly discovered to have an unexpectedly high solubility in the DMF reaction solvent. Thus, the sulfation conditions were developed to encourage precipitation of Compound 1 while keeping Compound 2 and Compound 3 in solution. This difference in solubility was particularly surprising when compared to previously reported sulfonation conditions for other oligosaccharides, in particular oligosaccharide derivatives not having a hydrophobic aglycon, which separated as a thick oil under the sulfation conditions. In contrast, the method presented herein can yield the initial crude Compound 1 as a filterable solid (Step 2), suitable for slurry washing. The previously reported conditions for the sulfation of Compound 1 did not give a filterable solid, but instead required evaporation of the DMF reaction solvent to dryness, which may be impractical on a manufacturing scale. However, in the present method a lower volume of DMF reaction solvent was used to encourage precipitation of Compound 1 while keeping the undesired lower homologues in solution. Further, a less aggressive temperature profile was used during the sulfation reaction to encourage slower precipitation of Compound 1 as a filterable solid rather than a thick oil. Previous sulfation reactions to prepare Compound 1 used 60° C. throughout the sulfation reaction, whereas the present method used a reaction temperature of 15-25° C. for 35-30 min followed by 40-45° C. for 16-24 h.

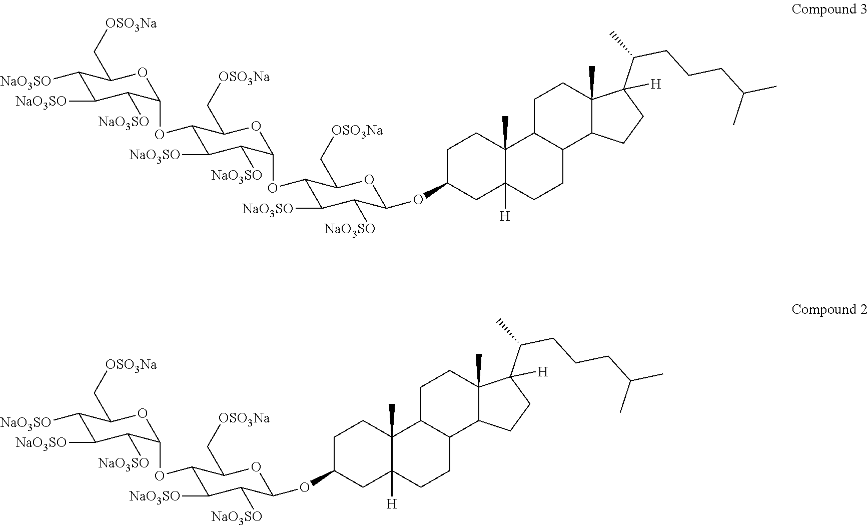

### Final Purification by Ultrafiltration

The use of dialysis/ultrafiltration for de-salting and final purification of Compound 1 has been disclosed previously [Yu, G. et al. Eur. J. Med. Chem. 2002, 37, 783-791; and Ferro, V. et al. J. Med. Chem. 2012, 55, 3804-3813]. This purification is considered extremely efficient, as it allows purging of all impurities that pass through the UF membrane (2000 Da MWCO).

Diafiltration against a solution containing a pharmaceutically acceptable cation is preferred to enable the product to be isolated in a pharmaceutically acceptable salt form of the product. In certain embodiments, the pharmaceutically acceptable cation is sodium, which can be formed, for example, by diafiltration of the product against a solution of sodium chloride in water.

The stability of Compound 1 requires that the compound is maintained at basic pH, or more conveniently at the pH of human blood (7.24). Optionally then, diafiltration against a pharmaceutically acceptable buffer solution at basic pH can yield a product with increased stability and shelf-life. A number of non-toxic buffers are available for this purpose, such as phosphate and citrate. The inventors of the present invention have used bicarbonate as it is volatile under HPLC analysis conditions when evaporative light scattering (ELS) detection is used. Performing the diafiltration/ultrafiltration against sodium bicarbonate (isotonic, 1.39%) in Water for Injection (Step 9 above) yields a product suitable for parenteral administration into humans, once it has been suitably sterilised. This was desirable due to the cost and limited availability of lyophilisation for bulk Compound 1 product, previously required for isolation of the final product as a solid.

Alternatively, if isolation of Compound 1 as a solid is required, the material may be precipitated by continuing on to Step 10 and Step 11 in the procedure above.

### Synthesis of Compound 4

The synthesis above can be adapted to give an improved convergent synthesis of Compound 4 as shown in Scheme 10. The use of the potentially hazardous azide building block used in WO2009049370A1 is avoided in favour of preparing the required propylstearamide aglycon by acylation of aminopropanol with stearoyl chloride. Glycosylation with G4BzSTol as for Compound 1 proceeds smoothly to yield glycoside 562Bz, which is then reacted on to form Compound 4 in a manner analogous to the synthesis of Compound 1 discussed above.

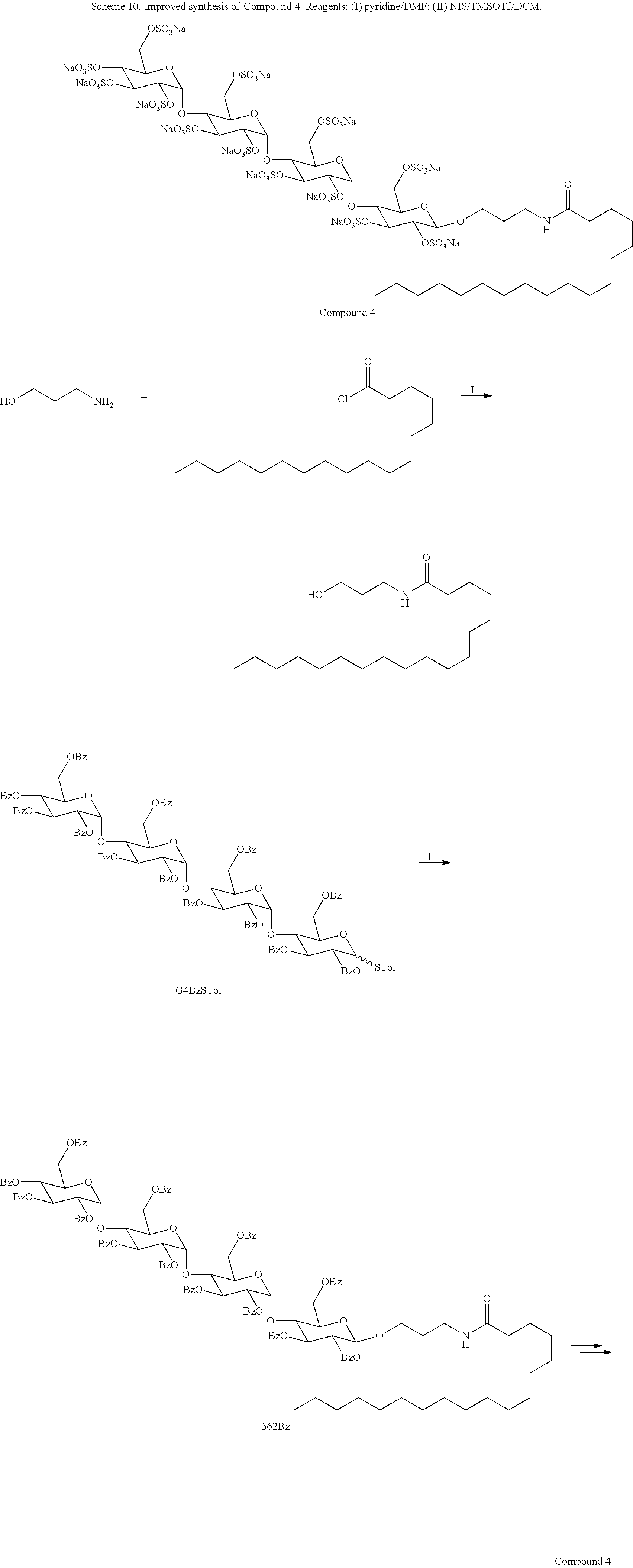

### Synthesis of Compound 5

### Overall Synthesis

The synthesis of Compound 5 was performed according to the four-stage synthesis outlined in Scheme 11. Each stage of the synthesis is discussed in further detail in the following sections. Importantly, the M5Ac starting material is depicted in Scheme 11 as a single pentasaccharide component for convenience, however it is actually a mixture of oligosaccharides from disaccharide to pentasaccharide, as shown in the structures below.

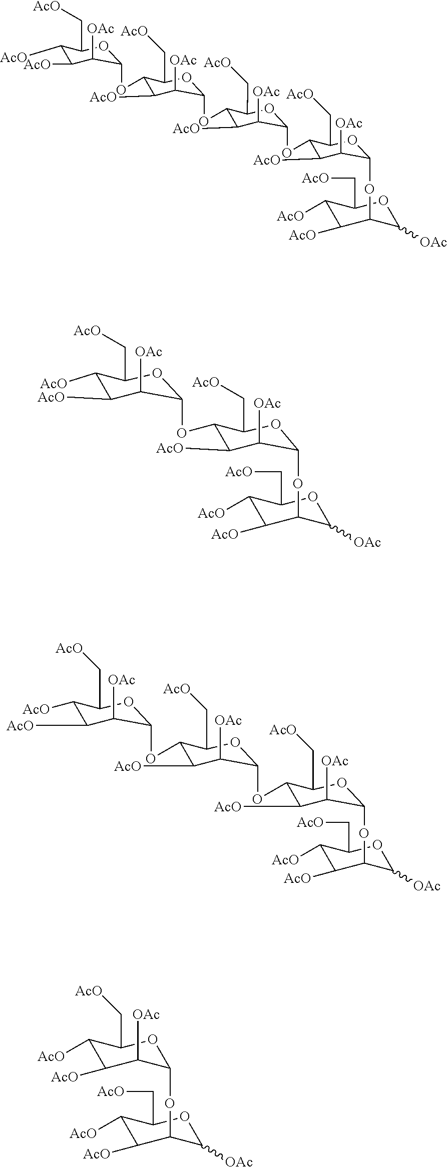

The ultimate source of the M5Ac starting material was the “neutral oligosaccharide fraction” isolated as a by-product of the manufacture of phosphomannan (Ferro, V., Fewings, K., Palermo, M. C., & Li, C. (2001). Large-scale preparation of the oligosaccharide phosphate fraction of Pichia holstii NRRL Y-2448 phosphomannan for use in the manufacture of PI-88. Carbohydrate Research, 332(2), 183-189. doi:10.1016/s0008-6215(01)00061-1). The neutral oligosaccharide fraction was shown therein to be comprised of a mixture of homologous mannooligosaccharides. The mixture was isolated by lyophilisation, and acetylated using acetic anhydride and pyridine as described previously (see WO2009049370A1).

The synthesis outlined in Scheme 11 enables enhancement in the amount of the pentasaccharide product (compound 5), which is the component with the higher molecular weight, as compared with the lower oligosaccharide homologues (i.e. di, tri and tetra-saccharide).

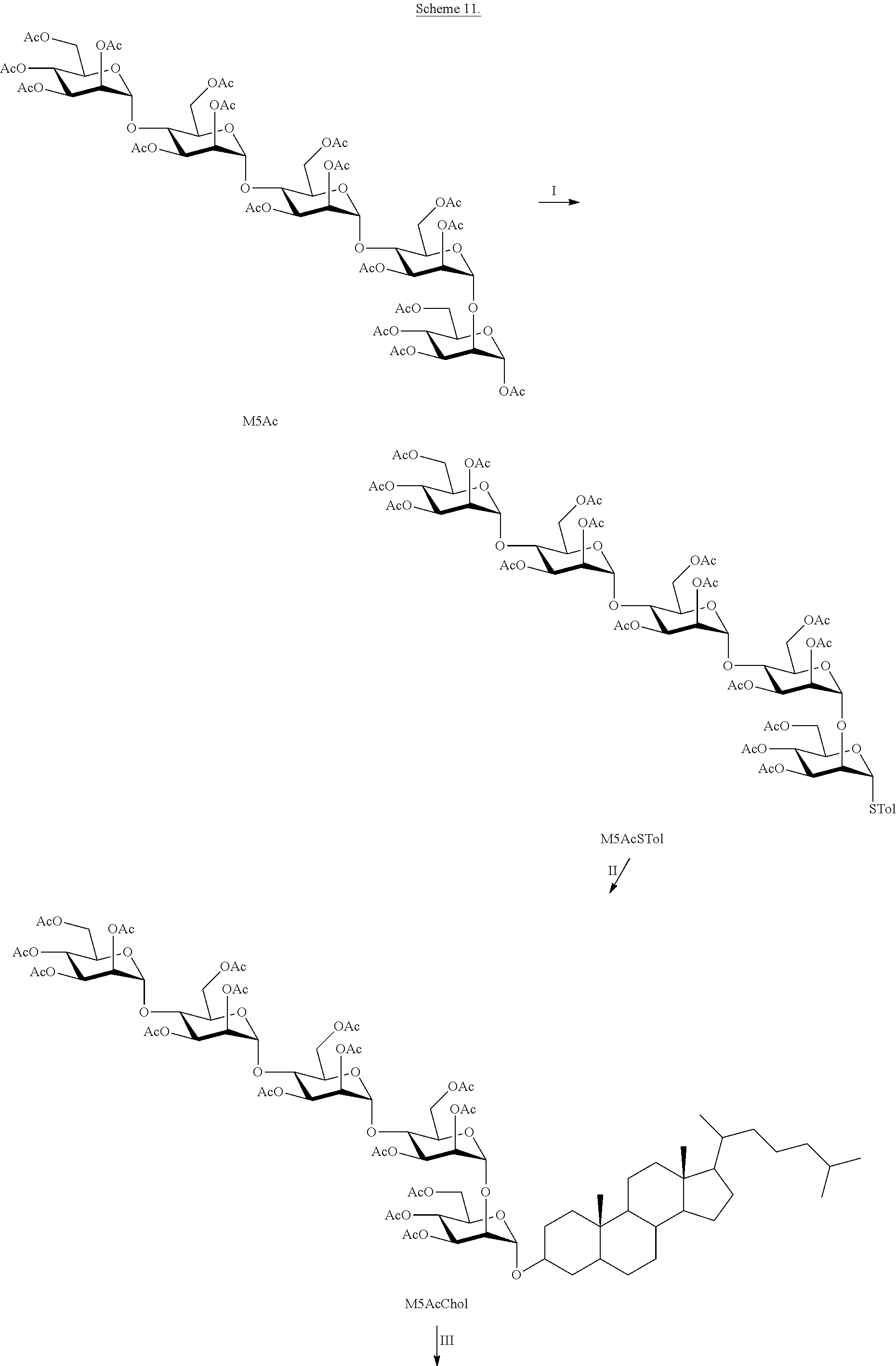

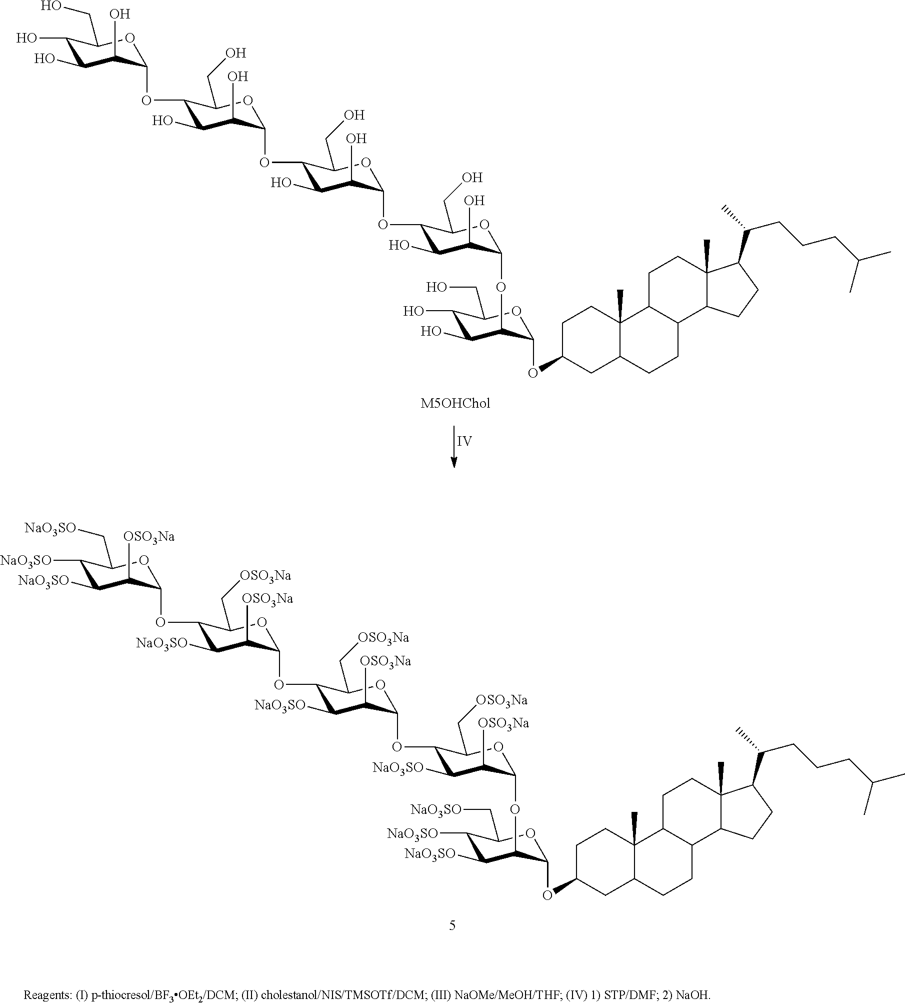

### Stage 1: Synthesis of M5AcSTol

The synthesis of M5AcSTo1 from M5Ac is outlined in Scheme 12 below. Briefly, the anomeric group of M5Ac is thiolated using para-thiocresol and BF₃·OEt₂ in dichloromethane.

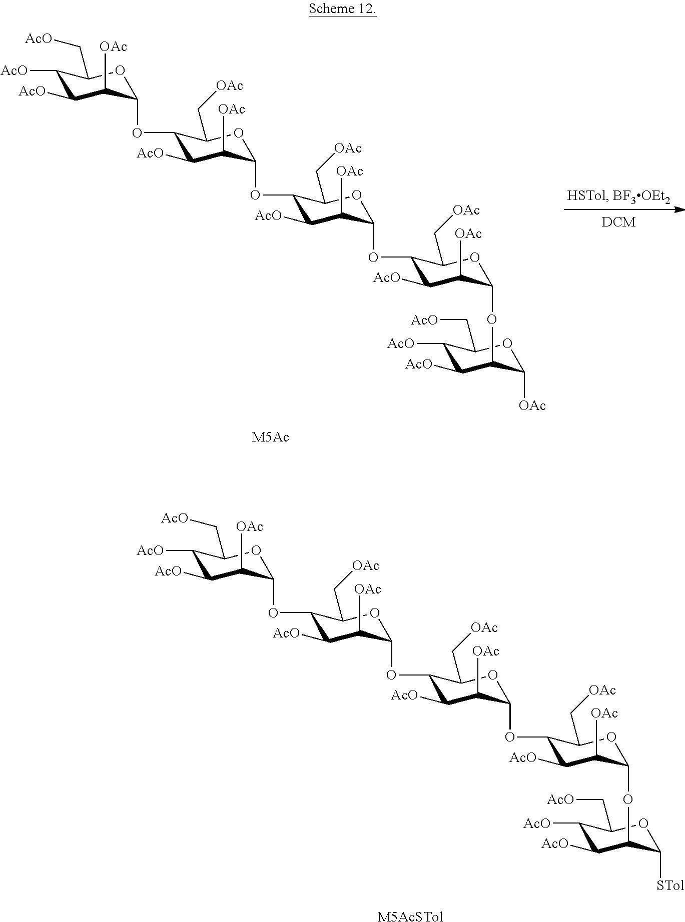

### Materials

| Reagents | Quantity (wts.) | | Quantity (g) | |
|---|---|---|---|---|
| M5Ac | 1 | wt. | 5 | g |
| Thiocresol | 1.54 | wt. | 0.62 | g |
| DCM | 1 × 2 wt., 1 × 0.2 wt. | | 10 mL, 1 mL | |
| BF₃•Et₂O | 1.8 | wt. | 0.72 | mL |
| Et₃N | 2.10 | wt. | 0.95 | mL |
| Ethyl acetate | 8 | wts. | 40 | mL |
| 10% aq. KOH/5% aq. Na₂S₂O₃ | 2 × 5.0 | wts. | 2 × 25 | mL |
| 10% aq. NaCl | 2 × 5.0 | wts. | 2 × 25 | mL |

### Procedure

1. M5Ac (1 wt.) and thiocresol (451 mg, 1.54 equiv.) were charged to a three necked round bottom flask and dissolved in DCM (10 mL, 2 vol.). The solution was then warmed to 34° C. using a metal heating block.

2. BF₃·OEt₂ (0.5 mL) was added dropwise, maintaining the reaction at <36° C. during the addition.

3. The reaction was stirred at 35° C. for 5 hours, then cooled to ambient temperature and aged for a further 11 hours. The reaction was further warmed to 36° C. and stirred for a further 2 h 45 mins.

4. BF₃·OEt₂ (0.28 mL) was added dropwise.

5. The reaction was stirred at 36° C. for a further 17 hours, when DCM (6 mL) was added.

6. Thiocresol (94.5 mg) was added, followed by the dropwise addition of BF₃.Et₂O (0.20 mL). The reaction was heated at 36° C. for a further 4 hours 30 minutes.

7. The reaction was quenched with the addition of Et₃N (1.2 mL) in EtOAc (40 mL).

8. The organic phase was successively washed with the following: 1×25 mL 10% aq. NaCl, 2×25 mL 10% aq. KOH/5% aq. Na₂S₂O₃, and 1×25 mL 10% aq. NaCl.

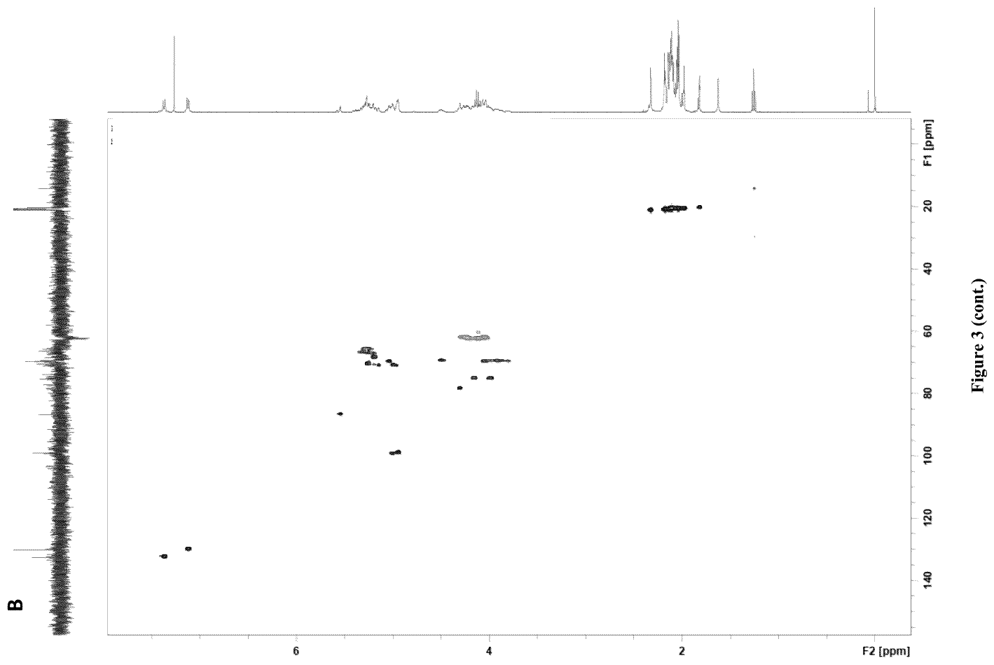

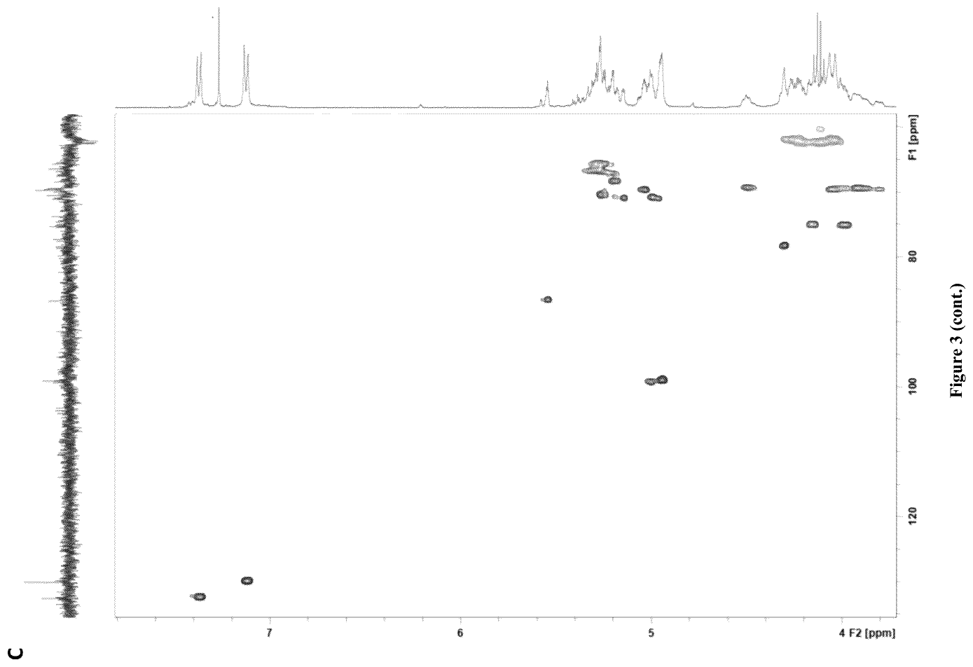

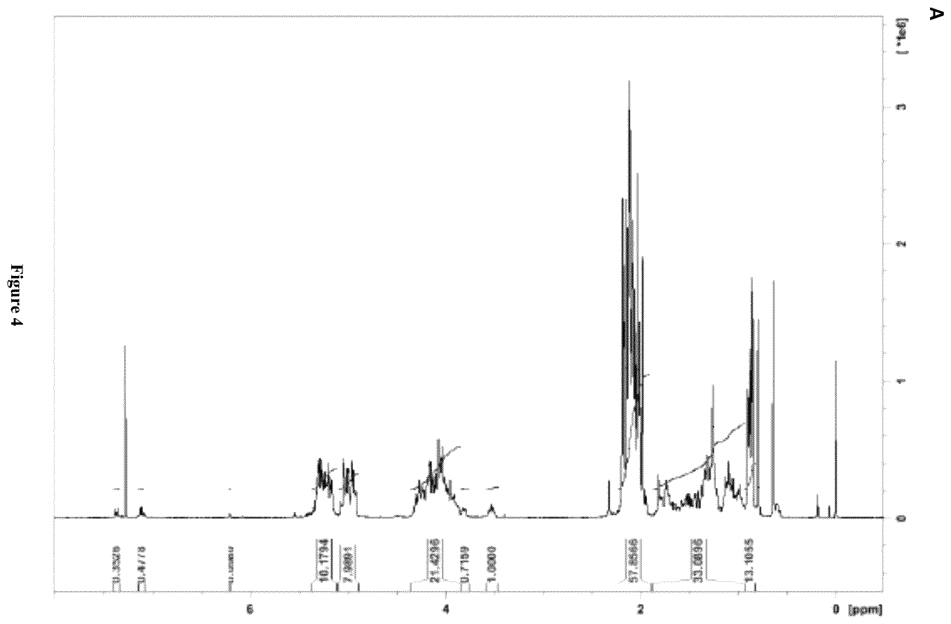

9. The aqueous phases were separated. The organic phase was concentrated under reduced pressure to give the product M5AcSTol as a beige foam (5.44 g). The ¹H NMR spectrum of the product is shown at FIG. 3A, and the HSQC NMR spectrum is shown at FIGS. 3B and 3C.

### Stage 2: Synthesis of M5AcChol

The synthesis of M5AcChol from M5AcSTol is outlined in Scheme 13 below. Briefly, cholestanol is glycosylated using M5AcSTol, NIS and TMSOTf in dichloromethane.

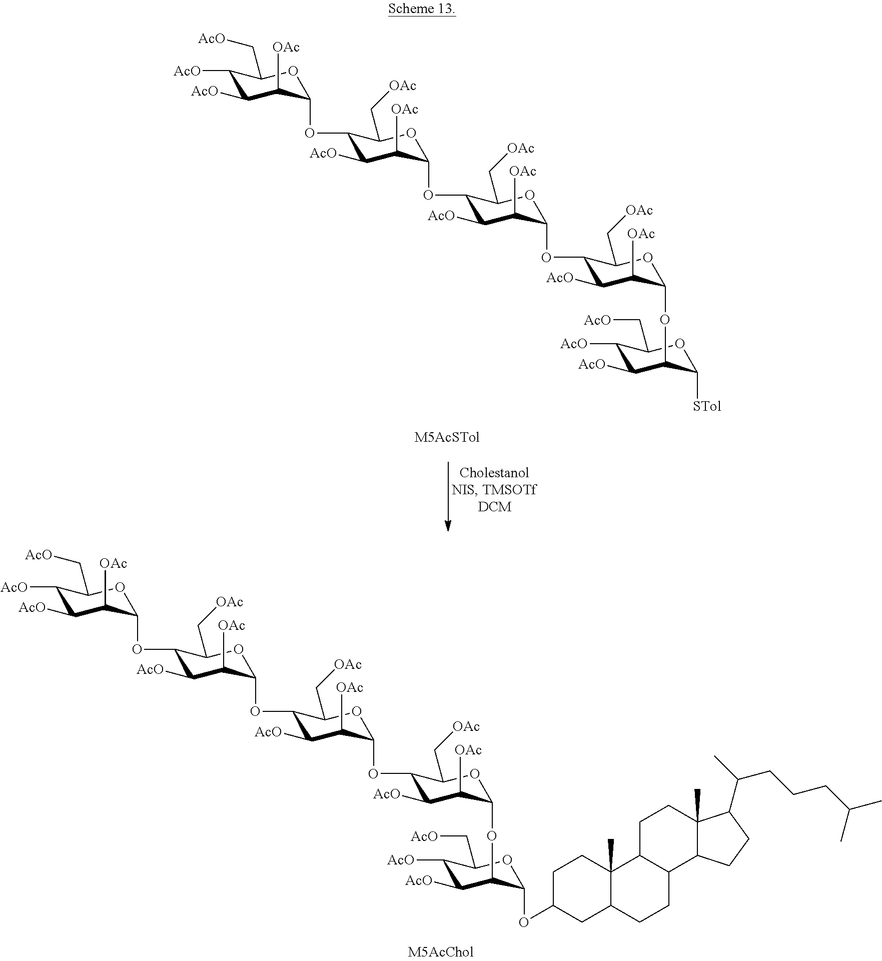

### Materials

| Reagents | Quantity (wts.) | | Quantity (g) | |
|---|---|---|---|---|
| M5AcSTol | 1 | wt. | 5.21 | g |
| 5α-cholestan-3β-ol | 1.47 | wt. | 1.85 | g |
| DCM | 8 wt. + 0.11 wt. | | 41.68 mL + 0.573 mL | |
| NIS | 2 | wt. | 1.458 | g |
| TMSOTf | 0.67 | wt. | 0.483 | mL |
| 10% aq. KOH/5% aq. | 2 × 5.0 | wts. | 2 × 26.05 | mL |
| Na₂S₂O₃ | | | | |
| 5% aq. NaHCO₃ | 1 × 5.0 | wts. | 1 × 26.05 | mL |
| 10% aq. NaCl | 1 × 5.0 | wts. | 1 × 26.05 | mL |
| EtOAc | 3 | wts. | 15.63 | mL |
| Heptane | 20 | wts. | 104.20 | mL |

### Procedure

1. A solution of M5AcSTo1 (1.0 wts) and 5α-cholestan-3β-ol (1.80 g, 1.47 equiv.) in DCM (40 mL) was stirred under N₂ for 30-60 min.

2. The mixture was cooled to 6° C. and NIS (1.40 g) was added to give a dark orange/red partial suspension. TMSOTf (0.48 mL) in DCM (0.5 mL) was added dropwise, and stirred for 20 mins.

3. The mixture was stirred at 5° C. overnight.

4. After 16 hours, the reaction was quenched by adding a solution of 10% aq. KOH/5% aq. Na₂S₂O₃ (5 wts.) at 0-5° C. during addition.

5. The phases were separated, and the organic phase was washed successively with the following: 1×25 mL 10% aq. KOH/5% aq. Na₂S₂O₃ solution, 1×25 mL 10% aq. NaHCO₃, and 1×25 mL 10% aq. NaCl.

6. The organic phase was concentrated under reduced pressure to give the crude material as a dark brown oil (7.87 g).

7. The crude material was dissolved in EtOAc (15 mL) and stored overnight at 5° C.

8. Heptane (100 mL) was cooled to 5° C. (ice/water bath) and the crude EtOAc solution was added dropwise to give a suspension which was aged at 5° C. for 1 hour.

9. The solid was isolated by filtration (Whatman #1 paper), the solids and mother liquors were analysed by TLC (visualised using 10% H₂SO₄ in IMS).

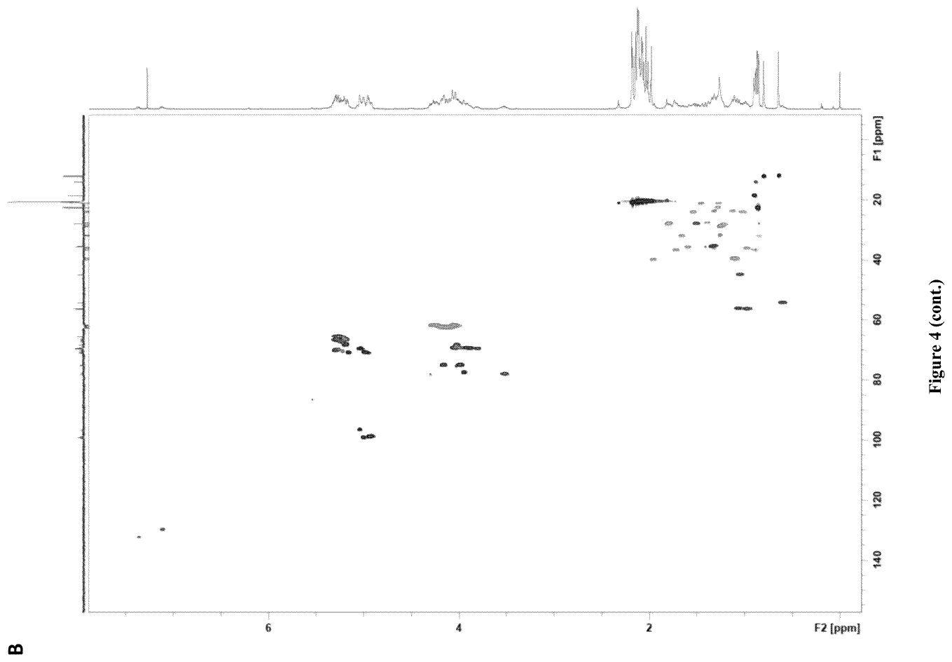

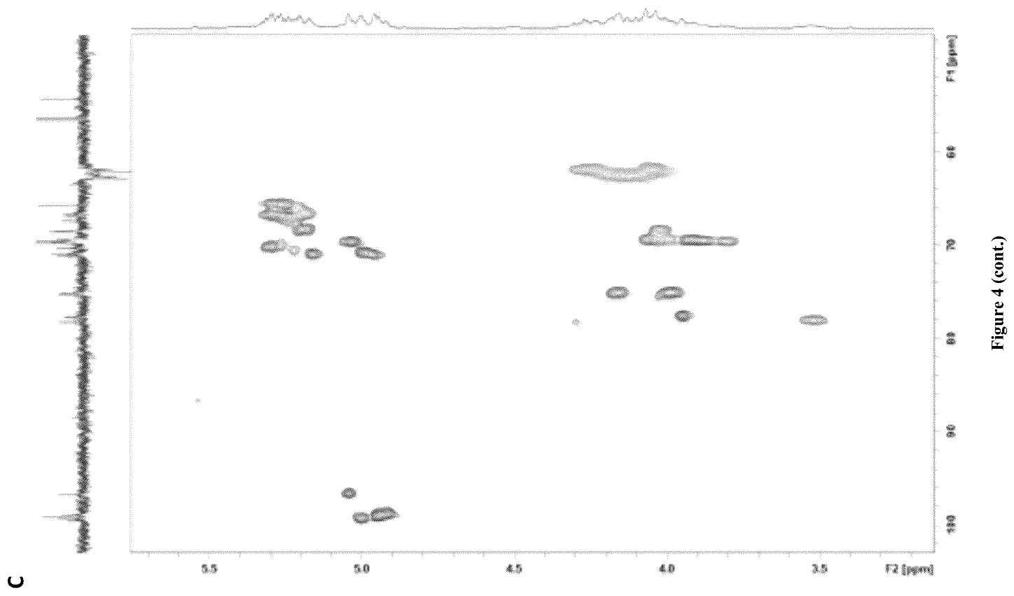

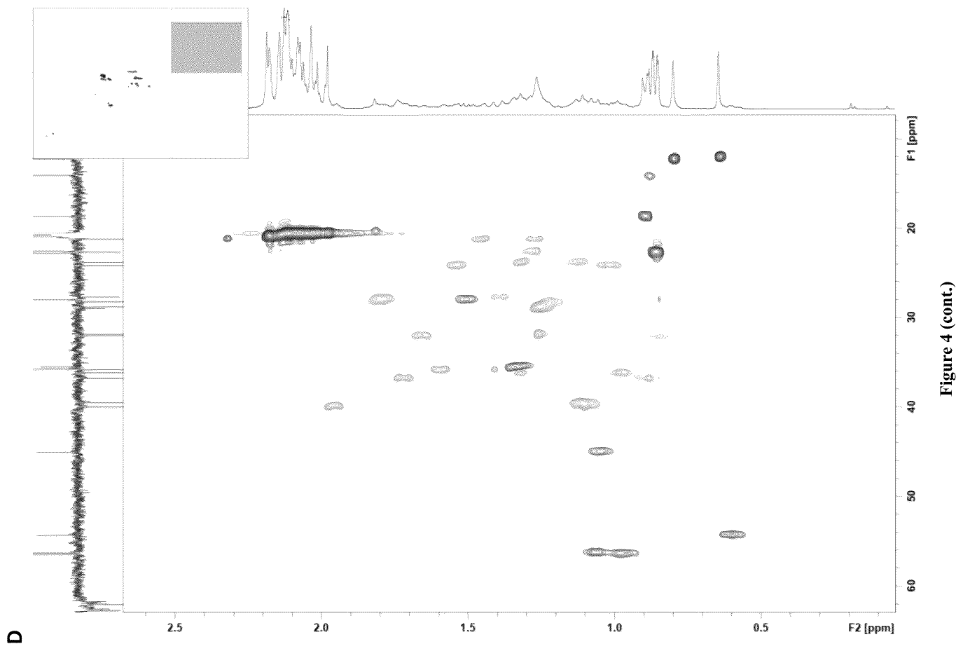

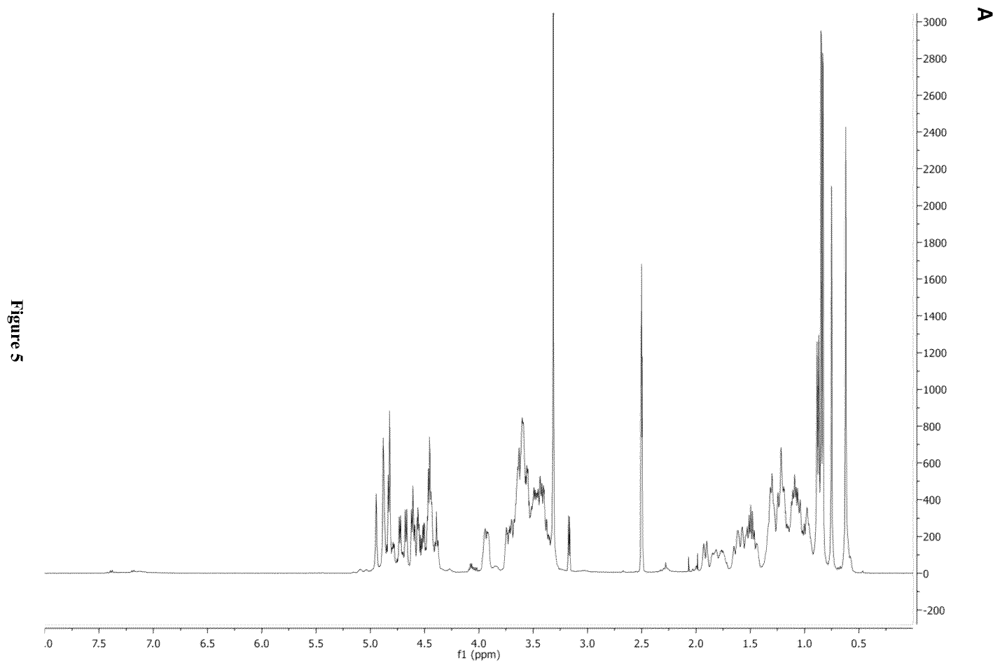

7. The solids were dried under reduced pressure to give a light tan powder M5AcChol product (4.66 g, 0.93 wts.) which was analysed by NMR, see FIG. 4 (solids ¹H (FIG. 4A) and solids HSQC (FIG. 4B-D)).

### Stage 3: Synthesis of M50HChol

Global deprotection of peracetate M5AcChol was accomplished using standard Zemplén transesterification with methanol, catalysed by methoxide as shown in Scheme 14.

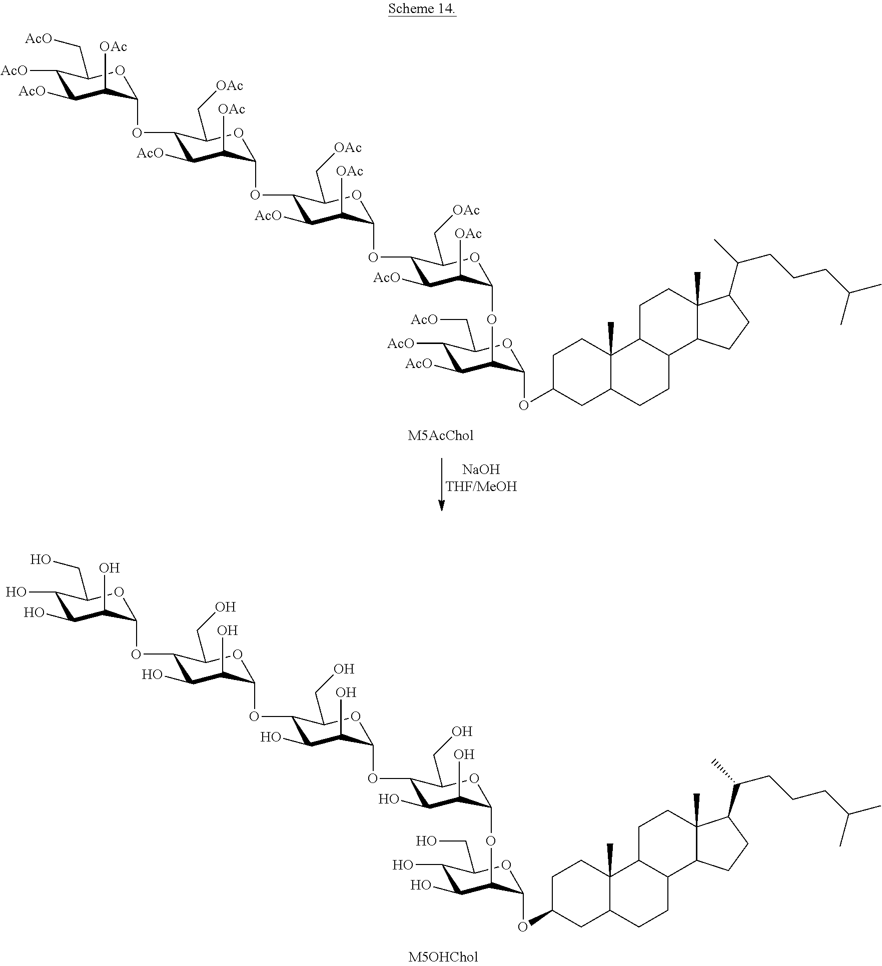

### Materials

| Reagents | Quantities (wts.) | | Quantities (g) | |
|---|---|---|---|---|
| M5AcChol | 1 | wt. | 4.62 | g |
| THF | 2 | vol. | 9.5 | mL |
| MeOH | 4 | vol. | 19 | mL |
| 25% NaOMe in MeOH | 3.45 | wt. | 2 | mL |

| AcOH | As req. | | |
|---|---|---|---|
| EtOAc | 10 vol. + 2 × 1 vol. | | 47 g + 2 × 4.7 g |
| H₂O | As req. | | |
| Acetonitrile | 59 | wt. | |

### Procedure

1. To a 100 mL 3-neck round bottom flask (fitted with N₂ bubbler, magnetic stirrer and thermometer) was charged M5AcChol (4.62 g, 1 equiv., 1 wts.), THE (9.5 mL, 2.0 vol.) and MeOH (19 mL, 4 vol.).

2. The dark brown solution was heated to 36° C. using a heating block.

3. 25% Sodium methoxide in methanol (2.0 mL) was added, monitoring the temperature.

4. The resultant suspension was warmed to 51° C. and stirred for 4 hours.

5. The reaction was allowed to cool to ambient temperature, and stirred overnight.

6. Glacial acetic acid (400 μL) was added (portion-wise) until the pH was neutral and a sample was analysed by TLC (visualised with 10% H₂SO₄ in IMS).

7. A sample of the reaction mixture was filtered, and the resulting solid and mother liquors were analysed by TLC (visualised with 10% H₂SO₄ in IMS).

8. The reaction mixture was concentrated to a solution in water under reduced pressure.

9. The crude product material was precipitated by the addition of MeOH and EtOAc and the supernatant was decanted.

10. The solids were dried under reduced pressure to give a tan solid (3.67 g, 122% yield, 0.79 wts.) containing crude M50HChol.

### Purification by Ultrafiltration (Diafiltration)

11. The crude solids were dissolved in water (50 mL) and passed through a 0.22 m SFCA filter rinsing with water (10 mL+5 mL) to give a dark brown solution.

12. The product solution underwent diafiltration against water for 7 diavolumes using a 30 kDa size exclusion membrane (vivaflow unit, 1 diavolume=65 mL).

13. After the diafiltration was completed, the retentate was concentrated to 20-30 mL.

14. The retentate was flushed from the unit and the membrane rinsed with water (5 mL+20 mL).

15. The combined retentate was concentrated under reduced pressure to 3.44 g, water (1.5 mL) was added to give a final retentate weight of ˜5 g.

16. The material was precipitated by adding the retentate dropwise to cold acetonitrile (120 mL) (ice/water bath).

17. The resulting suspension was aged for 1 hour, then filtered (cloth) under a blanket of inert gas.

18. The isolated solids were washed with EtOAc.

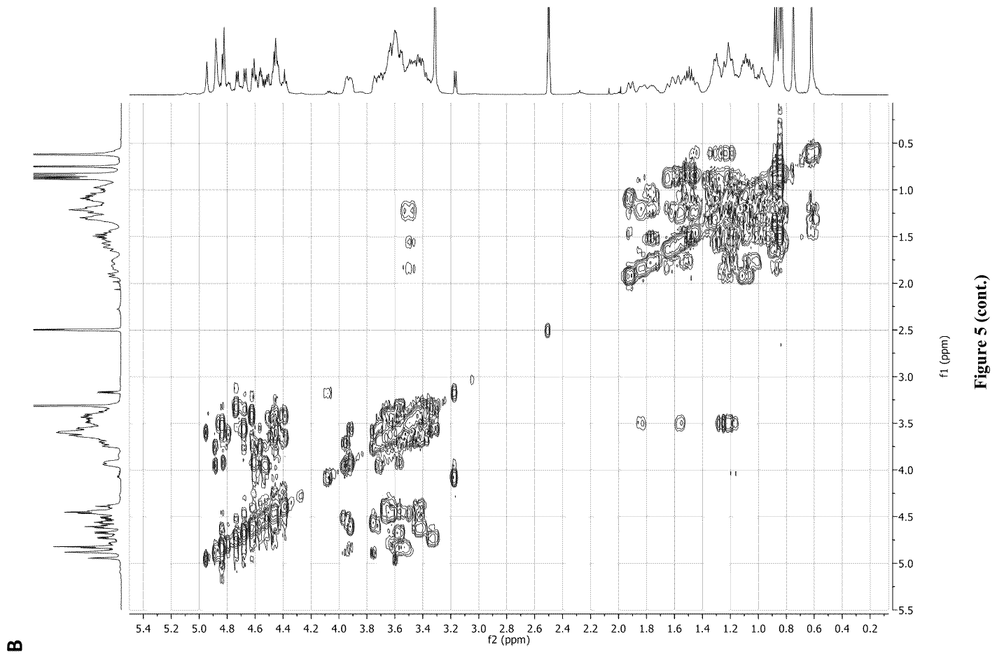

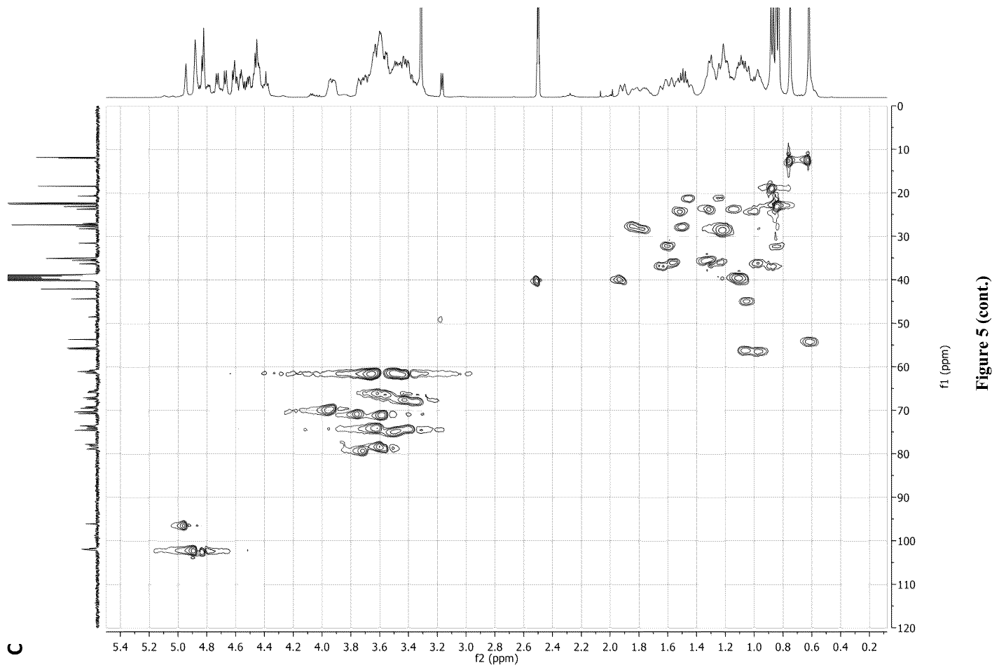

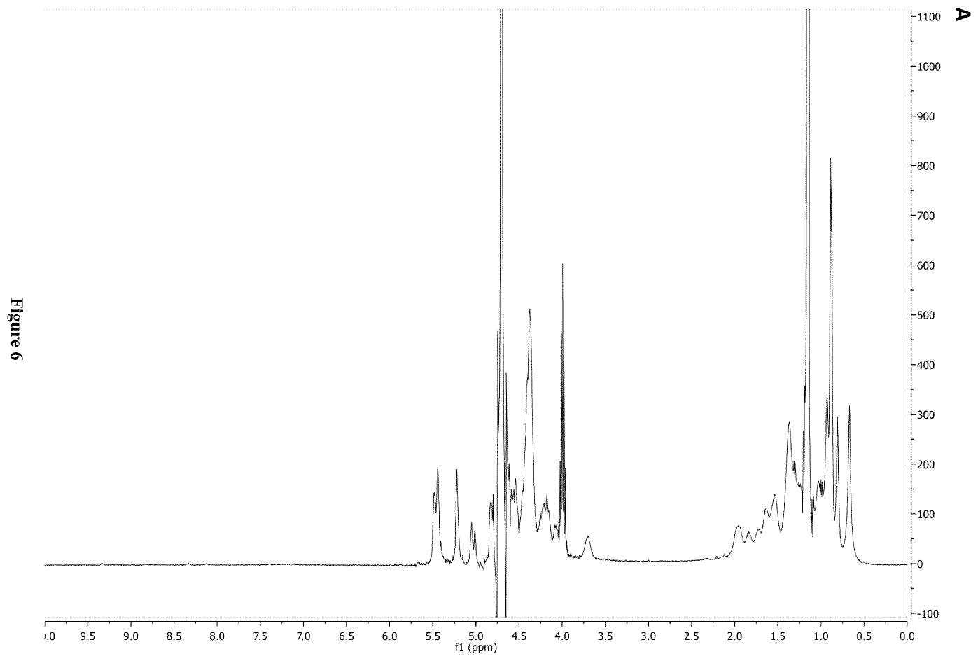

19. The polyol was dried under reduced pressure to yield M5OHChol as a tan solid (1.72 g, 0.58 wts.), which was analysed by NMR, see FIG. 5 (¹H (FIG. 5A), COSY (FIG. 5B) and HSQC (FIG. 5C)). The product composition was analysed by HPLC (area/area) and had the following components: disaccharides 2.8%, trisaccharides 14.8%, tetrasaccharides 46.3%, pentasaccharides (i.e. M50HChol) 28.1%, unidentified/other 8.1%.

### Stage 4: Synthesis of Compound 5

The synthesis of Compound 5 uses a combination of sulfur trioxide pyridine complex (˜3 eq. per hydroxyl group) in DMF solution as outlined in Scheme 15.

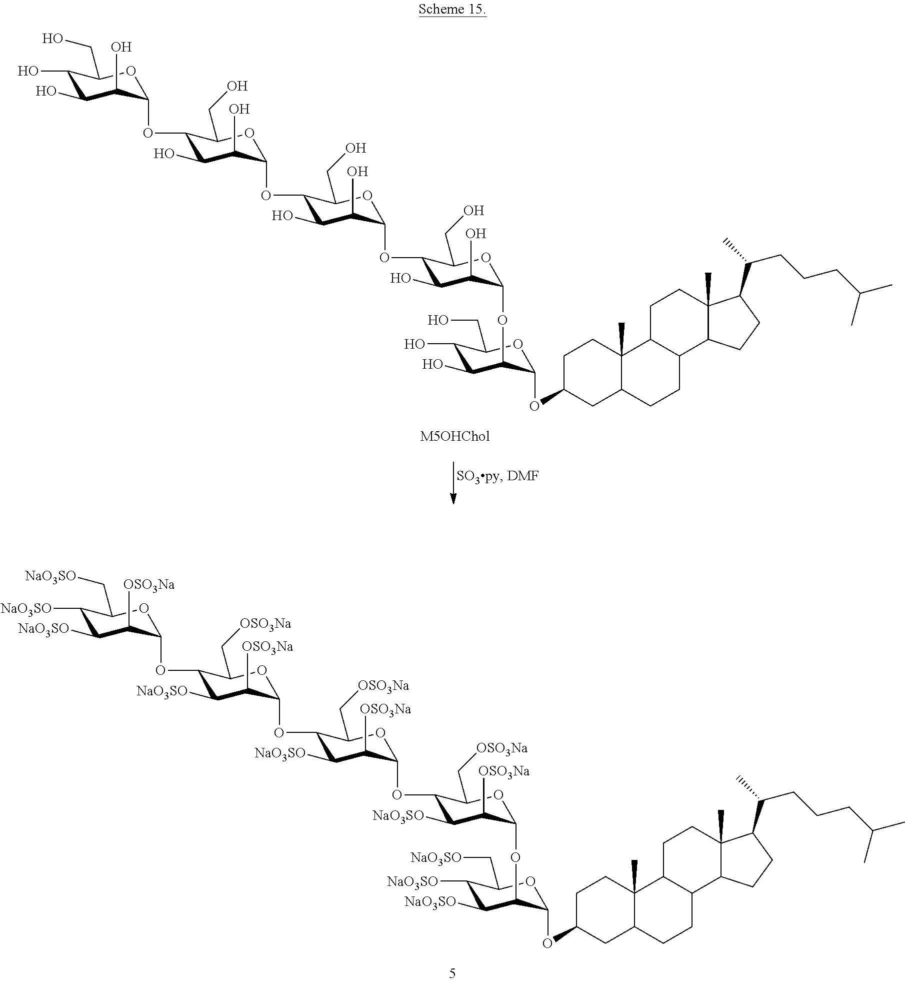

### Materials

| Reagents | Quantities (wts.) | |
|---|---|---|
| M5OHChol | 1 | eq. |
| SO₃•py | 39.10 | wts |
| DMF | 57.2 wts + 40.2 wts | |
| 10% NaOH | As required | |
| EtOH | 9 | vol. |
| IPA | 2.5 vol. + 16 vol. | |
| H₂O | 8 | vol. |
| NaCl | 2.84 | wt. |

### Procedure

1. To a 250 mL 3-neck round bottom flask (fitted with N₂ bubbler, large magnetic stirrer and thermometer) was charged M5OHChol (1.672 g, 1 equiv., 1 wts.) and DMF (95 mL, 57 vol.).

2. The resulting mixture was stirred until a brown hazy solution formed. The reaction mixture was cooled to 5° C. (±5° C.) by use of an ice bath.

3. SO₃-py (9.44 g, 39 equiv.) was added in one portion to the cold mixture.

4. The reaction was stirred for a further 30 min before the ice/water bath was removed. The reaction mixture was subsequently heated to 45° C.

5. The reaction was left to stir overnight at 45° C.

6. The reaction was allowed to cool to ambient temperature; the mixture still appeared as a hazy solution.

7. A sample of the reaction mixture was analysed by TLC to confirm full consumption of starting material.

8. To a ˜60 mL portion of the reaction mixture; EtOAc (18 mL) was added without stirring.

9. Stirring was started to facilitate full mixing of the layers and then stopped after (5-10 seconds) to allow the precipitated material to settle.

10. The solid was allowed to settle and the supernatant was removed by vacuum transfer through a dip tube with a sintered tip.

11. A solution of DMF (40 mL) and EtOAc (9 mL) was added, and the mixture stirred vigorously.

12. Once the material was completely suspended, the stirring was stropped and left to settle over 1-2 hrs.

13. The supernatant was removed by vacuum transfer.

14. The resultant slurry was dissolved in water (10 mL).

15. 10% NaOH solution (4 mL) was added (resulting in a pH of 6-7). Then 10% NaOH solution (1 mL) was added, (resulting in a pH of 14). Following this, 2M HCl solution (0.2 mL) was added, (resulting in a pH of 7). Finally, 10% NaOH solution (0.1 mL) was added, (resulting in a pH of pH 8-9).

16. The supernatant was removed by vacuum transfer.

17. The solids were redissolved in water (14 mL)

18. Ethanol (5.5 mL) was added dropwise to cause precipitation.

19. The solids were filtered using a Buchner filter and washed with IPA (4 mL).

Filtration was Slow Due to a Partial Blockage; However, this was Manageable on this Small Scale.

20. The solids were dried under reduced pressure.

21. The obtained dry solid was a fine powder containing larger clumps with a beige/light brown colour (1.95 g “72% yield”, 1.82 wts.).

### Diafiltration

22. The obtained solids (1.87 g) were dissolved in 1.39% aq. NaHCO₃ solution (100 mL)

23. The solution was filtered through a 0.2 μm SFCA filter. An additional 50 mL portion of 1.39% NaHCO₃ was used to rinse the flask and filter and to dilute the solution to the desired concentration (12.4 mg/mL).

24. The product solution underwent diafiltration against 1.39% aq. NaHCO₃ solution for 7 diavolumes with a 2 kDa size exclusion membrane (vivaflow unit, 1 diavolume=150 mL).

25. After 7 diavolumes, the retentate was concentrated to 20-30 mL

26. The retentate was flushed from the unit and the membrane rinsed with an additional 2×20 mL, 1.39% aq. NaHCO₃ solution.

27. The combined retentate and flushes (65 mL) were transferred to a stirred Amicon ultrafiltration cell fitted with a 1 kDa size exclusion membrane to further concentrate the material to 10-15 mL total volume.

28. The material was precipitated by adding the concentrated mixture dropwise into cold IPA (80 mL) (ice/water bath).

29. The solids were removed by filtration washing with IPA (20 mL). The collected solids were dried under reduced pressure.

30. The obtained dry solid was a fine powder containing larger clumps with a beige/light brown colour (1.64g “61% yield”, 1.72 wts.)

### Precipitation

31. EtOAc was added in 0.1 mL portions up to a total of 0.6 mL. Further additions of a few drops showed minimal amounts of further precipitation. The thick flocculant suspension settled into a paste like solid.

32. The supernatant was removed by syringe and the remaining solids were washed with DMF (5 mL)/EtOAc (0.6 mL)

33. The wash resulted in a finely dispersed suspension which slowly settled to a paste like solid.

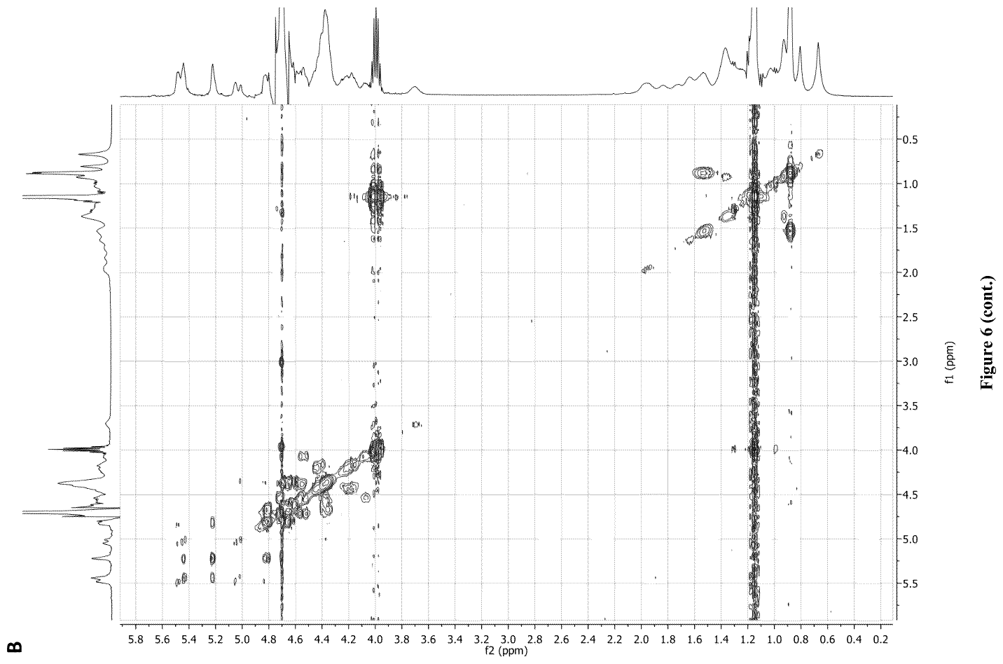

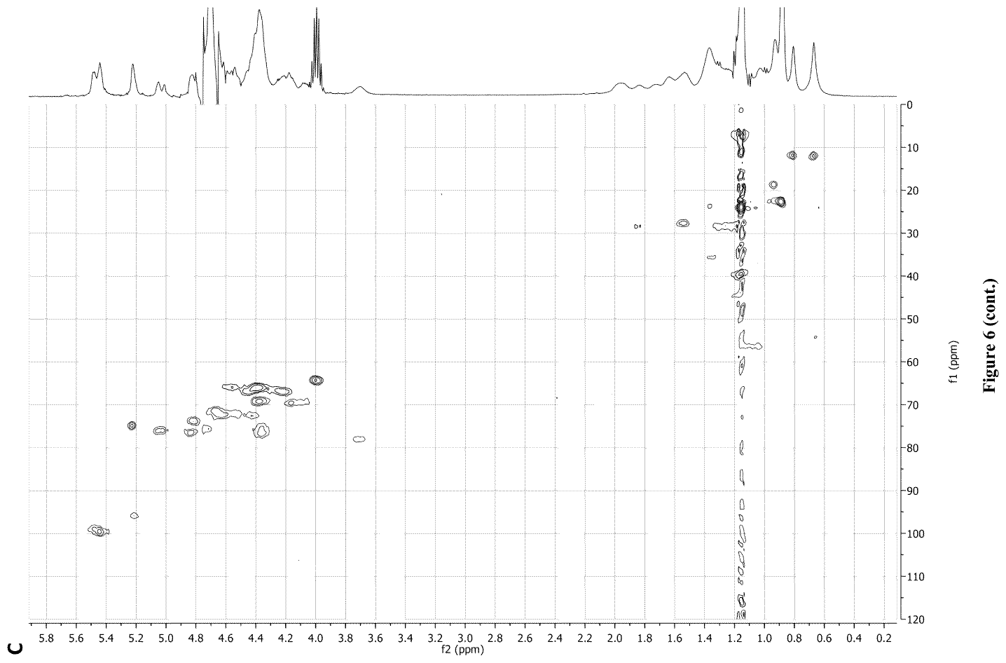

34. The supernatant was removed by syringe, yielding Compound 5 as a mixture with other mannooligosaccharide homologues, which was analysed by NMR, see FIG. 6 (¹H (FIG. 6A), COSY (FIG. 6B) and HSQC (FIG. 6C)). The product composition was analysed by HPLC (area/area) and had the following components: sodium 53.8%, disaccharides 0.3%, trisaccharides 5.5%, tetrasaccharides 20.6%, pentasaccharides (Compound 5) 19.8%.

HPLC comparison of the sulfated product with the unsulfated polyol precursor

M5OHChol showed that levels of Compound 5 had been enriched by partial removal of the lower homologues according to the inventive method. Table 1 shows the relative abundances of oligosaccharide homologues from di- to pentasaccharide before and after sulfation. The relative abundance of the pentasaccharide Compound 5 increased from 30.9% to 42.8% during the sulfation process, while the relative abundances of the lower homologues (di- to tetrasaccharides) all decreased to varying degrees.

TABLE 1
Relative abundance of mannooligosaccharides before and after sulfation.
Absolute abundances (HPLC area/area) reported above have been
normalised to total 100% for the selected chain lengths.

| | Relative Composition of | Relative Composition of |
|---|---|---|
| Homologue | Polyol | Sulfated Mixture |
| Disaccharide | 3.0 | 0.7 |
| Trisaccharide | 16.0 | 11.8 |
| Tetrasaccharide | 50.1 | 44.7 |
| Pentasaccharide | 30.9 | 42.8 |
| Total | 100.0 | 100.0 |

Although the procedure for Compound 5 was not as efficient as that illustrated for Compound 1 above, the skilled person will appreciate that the absolute abundance of the required pentasaccharide fraction of mixture M5OHChol was only 28% before sulfation (area/area by HPLC), whereas the process illustrated above for Compound 1 began with G4 syrup containing at least 50% of the required tetrasaccharide (area/area by HPLC on dried basis). In addition, while the preparation of Compound 1 was extensively optimised for GMP manufacture, the preparation of Compound 5 was carried out with minimal optimisation on a laboratory scale, and in view of this, the purity achieved for Compound 5 was not as high as that reported for Compound 1. However, a person of skill in the art would realise that the preparation of Compound 5 could be further optimised according to the methods illustrated above. Without being limited, further improvements to the preparation of Compound 5 might result from changing some of the factors and conditions described above. These may include but should not be limited to:
beginning with M5Ac starting material containing a higher proportion of pentasaccharide;
optimising the quantities of sulfur trioxide pyridine complex and polar aprotic solvent (e.g. DMF) used in the sulfation reaction based on the relative solubilities of the different homologues; and/or
optimising the composition of the wash steps using a dipolar aprotic solvent.

The skilled person will appreciate that the examples disclosed herewith with respect to the synthesis of Compound 1, Compound 4, and Compound 5 could be adapted without undue burden to the synthesis of other sulfated oligosaccharides having a hydrophobic aglycon, in particular those having a maltotetraose or manno-oligosaccharide backbone.

Further illustration of the utility of the inventive methods can be gained from the comparison of these methods with similar methods from the literature. For example, the preparation of PI-88 on a scale suitable for clinical trial use has been reported previously. The phosphomannan starting material for the preparation of PI-88 is based on the same mannooligosaccharide backbone used herein for the preparation of Compound 5 (indeed, the two oligosaccharide mixtures are ultimately derived from the same biological source as mentioned above). However, there are important differences in that the M5Ac oligosaccharides used here do not contain phosphate groups, but instead feature a hydrophobic aglycon. It follows that the method reported here for sulfation of Compound 5 must necessarily have some similarities to the literature methods for the production of PI-88, as they are chemically related products. However, the literature methods for PI-88 generally do not result in the useful enrichment of the higher oligosaccharides, and the purging of the lower homologues, in contrast with the inventive method.

It will be appreciated that, although specific embodiments of the invention have been described herein for the purpose of illustration, various modifications may be made without deviating from the spirit and scope of the invention as defined in the following claims.

1. A method of producing a compound of Formula (I),
[X]ₙY−ZR¹R² Formula (I)
wherein:
X and Y are any D- or L-hexose or pentose, wherein each hydroxyl group not involved in a glycosidic linkage is substituted by a group W, and Y has an anomeric carbon atom;
W is SO₃M, and M is any pharmaceutically acceptable cation;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
the method comprising:
(A1) preparing a mixture comprising a compound of Formula (II), a reaction liquid, and a sulfur trioxide complex,
[X]ₙY−ZR¹R² Formula (II)
wherein:
n, Z, R¹ and R² are as defined in the compound of Formula (I), and
X and Y are any D- or L-hexose or pentose, wherein each hydroxyl group not involved in a glycosidic linkage is substituted by a group W, Y has an anomeric carbon atom, and W is H;
(A2) separating a solid from the mixture; and
(A3) washing the solid with a dipolar aprotic wash solvent to produce the compound of Formula (I).

2. The method of claim 1, wherein the dipolar aprotic wash solvent comprises dimethylformamide.

3. A method of producing a compound of Formula (II),
[X]ₙY−ZR¹R² Formula (II)
wherein:
X and Y are any D- or L-hexose or pentose, wherein each hydroxyl group not involved in a glycosidic linkage is substituted by a group W, Y has an anomeric carbon atom, and W is H;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
the method comprising:
(B1) mixing a compound of Formula (III) and a deprotecting agent to form the compound of Formula (II),
[X]ₙY−ZR¹R² Formula (III)
wherein:
n, Z, R¹ and R² are as defined in the compound of Formula (II), and
X and Y are any D- or L-hexose or pentose, wherein each hydroxyl group not involved in a glycosidic linkage is substituted by a group W, Y has an anomeric carbon atom, and W is a protecting group;
(B2) performing a membrane filtration to separate the compound of Formula (II) from one or more impurity;
wherein the membrane filtration uses a membrane with a pore size which is at least twice the molecular weight of the compound of Formula (II).

4. The method of claim 3, wherein the membrane filtration uses a membrane with a pore size which is at least five times the molecular weight of the compound of Formula (II).

5. A method of producing a compound of Formula (III),
[X]ₙY−ZR¹R² Formula (III)
wherein:
X and Y are any D- or L-hexose or pentose, wherein each hydroxyl group not involved in a glycosidic linkage is substituted by a group W, Y has an anomeric carbon atom, and W is a protecting group;
n is an integer from 2 to 6;
Z is O, and is linked to the anomeric carbon atom of Y;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
the method comprising:
(C₁) reacting a compound of Formula (IV), with a compound of Formula (V) to form a compound of Formula (III);
[X]ₙY−R³ Formula (IV)
wherein:
X, Y and n are as defined in the compound of Formula (III), and
R³ is an optionally substituted thioaryl group, and is linked to the anomeric carbon atom of Y;
HOR¹R²Formula (V)
wherein:
R¹ and R² are as defined in the compound of Formula (III).

6. The method of any one of claims 3 to 5, wherein the protecting group is an acetate or benzoate group.

7. The method of any one of claims 1 to 6, wherein R¹ is a bond, and R² is selected from the group consisting of optionally substituted steroidyl, optionally substituted C₁-C₁₀ alkyl-NHCO—C₁-C₂₆ alkyl, and optionally substituted C₁-C₁₀ alkyl-CONH—C₁-C₂₆ alkyl; wherein said optional substituents are selected from the group consisting of C₁-C₁₂ alkyl, C₂-C₁₂ alkenyl, C₂-C₁₂ alkynyl, and halogen.

8. The method of any one of claims 1 to 7, wherein R¹ is a bond, and R² is cholestanyl or propyl stearamide.

9. A method of producing a compound of Formula (IV),
[X]ₙY−R³ Formula (IV)
wherein:
X and Y are any D- or L-hexose or pentose, wherein each hydroxyl group not involved in a glycosidic linkage is substituted by a group W, Y has an anomeric carbon atom, and W is a protecting group;
n is an integer from 2 to 6;
Z is O, and is linked to the anomeric carbon atom of Y; and
R³ is an optionally substituted thioaryl group, and is linked to the anomeric carbon atom of Y;
the method comprising:
(D1) reacting a compound of Formula (VI), with a thioaryl compound to form a compound of Formula (IV);
[X]ₙY− V Formula (VI)
wherein:
X, Y and n are as defined in the compound of Formula (IV), and
V is W, and is linked to the anomeric carbon atom of Y.

10. The method of claim 9, wherein R³ is thiotolyl.

11. The method of any one of claims 1 to 10, wherein X and Y are glucose monosaccharide units.

12. The method of any one of claims 1 to 11, wherein X and Y are glucose monosaccharide units linked together with α-1,4 glycosidic linkages.

13. The method of any one of claims 1 to 10, wherein X and Y are mannose monosaccharide units.

14. The method of any one of claims 1 to 13, wherein n is 3 or 4.

15. A method of producing a compound of Formula (VII), 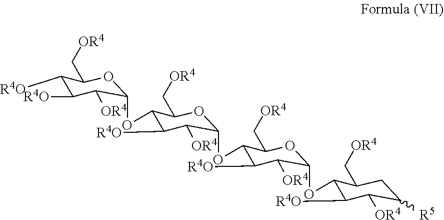
R⁴ is SO₃M, and M is any pharmaceutically acceptable cation;
R⁵ is OR¹R²;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond;
the method comprising:
(E1) providing a starting material comprising a mixture of maltooligosaccharides, said starting material comprising from 50% w/w to 95% w/w of maltotetraose on a dry weight basis;
(E2) reacting the starting material with an acyl halide, acyl anhydride, aroyl halide, or aroyl anhydride to form a compound of Formula (VIII), 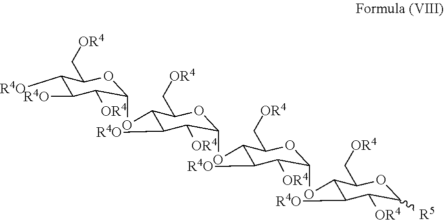
wherein:
R⁴ is an acyl or aroyl group, and
R⁵ is O-acyl or O-aroyl;
(E3) converting the compound of Formula (VIII) to a glycosyl donor of Formula (IX), 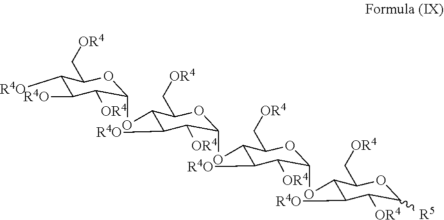
wherein:
R⁴ is as defined in the compound of Formula (VIII), and
R⁵ is a leaving group;
(E4) glycosylating a compound of Formula (X) with the glycosyl donor of Formula (IX) to form a compound of Formula (XI),
HOR¹R² Formula (X)
wherein R¹ and R² are as defined in the compound of Formula (VII), 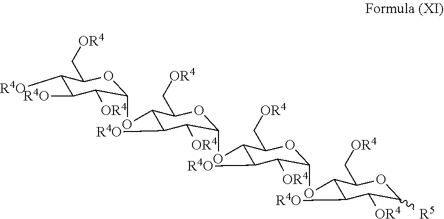
wherein:
R⁴ is as defined in the compound of Formula (VIII), and
R⁵ is as defined in the compound of Formula (VII);
(E5) removing acyl or aroyl protecting groups from the compound of Formula (XI) to form a first mixture comprising a compound of Formula (XII); 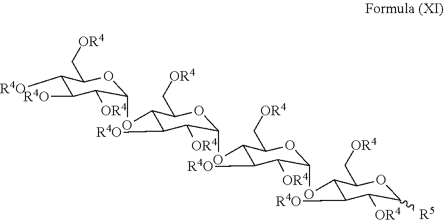
wherein:
R⁴ is OH;
R⁵ is as defined in the compound of Formula (VII);
(E6) subjecting the first mixture to membrane filtration to form a first purified composition comprising a compound of Formula (XII);
(E7) reacting the first purified composition with a sulfur trioxide complex in a reaction liquid to form a second mixture comprising a compound of Formula (VII); and
(E8) washing the second mixture with a dipolar aprotic wash solvent to form a second purified composition comprising a compound of Formula (VII).

16. The method of claim 15, which does not include a chromatography step.

17. The method of claims 15 or 16, wherein R¹ is a bond, and R² is selected from the group consisting of optionally substituted steroidyl, optionally substituted C₁-C₁₀ alkyl-NHCO—C₁-C₂₆ alkyl, and optionally substituted C₁-C₁₀ alkyl-CONH—C₁-C₂₆ alkyl; wherein said optional substituents are selected from the group consisting of C₁-C₁₂ alkyl, C₂-C₁₂ alkenyl, C₂-C₁₂ alkynyl, and halogen.

18. The method of any one of claims 15 to 17, wherein R¹ is a bond, and R² is cholestanyl or propyl stearamide.

19. A compound of Formula (VII) produced according to the method of any one of claims 15 to 17.

20. A compound of Formula (I) produced according to the method of claim 1 or 2.

21. A compound of Formula (XIII), or a salt thereof 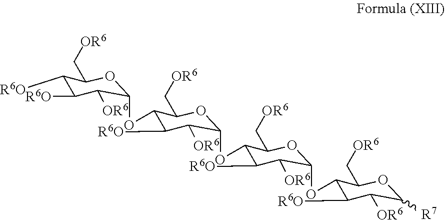
wherein:
R⁶ is an acyl or aroyl group; and
R⁷ is an optionally substituted thioaryl group.

22. The compound or salt thereof of claim 20, wherein R⁶ is benzoyl, and R⁷ is thiotolyl.

23. Use of the compound or salt thereof of claim 21 or 22 in the manufacture of a compound of Formula (VII); 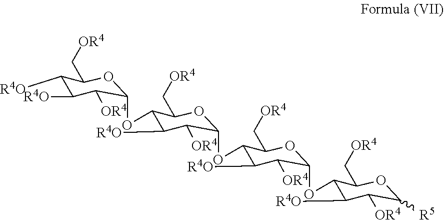
wherein:
R⁴ is SO₃M, and M is any pharmaceutically acceptable cation;
R⁵ is OR¹R²;
R¹ is C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹⁰—CONH—R¹¹, R¹⁰—NHCO—R¹¹, R¹⁰—CSNH—R¹¹, R¹⁰—NHCS—R¹¹, R¹⁰—CO—R¹¹, or is a bond;
R² is selected from the group consisting of optionally substituted terpenoidyl, optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, R⁸—CONH—R⁹, R⁸—NHCO—R⁹, R⁸—CSNH—R⁹, R⁸—NHCS—R⁹, R⁸—CO—R⁹, optionally substituted aryl, optionally substituted C₁-C₃₆ heteroalkyl, optionally substituted C₂-C₃₆ heteroalkenyl, optionally substituted C₂-C₃₆ heteroalkynyl, and optionally substituted heteroaryl;
R⁸ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and a bond;
R⁹ is selected from the group consisting of optionally substituted C₁-C₃₆ alkyl, optionally substituted C₂-C₃₆ alkenyl, optionally substituted C₂-C₃₆ alkynyl, optionally substituted C₄-C₃₆ cycloalkyl, optionally substituted C₄-C₃₆ cycloalkenyl, optionally substituted C₄-C₃₆ cycloalkynyl, optionally substituted aryl, optionally substituted C₁-C₃₆ heteroalkyl, optionally substituted C₂-C₃₆ heteroalkenyl, optionally substituted C₂-C₃₆ heteroalkynyl, optionally substituted heteroaryl, and hydrogen;
R¹⁰ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
R¹¹ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond.

24. A compound of Formula (XIV), or a salt thereof 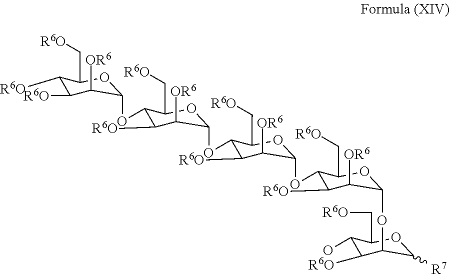
wherein:
R⁶ is an acyl or aroyl group; and
R⁷ is an optionally substituted thioaryl group.

25. The compound or salt thereof of claim 24, wherein R⁶ is acetyl, and R⁷ is thiotolyl.
